(19) United States
(12) Patent Application Publication
(10) Pub. No.: US 2010/0303776 A1
(43) Pub. Date: Dec. 2, 2010

(54) Methods and compositions for regulated expression of multiple nucleic acids

(75) Inventors: Richard Jude Samulski, Chapel Hill, NC (US); Kyson Xiaohuai Chou, Irvine, CA (US)

Correspondence Address: MYERS BIGEL SIBLEY & SAJOVEC, PO BOX 37428, RALEIGH, NC, 27627, US

(73) Assignee: The University of North Carolina at Chapel Hill

(21) Appl. No.: 12/799,095

(22) Filed: Apr. 16, 2010

Related U.S. Application Data
(60) Provisional application No. 61/170,024, filed on Apr. 16, 2009.

(51) Int. Cl. A61K 35/12; C12N 5/10; C07H 21/02; C12N 15/63; A61K 31/7105; C12P 19/34; A61P 43/00

(52) U.S. Cl. 424/93.21; 435/325; 536/23.1; 435/320.1; 514/44 R; 435/91.3

(57) ABSTRACT

The present invention provides a cell comprising a first heterologous nucleic acid construct and a second heterologous nucleic acid construct, wherein each of said first and second heterologous nucleic acid constructs comprises: A. a first nucleotide sequence encoding a nucleotide sequence of interest (NOI); and B. a second intronic nucleotide sequence comprising: i) a first set of splice elements defining a first intron that is removed by splicing to produce a first RNA molecule encoded by said first nucleotide sequence in the absence of activity at a second set of splice elements; and ii) the second set of splice elements defining a second intron different from said first intron, wherein said second intron is removed by splicing to produce no RNA molecule and/or to produce a second RNA molecule that is not encoded by said first nucleotide sequence, wherein each said first nucleotide sequence of each of said first and second heterologous nucleic acid constructs is different from one another and wherein each said second intronic nucleotide sequence of each of said first and second heterologous nucleic acid constructs is different from one another.

### STATEMENT OF PRIORITY

This application claims the benefit, under 35 U.S.C. §119(e), of U.S. Provisional Application No. 61/170,024, filed Apr. 16, 2009, the entire contents of which is incorporated by reference herein in its entirety.

### STATEMENT OF GOVERNMENT SUPPORT

Aspects of this invention were funded under Grant Nos. R21 76191 and P30 DK34987 of the National Institutes of Health. The U.S. Government has certain, rights in this invention.

### INCORPORATION OF SEQUENCE LISTING ON COMPACT DISK

The entire contents of the compact disk filed in identical duplicate and containing one file entitled “5470-495_ST25” (524,205 bytes; created Apr. 16, 2010) are incorporated by reference herein in their entireties.

### FIELD OF THE INVENTION

The present invention relates to compositions and methods of their use for regulating nucleic acid expression.

### BACKGROUND OF THE INVENTION

In the last decade, progress has been made at an exponential rate in the development of vectors for gene therapy. The capability of efficient gene transfer to numerous target cells and tissues has led to an accumulation of preclinical data demonstrating potential efficacy in a broad range of animal models of human diseases [1-3]. As gene therapy studies advance from bench to bedside, proper transgene expression is being recognized as an important part of the research to achieve therapeutic effect and ensure safety in many gene therapy strategies [4-7]. Current regulation systems for controlling transgene expression typically require use of a special promoter and co-delivery of a cassette expressing a trans-activator protein. While these systems have been proven effective, the requirements prohibit differential regulation of multiple transgenes. Additionally, the requirement of co-delivering a trans-activator adds significantly to the payload required of the chosen gene transfer vector. The concern of payload is exacerbated for adeno-associated virus (AAV), which has a packaging limit of less than 5 kb [8].

The present invention provides a multiple gene expression regulation system without such drawbacks, based on regulation of splicing events (e.g., alternative splicing).

Alternative splicing is the differential selection of which exons will be included in a mature transcript during the process of pre-messenger RNA (pre-mRNA) splicing [32-35]. This mechanism is likely to be the most important engine driving the diverse array of proteins observed in cells. Alternative splicing can be divided into four general categories: (1) The simplest form of alternative splicing is a choice to remove or not to remove an intron (FIG. 1A); (2) The alternative use of 5′ splice sites, which will change the length of an exon (FIG. 1B); (3) The alternative use of 3′ splice sites, which will also change the length of an exon by extending the 3′ border of the exon (FIG. 1C); and (4) A more complex yet very frequent mode of alternative splicing is a choice between exon inclusion and exon skipping (FIG. 1D). In its simplest form, this choice involves one alternatively used exon in between two exons that are constitutively included. One of the main features of alternative splicing is that weak splice sites are usually found on alternative exons; either a weak 3′ splice site, or a weak 5′ splice site [32-35]. Alternative splicing via exon inclusion, the fourth category of alternative splicing mentioned above, was discovered to be the basis for human genetic diseases such as some cases of β-thalassemia [36-40], cystic fibrosis [41, 42], and others. These types of diseases are caused by mutations within introns that create novel splice sites. The mutant splice site, in conjunction with a nearby cryptic splice site, creates an aberrant alternative exon which is included into the mature message [36-42]. Inclusion of the aberrant exon(s) typically leads to synthesis of a non-functional protein or one with altered function. To treat this type of disease caused by inclusion of aberrant exons, exon skipping has been shown to be an effective strategy [9-12].

Important to this strategy, in some embodiments, is the use of anti-sense oligonucleotides (ASO or AON), which are designed to target the alternative splice sites. Hybridization of the ASO to the target splice site inhibited the inclusion and induced the skipping of the aberrant exon (9-12). As a result, the pre-mRNA is correctly spliced, leading to synthesis of a functional protein. The present invention employs this strategy in a unique way for controlling production of one or more functional exogenous proteins, peptides or RNA, e.g., to impart a therapeutic benefit.

Thus the present invention overcomes previous shortcomings in the art by providing improved compositions and methods for controlled expression of one or more exogenous or heterologous transgenes.

### SUMMARY OF THE INVENTION

In one aspect, the present invention provides a cell comprising a first heterologous nucleic acid construct and a second heterologous nucleic acid construct, wherein each of said first and second heterologous nucleic acid constructs comprises: A. a first nucleotide sequence encoding a nucleotide sequence of interest (NOI); and B. a second intronic nucleotide sequence comprising: i) a first set of splice elements defining a first intron that is removed by splicing to produce a first RNA molecule corresponding to said first nucleotide sequence in the absence of activity at a second set of splice elements; and ii) the second set of splice elements defining a second intron different from said first intron, wherein said second intron is removed by splicing to produce no RNA molecule and/or a second RNA molecule that does not correspond to said first nucleotide sequence, wherein said first nucleotide sequence of each of said first and second heterologous nucleic acid constructs is different from one another and wherein said second intronic nucleotide sequence of each of said first and second heterologous nucleic acid constructs is different from one another.

In various aspects of the invention, the second intronic nucleotide sequence of at least one of said first or second heterologous nucleic acid constructs of the cell of this invention can be the nucleotide sequence of any of SEQ ID NOS:1-242 as set forth herein, singly or in multiples and in any combination relative to one another and relative to the heterologous nucleic acid constructs of this invention.

A further aspect of the present invention is an isolated nucleic acid comprising: A) a first nucleotide sequence encoding a nucleotide sequence of interest (NOI); and B) a second intronic nucleotide sequence that can be:

a1) the nucleotide sequence of SEQ ID NO:92 (S0 257 by intron);

b1) the nucleotide sequence of SEQ ID NO:2 (S0−GT);

c1) the nucleotide sequence of SEQ ID NO:1 (S0−CT);

d1) the nucleotide sequence of SEQ ID NO:4 (S0−GT+CT);

e1) the nucleotide sequence of SEQ ID NO:3 (S1);

f1) the nucleotide sequence of SEQ ID NO:5 (S1+CT);

g1) the nucleotide sequence of SEQ ID NO:6 (M3);

h1) the nucleotide sequence of SEQ ID NO:7 (M3+CT);

i1) the nucleotide sequence of SEQ ID NO:8 (M6);

j1) the nucleotide sequence of SEQ ID NO:9 (M6+CT);

k1) the nucleotide sequence of SEQ ID NO:14 (M3+S1);

l1) the nucleotide sequence of SEQ ID NO:16 (M3+S1+CT);

m1) the nucleotide sequence of SEQ ID NO:15 (M6+S1);

n1) the nucleotide sequence of SEQ ID NO:17 (M6+S1+CT);

o1) the nucleotide sequence of SEQ ID NO:22, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

p1) the nucleotide sequence of SEQ ID NO:23, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

q1) the nucleotide sequence of SEQ ID NO:24, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;

r1) the nucleotide sequence of SEQ ID NO:25, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;

s1) the nucleotide sequence of SEQ ID NO:26, which comprises the sequence X₁GX₃GX₅X₆X₇AGGTAAGTX₁₄ (SEQ ID NO:326), wherein X of any of X₁, X₃, X₅-X₇ and X₁₄ can be A, C, T or G, in any combination;

t1) the nucleotide sequence of SEQ ID NO:27, which comprises the sequence X₁GX₃GX₅X₆X₇AGGTAAGTX₁₄ (SEQ ID NO:326), wherein X of any of X₁, X₃, X₅-X₇ and X₁₄ can be A, C, T or G, in any combination;

u1) the nucleotide sequence of SEQ ID NO:28, which comprises the sequence X₁X₂X₃X₄X₅X₆AAGGTAATAG (SEQ ID NO:327), wherein X of any of X₁,-X₆ can be A, C, T or G, in any combination;

v1) the nucleotide sequence of SEQ ID NO:29, which comprises the sequence X₁X₂X₃X₄X₅X₆AAGGTAATAG (SEQ ID NO:327), wherein X of any of X₁,-X₆ can be A, C, T or G, in any combination;

w1) the nucleotide sequence of SEQ ID NO:30, which comprises the sequence X₁GX₃X₄X₅X₆X₇AGGTAATAG (SEQ ID NO:328), wherein X of any of X₁ and X₃-X₇ can be A, C, T or G, in any combination;

x1) the nucleotide sequence of SEQ ID NO:31, which comprises the sequence X₁GX₃X₄X₅X₆X₇AGGTAATAG (SEQ ID NO:328), wherein X of any of X₁, and X₃-X₇ can be A, C, T or G, in any combination;

y1) the nucleotide sequence of SEQ ID NO:32, which comprises the sequence X₁X₂GX₄X₅X₆X₇AGGTAATAG (SEQ ID NO:329), wherein X of any of X₁, X₂ and X₄-X₇ can be A, C, T or G, in any combination;

z1) the nucleotide sequence of SEQ ID NO:33, which comprises the sequence X₁X₂GX₄X₅X₆X₇AGGTAATAG (SEQ ID NO:329), wherein X of any of X₁, X₂ and X₄-X₇ can be A, C, T or G, in any combination;

a2) the nucleotide sequence of SEQ ID NO:34, which comprises the sequence X₁X₂X₃GX₅X₆X₇AGGTAATAG (SEQ ID NO:330), wherein X of any of X₁-X₃ and X₅-X₇ can be A, C, T or G, in any combination;

b2) the nucleotide sequence of SEQ ID NO:35, which comprises the sequence X₁X₂X₃GX₅X₈X₇AGGTAATAG (SEQ ID NO:330), wherein X of any of X₁-X₃ and X₅-X₇ can be A, C, T or G, in any combination;

c2) the nucleotide sequence of SEQ ID NO:36, which comprises the sequence X₁X₂X₃X₄TX₆X₇AGGTAATAG (SEQ ID NO:331), wherein X of any of X₁-X₄ and X₆-X₇ can be A, C, T or G, in any combination;

d2) the nucleotide sequence of SEQ ID NO:37, which comprises the sequence X₁X₂X₃X₄TX₈X₇AGGTAATAG (SEQ ID NO:331), wherein X of any of X₁-X₄ and X₆-X₇ can be A, C, T or G, in any combination;

e2) the nucleotide sequence of SEQ ID NO:38, which comprises the sequence X₁GGX₄X₅X₈X₇X₈GGTAATAG (SEQ ID NO:332), wherein X of any of X₁ and X₄-X₈ can be A, C, T or G, in any combination;

f2) the nucleotide sequence of SEQ ID NO:39, which comprises the sequence X₁GGX₄X₅X₈X₇X₈GGTAATAG (SEQ ID NO:332), wherein X of any of X₁ and X₄-X₈ can be A, C, T or G, in any combination;

g2) the nucleotide sequence of SEQ ID NO:40, which comprises the sequence X₁X₂GGX₅X₈X₇X₈GGTAATAG (SEQ ID NO:333), wherein X of any of X₁, X₂ and X₅-X₈ can be A, C, T or G, in any combination;

h2) the nucleotide sequence of SEQ ID NO:41, which comprises the sequence X₁X₂GGX₅X₈X₇X₈GGTAATAG (SEQ ID NO:333), wherein X of any of X₁, X₂ and X₅-X₈ can be A, C, T or G, in any combination;

i2) the nucleotide sequence of SEQ ID NO:42, which comprises the sequence X₁X₂X₃GTX₈X₇X₈GGTAATAG (SEQ ID NO:334), wherein X of any of X₁-X₃ and X₈-X₈ can be A, C, T or G, in any combination;

j2) the nucleotide sequence of SEQ ID NO:43, which comprises the sequence X₁X₂X₃GTX₈X₇X₈GGTAATAG (SEQ ID NO:334), wherein X of any of X₁-X₃ and X₈-X₈ can be A, C, T or G, in any combination;

k2) the nucleotide sequence of SEQ ID NO:44, which comprises the sequence X₁X₂X₃X₄TTX₇X₈GGTAATAG (SEQ ID NO:335), wherein X of any of X₁-X₄ and X₇-X₈ can be A, C, T or G, in any combination;

l2) the nucleotide sequence of SEQ ID NO:45, which comprises the sequence X₁X₂X₃X₄TTX₇X₈GGTAATAG (SEQ ID NO:335), wherein X of any of X₁-X₄ and X₇-X₈ can be A, C, T or G, in any combination;

m2) the nucleotide sequence of SEQ ID NO:46, which comprises the sequence X₁X₂X₃X₄X₅TAX₈GGTAATAG (SEQ ID NO:336), wherein X of any of X₁-X₅ and X₈ can be A, C, T or G, in any combination;

n2) the nucleotide sequence of SEQ ID NO:47, which comprises the sequence X₁X₂X₃X₄X₅TAX₈GGTAATAG (SEQ ID NO:336), wherein X of any of X₁-X₅ and X₅ can be A, C, T or G, in any combination;

o2) the nucleotide sequence of SEQ ID NO:48, which comprises the sequence X₁X₂X₃X₄X₅X₆AAGGTAAGTG (SEQ ID NO:337), wherein X of any of X₁-X₆ can be A, C, T or G, in any combination;

p2) the nucleotide sequence of SEQ ID NO:49, which comprises the sequence X₁X₂X₃X₄X₅X₆AAGGTAAGTG (SEQ ID NO:337), wherein X of any of X₁-X₆ can be A, C, T or G, in any combination;

q2) the nucleotide sequence of SEQ ID NO:50, which comprises the sequence X₁GX₃X₄X₅X₆X₇AGGTAAGTG (SEQ ID NO:338), wherein X of any of X₁ and X₃-X₇ can be A, C, T or G, in any combination;

r2) the nucleotide sequence of SEQ ID NO:51, which comprises the sequence X₁GX₃X₄X₅X₆X₇AGGTAAGTG (SEQ ID NO:338), wherein X of any of X₁ and X₃-X₇ can be A, C, T or G, in any combination;

s2) the nucleotide sequence of SEQ ID NO:52, in any combination, which comprises the sequence X₁X₂GX₄X₅X₆X₇AGGTAAGTG (SEQ ID NO:339), wherein X of any of X₁-X₂ and X₄-X₇ can be A, C, T or G;

t2) the nucleotide sequence of SEQ ID NO:53, which comprises the sequence X₁X₂GX₄X₅X₆X₇AGGTAAGTG (SEQ ID NO:339), wherein X of any of X₁-X₂ and X₄-X₇ can be A, C, T or G, in any combination;

u2) the nucleotide sequence of SEQ ID NO:54, which comprises the sequence X₁X₂X₃GX₅X₆X₇AGGTAAGTG (SEQ ID NO:340), wherein X of any of X₁-X₃ and X₅-X₇ can be A, C, T or G, in any combination;

v2) the nucleotide sequence of SEQ ID NO:55, which comprises the sequence X₁X₂X₃GX₅X₆X₇AGGTAAGTG (SEQ ID NO:340), wherein X of any of X₁-X₃ and X₅-X₇ can be A, C, T or G, in any combination;

w2) the nucleotide sequence of SEQ ID NO:56, which comprises the sequence X₁X₂X₃X₄TX₆X₇AGGTAAGTG (SEQ ID NO:341), wherein X of any of X₁-X₄ and X₆-X₇ can be A, C, T or G, in any combination;

x2) the nucleotide sequence of SEQ ID NO:57, which comprises the sequence X₁X₂X₃X₄TX₆X₇AGGTAAGTG (SEQ ID NO:341), wherein X of any of X₁-X₄ and X₆-X₇ can be A, C, T or G, in any combination;

y2) the nucleotide sequence of SEQ ID NO:58, which comprises the sequence X₁GGX₄X₅X₆X₇X₈GGTAAGTG (SEQ ID NO:342), wherein X of any of X₁ and X₄-X₈ can be A, C, T or G, in any combination;

z2) the nucleotide sequence of SEQ ID NO:59, which comprises the sequence X₁GGX₄X₅X₈X₇X₈GGTAAGTG (SEQ ID NO:342), wherein X of any of X₁ and X₄-X₈ can be A, C, T or G, in any combination;

a3) the nucleotide sequence of SEQ ID NO:60, which comprises the sequence X₁X₂GGX₅X₈X₇X₈GGTAAGTG (SEQ ID NO:343), wherein X of any of X₁-X₂ and X₅-X₈ can be A, C, T or G, in any combination;

b3) the nucleotide sequence of SEQ ID NO:61, which comprises the sequence X₁X₂GGX₅X₈X₇X₈GGTAAGTG (SEQ ID NO:343), wherein X of any of X₁-X₂ and X₅-X₈ can be A, C, T or G, in any combination;

c3) the nucleotide sequence of SEQ ID NO:62, which comprises the sequence X₁X₂X₃GTX₈X₇X₈GGTAAGTG (SEQ ID NO:344), wherein X of any of X₁-X₂ and X₅-X₈ can be A, C, T or G, in any combination;

d3) the nucleotide sequence of SEQ ID NO:63, which comprises the sequence X₁X₂X₃GTX₆X₇X₈GGTAAGTG (SEQ ID NO:344), wherein X of any of X₁-X₃ and X₈-X₈ can be A, C, T or G, in any combination;

e3) the nucleotide sequence of SEQ ID NO:64, which comprises the sequence X₁X₂X₃X₄TTX₇X₈GGTAAGTG (SEQ ID NO:345), wherein X of any of X₁-X₄ and X₇-X₈ can be A, C, T or G, in any combination;

f3) the nucleotide sequence of SEQ ID NO:65, which comprises the sequence X₁X₂X₃X₄TTX₇X₈GGTAAGTG (SEQ ID NO:345), wherein X of any of X₁-X₄ and X₇-X₈ can be A, C, T or G, in any combination;

g3) the nucleotide sequence of SEQ ID NO:66, which comprises the sequence X₁X₂X₃X₄X₅TAX₈GGTAATGTG (SEQ ID NO:346), wherein X of any of X₁-X₅ and X₈ can be A, C, T or G, in any combination;

h3) the nucleotide sequence of SEQ ID NO:67, which comprises the sequence X₁X₂X₃X₄X₅TAX₈GGTAATGTG (SEQ ID NO:346), wherein X of any of X₁-X₅ and X₈ can be A, C, T or G, in any combination;

i3) the nucleotide sequence of SEQ ID NO:68, which comprises the sequence AGCGAATAGGTAATAC (SEQ ID NO:347);

j3) the nucleotide sequence of SEQ ID NO:69, which comprises the sequence AGCGAATAGGTAATAC (SEQ ID NO:347);

k3) the nucleotide sequence of SEQ ID NO:70, which comprises the sequence CGAGGGCAGGTAATAA (SEQ ID NO:348);

l3) the nucleotide sequence of SEQ ID NO:71, which comprises the sequence CGAGGGCAGGTAATAA (SEQ ID NO:348);

m3) the nucleotide sequence of SEQ ID NO:72, which comprises the sequence GGTGCCGAGGTAATAT (SEQ ID NO:349); n3) the nucleotide sequence of SEQ ID NO:73, which comprises the sequence GGTGCCGAGGTAATAT (SEQ ID NO:349);

o3) the nucleotide sequence of SEQ ID NO:74, which comprises the sequence GGTGACTAGGTAATAC (SEQ ID NO:350);

p3) the nucleotide sequence of SEQ ID NO:75, which comprises the sequence GGTGACTAGGTAATAC (SEQ ID NO:350);

q3) the nucleotide sequence of SEQ ID NO:76, which comprises the sequence CGAGGGCAGGTAATAT (SEQ ID NO:351);

r3) the nucleotide sequence of SEQ ID NO:77, which comprises the sequence to CGAGGGCAGGTAATAT (SEQ ID NO:351);

s3) the nucleotide sequence of SEQ ID NO:78, which comprises the sequence AGCGCAGAGGTAATAA (SEQ ID NO:352);

t3) the nucleotide sequence of SEQ ID NO:79, which comprises the sequence AGCGCAGAGGTAATAA (SEQ ID NO:352);

u3) the nucleotide sequence of SEQ ID NO:80, which comprises the sequence AGCGAATAGGTAAGTC (SEQ ID NO:353);

v3) the nucleotide sequence of SEQ ID NO:81, which comprises the sequence AGCGAATAGGTAAGTC (SEQ ID NO:353);

w3) the nucleotide sequence of SEQ ID NO:82, which comprises the sequence CGAGGGCAGGTAAGTA (SEQ ID NO:354);

x3) the nucleotide sequence of SEQ ID NO:83, which comprises the sequence CGAGGGCAGGTAAGTA (SEQ ID NO:354);

y3) the nucleotide sequence of SEQ ID NO:84, which comprises the sequence GGTGCCGAGGTAAGTT (SEQ ID NO:355);

z3) the nucleotide sequence of SEQ ID NO:85, which comprises the sequence GGTGCCGAGGTAAGTT (SEQ ID NO:355);

a4) the nucleotide sequence of SEQ ID NO:86, which comprises the sequence GGTGACTAGGTAAGTC (SEQ ID NO:356);

b4) the nucleotide sequence of SEQ ID NO:87, which comprises the sequence GGTGACTAGGTAAGTC (SEQ ID NO:356);

c4) the nucleotide sequence of SEQ ID NO:88, which comprises the sequence CGAGGGCAGGTAAGTT (SEQ ID NO:357);

d4) the nucleotide sequence of SEQ ID NO:89, which comprises the sequence CGAGGGCAGGTAAGTT (SEQ ID NO:357);

e4) the nucleotide sequence of SEQ ID NO:90, which comprises the sequence AGCGCAGAGGTAAGTA (SEQ ID NO:358);

f4) the nucleotide sequence of SEQ ID NO:91, which comprises the sequence AGCGCAGAGGTAAGTA (SEQ ID NO:358);

g4) the nucleotide sequence of SEQ ID NO:10);

h4) the nucleotide sequence of SEQ ID NO:11;

i4) the nucleotide sequence of SEQ ID NO:12;

j4) the nucleotide sequence of SEQ ID NO:13;

k4) the nucleotide sequence of SEQ ID NO:18;

l4) the nucleotide sequence of SEQ ID NO:20;

m4) the nucleotide sequence of SEQ ID NO:19;

n4) the nucleotide sequence of SEQ ID NO:21; and

o4) any combination of a1 through n4 above.

In particular aspects of the invention, the nucleotide sequence of interest (NOI) of the first nucleotide sequence can be a) a nucleotide sequence encoding a protein or peptide; b) a nucleotide sequence encoding a product having activity as an interfering RNA (e.g., siRNA, microRNA, shRNA); c) a nucleotide sequence encoding a product having enzymatic activity as an RNA; d) a nucleotide sequence encoding a ribozyme; e) a nucleotide sequence encoding an antisense sequence; f) a nucleotide sequence encoding a small nuclear RNA (snRNA); and g) any combination of (a)-(f) above.

Further provided in this invention is a method of producing a functional RNA encoded by said first nucleotide sequence of said first heterologous nucleic acid construct in a cell of this invention, comprising: introducing into said cell a blocking oligonucleotide and/or small molecule that blocks a member of said second set of splice elements of said second intronic nucleotide sequence of said first heterologous nucleic acid construct, thereby producing the functional RNA encoded by the first nucleotide sequence of said first heterologous nucleic acid construct in said cell.

Also provided herein is a method of producing a functional RNA encoded by said first nucleotide sequence of said second heterologous nucleic acid construct in said cell of this invention, comprising: introducing into said cell a blocking oligonucleotide and/or small molecule that blocks a member of said second set of splice elements of said second intronic nucleotide sequence of said second heterologous nucleic acid construct, thereby producing the functional RNA encoded by the first nucleotide sequence of said second heterologous nucleic acid construct in said cell.

An additional aspect of this invention is a method of producing a first functional RNA encoded by a first nucleotide sequence of a first heterologous nucleic acid construct and producing a second functional RNA encoded by a first nucleotide sequence of a second heterologous nucleic acid construct, in a cell of this invention comprising said first heterologous nucleic acid construct and said second heterologous nucleic acid construct, wherein said first functional RNA and said second functional RNA are different from each other, comprising: a) introducing into said cell a first blocking oligonucleotide and/or first small molecule that blocks a member of said second set of splice elements of said second intronic nucleotide sequence of said first heterologous nucleic acid construct, thereby producing said first functional RNA encoded by said first nucleotide sequence of said first heterologous nucleic acid construct in said cell, and b) introducing into said cell a second blocking oligonucleotide and/or second small molecule that blocks a member of said second set of splice elements of said second intronic nucleotide sequence of said second heterologous nucleic acid construct, thereby producing said second functional RNA encoded by said first nucleotide sequence of said second heterologous nucleic acid construct in said cell, wherein said second intronic nucleotide sequence of said first heterologous nucleic acid construct and said second intronic nucleotide sequence of said second heterologous nucleic acid construct are different from each other and wherein said first blocking oligonucleotide and/or first small molecule and said second blocking oligonucleotide and/or second small molecule are different from each other.

Additionally provided herein is a method for producing a functional RNA encoded by said first nucleotide sequence of an isolated nucleic acid of this invention, comprising contacting a blocking oligonucleotide and/or small molecule with the isolated nucleic acid under conditions that permit splicing, wherein the blocking oligonucleotide and/or small molecule blocks a member of said second set of splice elements of said second intronic nucleotide sequence, thereby producing the functional RNA encoded by said first nucleotide sequence.

The foregoing and other objects and aspects of the present invention are explained in detail in the specification set forth below.

### BRIEF DESCRIPTION OF THE DRAWINGS

FIGS. 1A-D. Four types of splicing of pre-messenger RNAs (see, e.g., ref. 32). Alternatively recognized sequences are indicated with dashed lines.

FIGS. 2A-B. Regulation of GFP expression by controlling alternative splicing. A) Schematic representation of the mechanism. The IVS2-654 intron mostly undergoes aberrant splicing (AS), resulting in inclusion of an alternatively used exon and thus synthesis of a non-functional protein. However, in the presence of oligonucleotides complementary to the 5′ alternative splice site as represented by the grey bar, aberrant splicing is inhibited and correct splicing (CS) becomes the major pathway. As a result, a functional protein is synthesized, a GFP protein in this case. B) Splicing pattern of GFP mRNA. mRNAs extracted from the above treated cells were subjected to an RT-PCR assay as described herein for characterizing the alternative splicing. The RT-PCR products were separated on an 8% PAGE gel. Cells that were later used in lanes 1, 4, 5, 8, 9 and 12 were mock transfected. Cells that were later used in lanes 2, 6 and 10 were transfected with the control LAN654M. Cells that were later used in lanes 3, 7 and 11 were transfected with LAN654.

FIGS. 3A-B. Regulation of AAT expression by controlling alternative splicing. A) Splicing pattern of AAT mRNA. Plasmid pAAV654AAT was transfected into 293 cells which were also treated with no ASO, LNA654M or LNA654. mRNAs from the cells were extracted and subjected to an RT-PCR assay for characterizing the alternative splicing. The RT-PCR products were separated on an 8% PAGE gel. B) Regulated AAT expression in vivo. AAV654AAT vectors were administered into mice via portal vein injection (n=5). At 6 weeks post infection, one group of the mice received 2 consecutive days of LNA654 injection and the other received no LNA654. Blood samples were collected at various days post ASO injection as indicated. AAT levels in the serum were then assayed as described herein.

FIGS. 4A-B. Effect of intron insertion site on the induction level of transgene expression. A) Schematic illustration of the intron insertion sites in the luciferase expression cassette. The IVS2-654 intron was inserted into sites A-D and F within the luciferase expression cassette as described herein to enable regulation of the transgene expression. B) Induction levels of luciferase expression for the intron insertion constructs. Plasmids A-D and F were transfected into 293 cells which were also treated with LNA654M or LNA654 as indicated. The levels of luciferase transgene expressed were assayed 24 hours after the treatments and expressed as total relative light units (RLU) per 24-well. The induction level, was calculated by dividing the amount of luciferase expressed in the presence of LNA654 by that in the presence of LNA654M. C) Splicing pattern of luciferase mRNA. mRNAs extracted from an identical set of cells as described above were subjected to an RT-PCR assay for characterizing the alternative splicing. The RT-PCR products were separated on an 8% PAGE gel. Cells that were later used in lanes 1, 3, 5, 7 and 9 were transfected with the control LAN654M. Cells that were later used in lanes 2, 4, 6, 8 and 10 were transfected with LAN654. As a positive control, cells that were later used in lane 12 were transfected without any ASO and with a construct containing the luciferase expression cassette inserted with a wild type IVS2 intron at site B. As a negative control, cells that were later used in lane 11 were mock transfected and without any ASO treatment. All primers used for the RT-PCR assay as well as the expected sizes of the assay products are listed in Table I. Arrow heads point to PCR products from alternative splicing (AS). The faster migrating bands are from correct splicing (CS).

FIGS. 5A-B. Effect of distance between introns on the control of luciferase expression. A. Data of luciferase expression for various constructs. B. The relationship between intron distance and induction level.

### DETAILED DESCRIPTION OF THE INVENTION

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

The present invention is based on the unexpected discovery that expression of a nucleic acid or multiple nucleic acids (e.g., 2, 3, 4, 5, 6, 7, 8, 9, 10, etc.), such as a heterologous nucleotide sequence or multiple heterologous nucleotide sequences, can be closely regulated, e.g., in vivo, at the post-transcriptional level. Such regulation is based on the selective splicing of different introns operatively associated with the nucleic acid, according to the presence or absence of an oligonucleotide, small molecule and/or other compound that selectively blocks splicing activity at specific splicing sites. Such regulation of heterologous nucleotide sequence expression can be further refined and controlled by employing the different introns described herein, which comprise mutations that allow, e.g., for more controlled regulation of a specific nucleotide sequence of interest, as well as for the controlled expression of multiple different heterologous nucleotide sequences of interest.

Thus, in one embodiment, the present invention provides a cell comprising a first heterologous nucleic acid construct and a second heterologous nucleic acid construct, wherein each of said first and second heterologous nucleic acid constructs comprises: A. a first nucleotide sequence encoding a nucleotide sequence of interest (NOI); and B. a second intronic nucleotide sequence comprising: i) a first intron defined by a first set of splice elements that is removed by splicing to produce a first RNA molecule encoded by said first nucleotide sequence (e.g., a functional RNA molecule) under conditions whereby removal of a second intron defined by a second set of splice elements is prevented (e.g., in the absence of activity at the second set of splice elements); and

ii) the second intron defined by the second set of splice elements wherein said second intron is different from said first intron, and wherein under conditions whereby removal of said second intron is not prevented (e.g., in the absence of a blocking oligonucleotide and/or blocking molecule) and said second intron is removed by splicing, no RNA molecule and/or a second RNA molecule that is not encoded by said first nucleotide sequence (e.g., a nonfunctional RNA molecule) is produced, and further wherein each said NOI of said first nucleotide sequence of each of said respective first and second heterologous nucleic acid constructs is different from one another and wherein each said second intronic nucleotide sequence of each of said respective first and second heterologous nucleic acid constructs is different from one another.

In some embodiments of the cell of this invention, the first and second heterologous nucleic acid constructs can be present in and/or introduced into the cell as separate nucleic acid constructs and in some embodiments, the first and second heterologous nucleic acid constructs can be present in and/or introduced into the cell as a single nucleic acid construct (e.g., combined to be present in the same construct). As used herein, a “nucleic acid construct” is a recombinant nucleic acid molecule comprising at least one first nucleotide sequence and at least one second intronic nucleotide sequence, along with operably associated regulatory elements that allow for expression of the first nucleotide sequence(s) and differential splicing of the various intron sequences present in the second intronic nucleotide sequence.

A cell comprising a heterologous nucleic acid construct of this invention is any cell (e.g., an isolated cell; a transformed cell, etc.) and such a cell can be present in vitro or in vivo. In addition, although the embodiments described above recite a first heterologous nucleic acid construct and a second heterologous nucleic acid construct, it is well within the scope of this invention for a cell of this invention to comprise more than one or two heterologous nucleic acid constructs and indeed such a cell comprising multiple heterologous nucleic acid constructs (e.g., a second, third, fourth, fifth, sixth, seventh, eighth, ninth, tenth, etc) is an inventive aspect of this invention, particularly in embodiments wherein the multiple heterologous nucleic acid constructs comprise first nucleotide sequences that encode a variety of different proteins, peptides and/or RNAs, the expression of each of which can be differentially regulated according to the methods of this invention as described herein.

Furthermore, the first nucleotide sequence of a heterologous nucleic acid construct of this invention and/or of an isolated nucleic acid of this invention can encode a nucleotide sequence of interest (NOI) that can be, but is not limited to, a) a nucleotide sequence encoding a protein or peptide; b) a nucleotide sequence encoding a product having activity as an interfering RNA (e.g., siRNA, shRNA, microRNA); c) a nucleotide sequence encoding a product having enzymatic activity as an RNA; d) a nucleotide sequence encoding a ribozyme; e) a nucleotide sequence encoding an antisense sequence; f) a nucleotide sequence encoding a small nuclear RNA (snRNA); and g) any combination of (a)-(f) above. Examples of such proteins, peptides, RNA molecules, ribozymes, etc. are provided herein and are also well known in the art.

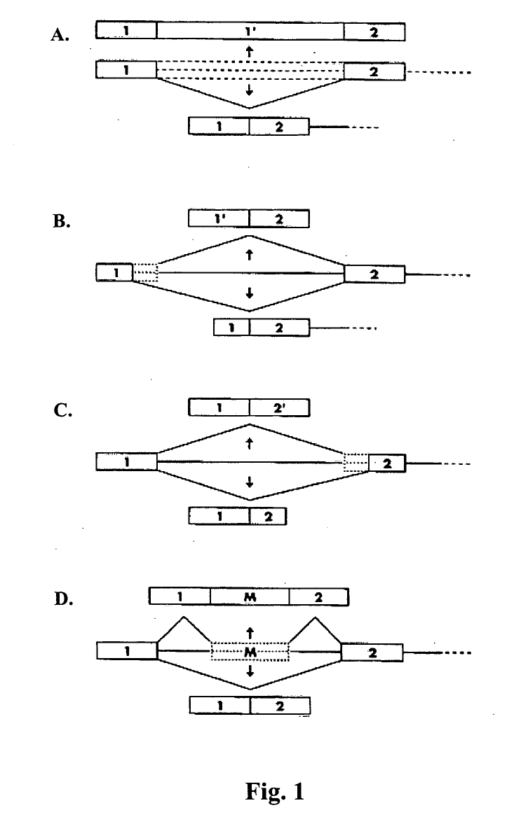

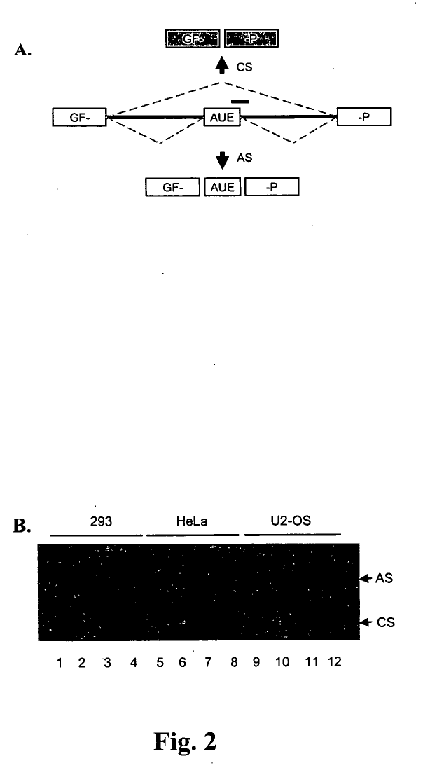

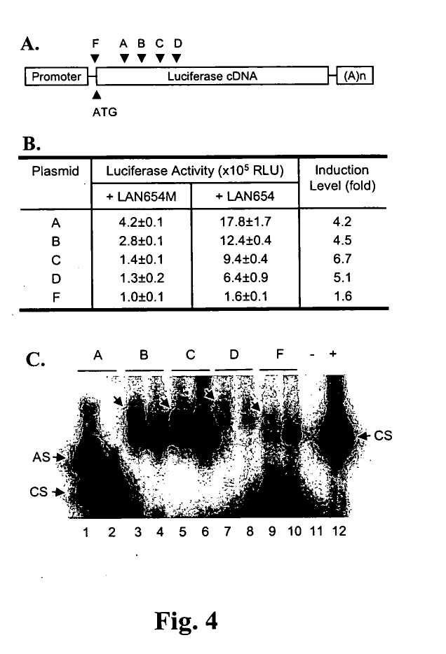

It will be understood by those of ordinary skill in the art that the second intronic nucleotide sequence of this invention has the function and structure of an intron sequence or multiple intron sequences. Such an intron sequence can be positioned at any site in a first nucleotide sequence singly or in multiples and the intron sequence can be the same or different in any combination in any given first nucleotide sequence. For examples of the different configurations of the intron sequence(s) of this invention, see FIGS. 1A-D, FIGS. 2A and 4A provided herein. In some embodiments of the invention, the “first intron” is the “correct” intron sequence that is to be spliced out, resulting in no remaining intron sequences and also resulting in the formation of a functional RNA (e.g., an RNA that is directly functional or is functional for translation into a functional protein or peptide, as described herein). Also in some embodiments of this invention, the “second intron” comprises one or more intron sequences that are “incorrect” or “alternative” or “aberrant” intron sequences, in that splicing events that remove the second intron(s) do not result in the formation of a functional RNA (e.g., no RNA is produced or an RNA is produced upon splicing out of the second intron which is not functional directly as an RNA or is not functional for translation into a desired protein or peptide, such as that encoded by the first nucleotide sequence of this invention). In some embodiments, when production of the functional first RNA encoded by the first nucleotide sequence is desired, an oligonucleotide and/or small molecule and/or other blocking agent is delivered into the cell (or is activated if already present in the cell in an inactive form) to block splicing events at the second set of splice elements that define the second intron, resulting in splicing activity at the first set of splice elements that define the first intron, thereby removing the first intron and producing a functional first RNA.

As described herein the second intronic nucleotide sequence is “operatively associated” with the first nucleotide sequence of this invention. As used here, this means that the second intronic nucleotide sequence is located within and/or in proximity to the first nucleotide sequence at a single location or at multiple locations such that the second intronic nucleotide sequence functions as an intron that is spliced out 1) to produce a functional first RNA when splicing activity occurs at the first set of splice elements defining the first intron; or 2) to produce no RNA or an RNA that is not the functional RNA encoded by the first nucleotide sequence when splicing activity occurs at the second set of splice elements. As described herein, a first nucleotide sequence of this invention can comprise multiple second intronic nucleotide sequences, each comprising a second set of splice elements, each of which can be the same or different from one another in any combination.

In some embodiments of the cell of this invention, the second intronic nucleotide sequence of at least one of said first and/or second heterologous nucleic acid constructs can be, but is not limited to,

a1) the nucleotide sequence of SEQ ID NO:92;

b1) the nucleotide sequence of SEQ ID NO:2;

c1) the nucleotide sequence of SEQ ID NO:1;

d1) the nucleotide sequence of SEQ ID NO:4;

e1) the nucleotide sequence of SEQ ID NO:3;

f1) the nucleotide sequence of SEQ ID NO:5;

g1) the nucleotide sequence of SEQ ID NO:6;

h1) the nucleotide sequence of SEQ ID NO:7;

i1) the nucleotide sequence of SEQ ID NO:8;

j1) the nucleotide sequence of SEQ ID NO:9;

k1) the nucleotide sequence of SEQ ID NO:14;

l1) the nucleotide sequence of SEQ ID NO:16;

m1) the nucleotide sequence of SEQ ID NO:15;

n1) the nucleotide sequence of SEQ ID NO:17;

o1) the nucleotide sequence of SEQ ID NO:22, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

p1) the nucleotide sequence of SEQ ID NO:23, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

q1) the nucleotide sequence of SEQ ID NO:24, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;

r1) the nucleotide sequence of SEQ ID NO:25, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;

s1) the nucleotide sequence of SEQ ID NO:26, which comprises the sequence X₁GX₃GX₅X₆X₇AGGTAAGTX₁₄ (SEQ ID NO:326), wherein X of any of X₁, X₃, X₅-X₇ and X₁₄ can be A, C, T or G, in any combination;

t1) the nucleotide sequence of SEQ ID NO:27, which comprises the sequence X₁GX₃GX₅X₆X₇AGGTAAGTX₁₄ (SEQ ID NO:326), wherein X of any of X₁, X₃, X₅-X₇ and X₁₄ can be A, C, T or G, in any combination;

u1) the nucleotide sequence of SEQ ID NO:28, which comprises the sequence X₁X₂X₃X₄X₅X₆AAGGTAATAG (SEQ ID NO:327), wherein X of any of X₁,-X₆ can be A, C, T or G, in any combination;

v1) the nucleotide sequence of SEQ ID NO:29, which comprises the sequence X₁X₂X₃X₄X₅X₆AAGGTAATAG (SEQ ID NO:327), wherein X of any of X₁,-X₆ can be A, C, T or G, in any combination;

w1) the nucleotide sequence of SEQ ID NO:30, which comprises the sequence X₁GX₃X₄X₅X₆X₇AGGTAATAG (SEQ ID NO:328), wherein X of any of X₁ and X₃-X₇ can be A, C, T or G, in any combination;

x1) the nucleotide sequence of SEQ ID NO:31, which comprises the sequence X₁GX₃X₄X₅X₈X₇AGGTAATAG (SEQ ID NO:328), wherein X of any of X₁, and X₃-X₇ can be A, C, T or G, in any combination;

y1) the nucleotide sequence of SEQ ID NO:32, which comprises the sequence X₁X₂GX₄X₅X₆X₇AGGTAATAG (SEQ ID NO:329), wherein X of any of X₁, X₂ and X₄-X₇ can be A, C, T or G, in any combination;

z1) the nucleotide sequence of SEQ ID NO:33, which comprises the sequence X₁X₂GX₄X₅X₈X₇AGGTAATAG (SEQ ID NO:329), wherein X of any of X₁, X₂ and X₄-X₇ can be A, C, T or G, in any combination;

a2) the nucleotide sequence of SEQ ID NO:34, which comprises the sequence X₁X₂X₃GX₅X₆X₇AGGTAATAG (SEQ ID NO:330), wherein X of any of X₁-X₃ and X₅-X₇ can be A, C, T or G, in any combination;

b2) the nucleotide sequence of SEQ ID NO:35, which comprises the sequence X₁X₂X₃GX₅X₈X₇AGGTAATAG (SEQ ID NO:330), wherein X of any of X₁-X₃ and X₅-X₇ can be A, C, T or G, in any combination;

c2) the nucleotide sequence of SEQ ID NO:36, which comprises the sequence X₁X₂X₃X₄TX₈X₇AGGTAATAG (SEQ ID NO:331), wherein X of any of X₁-X₄ and X₆-X₇ can be A, C, T or G, in any combination;

d2) the nucleotide sequence of SEQ ID NO:37, which comprises the sequence X₁X₂X₃X₄TX₆X₇AGGTAATAG (SEQ ID NO:331), wherein X of any of X₁-X₄ and X₆-X₇ can be A, C, T or G, in any combination;

e2) the nucleotide sequence of SEQ ID NO:38, which comprises the sequence X₁GGX₄X₅X₈X₇X₈GGTAATAG (SEQ ID NO:332), wherein X of any of X₁ and X₄-X₈ can be A, C, T or G, in any combination;

f2) the nucleotide sequence of SEQ ID NO:39, which comprises the sequence X₁GGX₄X₅X₆X₇X₈GGTAATAG (SEQ ID NO:332), wherein X of any of X₁ and X₄-X₈ can be A, C, T or G, in any combination;

g2) the nucleotide sequence of SEQ ID NO:40, which comprises the sequence X₁X₂GGX₅X₆X₇X₈GGTAATAG (SEQ ID NO:333), wherein X of any of X₁, X₂ and X₅-X₈ can be A, C, T or G, in any combination;

h2) the nucleotide sequence of SEQ ID NO:41, which comprises the sequence X₁X₂GGX₅X₆X₇X₈GGTAATAG (SEQ ID NO:333), wherein X of any of X₁, X₂ and X₅-X₈ can be A, C, T or G, in any combination;

i2) the nucleotide sequence of SEQ ID NO:42, which comprises the sequence X₁X₂X₃GTX₆X₇X₈GGTAATAG (SEQ ID NO:334), wherein X of any of X₁-X₃ and X₆-X₈ can be A, C, T or G, in any combination;

j2) the nucleotide sequence of SEQ ID NO:43, which comprises the sequence X₁X₂X₃GTX₆X₇X₈GGTAATAG (SEQ ID NO:334), wherein X of any of X₁-X₃ and X₆-X₈ can be A, C, T or G, in any combination;

k2) the nucleotide sequence of SEQ ID NO:44, which comprises the sequence X₁X₂X₃X₄TTX₇X₈GGTAATAG (SEQ ID NO:335), wherein X of any of X₁-X₄ and X₇-X₈ can be A, C, T or G, in any combination;

l2) the nucleotide sequence of SEQ ID NO:45, which comprises the sequence X₁X₂X₃X₄TTX₇X₈GGTAATAG (SEQ ID NO:335), wherein X of any of X₁-X₄ and X₇-X₈ can be A, C, T or G, in any combination;

m2) the nucleotide sequence of SEQ ID NO:46, which comprises the sequence X₁X₂X₃X₄X₆TAX₈GGTAATAG (SEQ ID NO:336), wherein X of any of X₁-X₅ and X₈ can be A, C, T or G, in any combination;

n2) the nucleotide sequence of SEQ ID NO:47, which comprises the sequence X₁X₂X₃X₄X₆TAX₈GGTAATAG (SEQ ID NO:336), wherein X of any of X₁-Xₛ and X₅ can be A, C, T or G, in any combination;

o2) the nucleotide sequence of SEQ ID NO:48, which comprises the sequence X₁X₂X₃X₄X₆X₆AAGGTAAGTG (SEQ ID NO:337), wherein X of any of X₁-X₆ can be A, C, T or G, in any combination;

p2) the nucleotide sequence of SEQ ID NO:49, which comprises the sequence X₁X₂X₃X₄X₆X₆AAGGTAAGTG (SEQ ID NO:337), wherein X of any of X₁-X₆ can be A, C, T or G, in any combination;

q2) the nucleotide sequence of SEQ ID NO:50, which comprises the sequence X₁GX₃X₄X₆X₆X₇AGGTAAGTG (SEQ ID NO:338), wherein X of any of X₁ and X₃-X₇ can be A, C, T or G, in any combination;

r2) the nucleotide sequence of SEQ ID NO:51, which comprises the sequence X₁GX₃X₄X₆X₆X₇AGGTAAGTG (SEQ ID NO:338), wherein X of any of X₁ and X₃-X₇ can be A, C, T or G, in any combination;

s2) the nucleotide sequence of SEQ ID NO:52, in any combination, which comprises the sequence X₁X₂GX₄X₆X₆X₇AGGTAAGTG (SEQ ID NO:339), wherein X of any of X₁-X₂ and X₄-X₇ can be A, C, T or G;

t2) the nucleotide sequence of SEQ ID NO:53, which comprises the sequence X₁X₂GX₄X₆X₆X₇AGGTAAGTG (SEQ ID NO:339), wherein X of any of X₁-X₂ and X₄-X₇ can be A, C, T or G, in any combination;

u2) the nucleotide sequence of SEQ ID NO:54, which comprises the sequence X₁X₂X₃GX₅X₈X₇AGGTAAGTG (SEQ ID NO:340), wherein X of any of X₁-X₃ and X₅-X₇ can be A, C, T or G, in any combination;

v2) the nucleotide sequence of SEQ ID NO:55, which comprises the sequence X₁X₂X₃GX₅X₈X₇AGGTAAGTG (SEQ ID NO:340), wherein X of any of X₁-X₃ and X₅-X₇ can be A, C, T or G, in any combination;

w2) the nucleotide sequence of SEQ ID NO:56, which comprises the sequence X₁X₂X₃X₄TX₈X₇AGGTAAGTG (SEQ ID NO:341), wherein X of any of X₁-X₄ and X₆-X₇ can be A, C, T or G, in any combination;

x2) the nucleotide sequence of SEQ ID NO:57, which comprises the sequence X₁X₂X₃X₄TX₈X₇AGGTAAGTG (SEQ ID NO:341), wherein X of any of X₁-X₄ and X₆-X₇ can be A, C, T or G, in any combination;

y2) the nucleotide sequence of SEQ ID NO:58, which comprises the sequence X₁GGX₄X₅X₆X₇X₈GGTAAGTG (SEQ ID NO:342), wherein X of any of X₁ and X₄-X₅ can be A, C, T or G, in any combination;

z2) the nucleotide sequence of SEQ ID NO:59, which comprises the sequence X₁GGX₄X₅X₆X₇X₈GGTAAGTG (SEQ ID NO:342), wherein X of any of X₁ and X₄-X₈ can be A, C, T or G, in any combination;

a3) the nucleotide sequence of SEQ ID NO:60, which comprises the sequence X₁X₂GGX₅X₈X₇X₈GGTAAGTG (SEQ ID NO:343), wherein X of any of X₁-X₂ and X₅-X₈ can be A, C, T or G, in any combination;

b3) the nucleotide sequence of SEQ ID NO:61, which comprises the sequence X₁X₂GGX₅X₈X₇X₈GGTAAGTG (SEQ ID NO:343), wherein X of any of X₁-X₂ and X₅-X₈ can be A, C, T or G, in any combination;

c3) the nucleotide sequence of SEQ ID NO:62, which comprises the sequence X₁X₂X₃GTX₈X₇X₈GGTAAGTG (SEQ ID NO:344), wherein X of any of X₁-X₂ and X₅-X₈ can be A, C, T or G, in any combination;

d3) the nucleotide sequence of SEQ ID NO:63, which comprises the sequence X₁X₂X₃GTX₈X₇X₈GGTAAGTG (SEQ ID NO:344), wherein X of any of X₁-X₃ and X₆-X₈ can be A, C, T or G, in any combination;

e3) the nucleotide sequence of SEQ ID NO:64, which comprises the sequence X₁X₂X₃X₄TTX₇X₈GGTAAGTG (SEQ ID NO:345), wherein X of any of X₁-X₄ and X₇-X₈ can be A, C, T or G, in any combination;

f3) the nucleotide sequence of SEQ ID NO:65, which comprises the sequence X₁X₂X₃X₄TTX₇X₈GGTAAGTG (SEQ ID NO:345), wherein X of any of X₁-X₄ and X₇-X₈ can be A, C, T or G, in any combination;

g3) the nucleotide sequence of SEQ ID NO:66, which comprises the sequence X₁X₂X₃X₄X₅TAX₈GGTAATGTG (SEQ ID NO:346), wherein X of any of X₁-X₅ and X₈ can be A, C, T or G, in any combination;

h3) the nucleotide sequence of SEQ ID NO:67, which comprises the sequence X₁X₂X₃X₄X₅TAX₈GGTAATGTG (SEQ ID NO:346), wherein X of any of X₁-X₅ and X₈ can be A, C, T or G, in any combination;

i3) the nucleotide sequence of SEQ ID NO:68, which comprises the sequence AGCGAATAGGTAATAC (SEQ ID NO:347);

j3) the nucleotide sequence of SEQ ID NO:69, which comprises the sequence to AGCGAATAGGTAATAC (SEQ ID NO:347);.

k3) the nucleotide sequence of SEQ ID NO:70, which comprises the sequence CGAGGGCAGGTAATAA (SEQ ID NO:348);

l3) the nucleotide sequence of SEQ ID NO:71, which comprises the sequence CGAGGGCAGGTAATAA (SEQ ID NO:348);

m3) the nucleotide sequence of SEQ ID NO:72, which comprises the sequence GGTGCCGAGGTAATAT (SEQ ID NO:349);

n3) the nucleotide sequence of SEQ ID NO:73, which comprises the sequence GGTGCCGAGGTAATAT (SEQ ID NO:349);

o3) the nucleotide sequence of SEQ ID NO:74, which comprises the sequence GGTGACTAGGTAATAC (SEQ ID NO:350);

p3) the nucleotide sequence of SEQ ID NO:75, which comprises the sequence GGTGACTAGGTAATAC (SEQ ID NO:350);

q3) the nucleotide sequence of SEQ ID NO:76, which comprises the sequence CGAGGGCAGGTAATAT (SEQ ID NO:351);

r3) the nucleotide sequence of SEQ ID NO:77, which comprises the sequence CGAGGGCAGGTAATAT (SEQ ID NO:351);

s3) the nucleotide sequence of SEQ ID NO:78, which comprises the sequence AGCGCAGAGGTAATAA (SEQ ID NO:352);

t3) the nucleotide sequence of SEQ ID NO:79, which comprises the sequence AGCGCAGAGGTAATAA (SEQ ID NO:352);

u3) the nucleotide sequence of SEQ ID NO:80, which comprises the sequence AGCGAATAGGTAAGTC (SEQ ID NO:353);

v3) the nucleotide sequence of SEQ ID NO:81, which comprises the sequence AGCGAATAGGTAAGTC (SEQ ID NO:353);

w3) the nucleotide sequence of SEQ ID NO:82, which comprises the sequence CGAGGGCAGGTAAGTA (SEQ ID NO:354);

x3) the nucleotide sequence of SEQ ID NO:83, which comprises the sequence CGAGGGCAGGTAAGTA (SEQ ID NO:354);

y3) the nucleotide sequence of SEQ ID NO:84, which comprises the sequence GGTGCCGAGGTAAGTT (SEQ ID NO:355);

z3) the nucleotide sequence of SEQ ID NO:85, which comprises the sequence GGTGCCGAGGTAAGTT (SEQ ID NO:355);

a4) the nucleotide sequence of SEQ ID NO:86, which comprises the sequence GGTGACTAGGTAAGTC (SEQ ID NO:356);

b4) the nucleotide sequence of SEQ ID NO:87, which comprises the sequence GGTGACTAGGTAAGTC (SEQ ID NO:356);

c4) the nucleotide sequence of SEQ ID NO:88, which comprises the sequence CGAGGGCAGGTAAGTT (SEQ ID NO:357);

d4) the nucleotide sequence of SEQ ID NO:89, which comprises the sequence CGAGGGCAGGTAAGTT (SEQ ID NO:357);

e4) the nucleotide sequence of SEQ ID NO:90, which comprises the sequence AGCGCAGAGGTAAGTA (SEQ ID NO:358);

f4) the nucleotide sequence of SEQ ID NO:91, which comprises the sequence AGCGCAGAGGTAAGTA (SEQ ID NO:358);

g4) the nucleotide sequence of SEQ ID NO:10;

h4) the nucleotide sequence of SEQ ID NO:11;

i4) the nucleotide sequence of SEQ ID NO:12;

j4) the nucleotide sequence of SEQ ID NO:13;

k4) the nucleotide sequence of SEQ ID NO:18;

l4) the nucleotide sequence of SEQ ID NO:20;

m4) the nucleotide sequence of SEQ ID NO:19;

n4) the nucleotide sequence of SEQ ID NO:21; and

o4) any combination of a1 through n4 above.

As further embodiments, in the cell of this invention, a second intronic nucleotide sequence of either or both of the first and second heterologous nucleic acid constructs can comprise, in any combination, the nucleotide sequence of any of SEQ ID NOs:243-278, 292-316, 320 and 321, either as set forth as any of SEQ ID NOs:243-278, 292-316, 320 and 321, respectively, and/or as a modification of a nucleotide sequence of any of SEQ ID NOs:243-278, 292-316, 320 and 321, wherein the nucleotide sequence is modified at the appropriate site(s) as would be recognized by one of ordinary skill in the art, to incorporate the mutant intron sequences, in any combination, as set forth in SEQ ID NOS:1-242.

Any of these nucleotide sequences can be present in either one of the first and second heterologous nucleic acid constructs or in both of the first and second heterologous nucleic acid constructs and the nucleotide sequences can be present in either or both of the first and second nucleic acid constructs singly and/or in multiples in any combination relative to one another and relative to the first and second heterologous nucleic acid constructs. Furthermore, in embodiments in which these nucleotide sequences are present in both first and second heterologous nucleic acid constructs, they can all be the same, some can be the same and some can be different and/or they can all be different.

Furthermore, in the cell of this invention, the first heterologous nucleic acid construct and/or the second heterologous nucleic acid construct can comprise two or more (e.g., 2, 3, 4, 5, 6, 7, 8, 9, 10, etc.) second intronic nucleotide sequences.

In additional embodiments, in the cell of this invention, the first heterologous nucleic acid construct and/or the second heterologous nucleic acid construct can comprise two or more second intronic nucleotide sequences that can be, but are not limited to, a) second intronic nucleotide sequences in tandem within said first nucleotide sequence, b) second intronic nucleotide sequences spaced at least 25 base pairs apart within said first nucleotide sequence, c) second intronic nucleotide sequences spaced at least 50 base pairs apart within said first nucleotide sequence, d) second intronic nucleotide sequences spaced at least 75 base pairs apart within said first nucleotide sequence, e) second intronic nucleotide, sequences spaced at least 100 base pairs apart within said first nucleotide sequence, f) second intronic nucleotide sequences spaced at least 200 base pairs apart within said first nucleotide sequence, g) second intronic nucleotide sequences spaced at least 300 base pairs apart within said first nucleotide sequence, h) second intronic nucleotide sequences wherein a primary second intronic nucleotide sequence is located between a promoter and said heterologous first nucleotide sequence and a secondary second intronic nucleotide sequence is located within said first nucleotide sequence; and i) second intronic nucleotide sequences wherein a primary second intronic nucleotide sequence is located between an open reading frame and a poly A signal in said first nucleotide sequence and a secondary intronic second nucleotide sequence is located within said open reading frame of said first nucleotide sequence.

In those embodiments in which two or more second intronic nucleotide sequences are present in the first and/or second heterologous nucleic acid construct, the two or more second intronic nucleotide sequences can all be the same, can all be different and/or can be any combination of same and different nucleotide sequences.

The present invention additionally provides embodiments of the cell of this invention wherein the first heterologous nucleic acid construct and/or the second heterologous nucleic acid construct comprises two or more (e.g., 2, 3, 4, 5, 6, 7, 8, 9, 10, etc.) first nucleotide sequences.

In those embodiments in which two or more first nucleotide sequences are present in the first and/or second heterologous nucleic acid construct, the two or more first nucleotide sequences can all be the same, can all be different and/or can be any combination of same and different nucleotide sequences.

In various embodiments, the first heterologous nucleic acid construct and/or the second heterologous nucleic acid construct can further comprise a promoter that directs expression of the first nucleotide sequence(s) of the first heterologous nucleic acid construct and/or the second heterologous nucleic acid construct.

In such embodiments, the second intronic nucleotide sequence of the first heterologous nucleic acid construct and/or of the second heterologous nucleic acid construct can be positioned between the promoter and the first nucleotide sequence(s) of the first heterologous nucleic acid construct and/or the second heterologous nucleic acid construct.

In other embodiments of the cell of this invention, the second intronic nucleotide sequence of the first heterologous nucleic acid construct and/or the second heterologous nucleic acid construct can be positioned within an open reading frame of the first nucleotide sequence(s) of the first heterologous nucleic acid construct and/or the second heterologous nucleic acid construct.

In yet other embodiments, the second intronic nucleotide sequence of the first heterologous nucleic acid construct and/or the second heterologous nucleic acid construct can be positioned 1) within the 5′ one-third of an open reading frame of said first nucleotide sequence(s) of the first heterologous nucleic acid construct and/or the second heterologous nucleic acid construct, 2) within the middle one-third of an open reading frame of the first nucleotide sequence(s) of the first heterologous nucleic acid construct and/or the second heterologous nucleic acid construct, 3) within the 3′ one-third of an open reading frame of the first nucleotide sequence(s) of the first heterologous nucleic acid construct and/or the second heterologous nucleic acid construct, and/or 4) between an open reading frame and a poly A signal in the first nucleotide sequence(s) of the first heterologous nucleic acid construct and/or the second heterologous nucleic acid construct, including any combination of (1)-(4) above relative to one another and relative to the first heterologous nucleic acid construct and the second heterologous nucleic acid construct.

The phrase “a first RNA molecule corresponding to said first nucleotide sequence” describes an RNA molecule that is encoded by and thus produced by transcription of the first nucleotide sequence present in the heterologous nucleic acid construct under conditions in which the second set of splice elements is inactive, i.e., the second set of splice elements is rendered inactive due to the presence of a blocking oligonucleotide, small molecule or other compound as described herein. When splicing occurs under the direction of the first set of splice elements, which are the elements that define the first intron (e.g., the “correct” splice sites), to remove the first intron present in the second intronic nucleotide sequence, the result is the production of a first RNA molecule corresponding to (i.e., encoded by) said first nucleotide sequence, which is the RNA molecule that is either translated into a functional peptide or protein or is it itself a functional RNA molecule as described herein The phrase “no RNA molecule or a second RNA molecule that is not encoded by said first nucleotide sequence” describes the result when splicing activity under the direction of the second set of splice elements, which define aberrant or alternative splice sites that are different from the “correct” splice sites, the splicing activity at which yields the functional RNA encoded by the first nucleotide sequence. “No RNA molecule or a second RNA molecule that is not encoded by said first nucleotide sequence” is the intended result when production of a functional protein, peptide and/or RNA encoded by the first nucleotide sequence is not desired, i.e., when the transgene expression system is in the OFF position. In some embodiments, splicing under the direction of the second (i.e., aberrant or alternate) set of splice elements can result in no RNA production at all and/or an RNA that is not capable of being translated into a functional protein or peptide and/or is not capable of activity as a functional RNA as encoded by the first nucleotide sequence. In further embodiments, splicing at the second set of splice elements can result in the production of a second functional RNA that is different than the first functional RNA encoded by the first nucleotide sequence. Thus, in some embodiments, splicing at the first set of splice elements to remove the first intron results in production of a first functional RNA encoded by the first nucleotide sequence, while in the same nucleic acid construct, splicing at the second set of splice elements to remove the second intron results in the production of a second functional RNA that is different from said first functional RNA.

The present invention further provides mutant introns that can be employed to differentially regulate the expression of one or more transgenes in a cell (e.g., simultaneously, sequentially, etc., in any combination). Thus, provided herein is an isolated nucleic acid comprising, consisting essentially of or consisting of: A) a first nucleotide sequence encoding a nucleotide sequence of interest (NOI); and B) a second intronic nucleotide sequence selected from the group consisting of:

a1) the nucleotide sequence of SEQ ID NO:92;

b1) the nucleotide sequence of SEQ ID NO:2;

c1) the nucleotide sequence of SEQ ID NO:1;

d1) the nucleotide sequence of SEQ ID NO:4;

e1) the nucleotide sequence of SEQ ID NO:3;

f1) the nucleotide sequence of SEQ ID NO:5;

g1) the nucleotide sequence of SEQ ID NO:6;

h1) the nucleotide sequence of SEQ ID NO:7;

i1) the nucleotide sequence of SEQ ID NO:8;

j1) the nucleotide sequence of SEQ ID NO:9;

k1) the nucleotide sequence of SEQ ID NO:14;

l1) the nucleotide sequence of SEQ ID NO:16;

m1) the nucleotide sequence of SEQ ID NO:15;

n1) the nucleotide sequence of SEQ ID NO:17;

o1) the nucleotide sequence of SEQ ID NO:22, which comprises the sequence X₁X₂X₃X₄X₆X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

p1) the nucleotide sequence of SEQ ID NO:23, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;

q1) the nucleotide sequence of SEQ ID NO:24, which comprises the sequence X₁GX₃GX₆X₆X₇AGGTAATAX₁₄ (SEQ ID NO:325), wherein X of any of X₁, X₃-X₇ and X₁₄ can be A, C, T or G, in any combination;

r1) the nucleotide sequence of SEQ ID NO:25, which comprises the sequence X₁GX₃GX₆X₆X₇AGGTAATAX₁₄ (SEQ ID NO:325), wherein X of any of X₁, X₃-X₇ and X₁₄ can be A, C, T or G, in any combination;

s1) the nucleotide sequence of SEQ ID NO:26, which comprises the sequence X₁GX₃GX₆X₆X₇AGGTAAGTX₁₄ (SEQ ID NO:326), wherein X of any of X₁, X₃, X₅-X₇ and X₁₄ can be A, C, T or G, in any combination;

t1) the nucleotide sequence of SEQ ID NO:27, which comprises the sequence X₁GX₃GX₆X₆X₇AGGTAAGTX₁₄ (SEQ ID NO:326), wherein X of any of X₁, X₃, X₅-X₇ and X₁₄ can be A, C, T or G, in any combination;

u1) the nucleotide sequence of SEQ ID NO:28, which comprises the sequence X₁X₂X₃X₄X₆X₆AAGGTAATAG (SEQ ID NO:327), wherein X of any of X₁,-X₆ can be A, C, T or G, in any combination;

v1) the nucleotide sequence of SEQ ID NO:29, which comprises the sequence X₁X₂X₃X₄X₆X₆AAGGTAATAG (SEQ ID NO:327), wherein X of any of X₁,-X₆ can be A, C, T or G, in any combination;

w1) the nucleotide sequence of SEQ ID NO:30, which comprises the sequence X₁GX₃X₄X₆X₆X₇AGGTAATAG (SEQ ID NO:328), wherein X of any of X₁ and X₃-X₇ can be A, C, T or G, in any combination;

x1) the nucleotide sequence of SEQ ID NO:31, which comprises the sequence X₁GX₃X₄X₆X₆X₇AGGTAATAG (SEQ ID NO:328), wherein X of any of X₁, and X₃-X₇ can be A, C, T or G, in any combination;

y1) the nucleotide sequence of SEQ ID NO:32, which comprises the sequence X₁X₂GX₄X₅X₆X₇AGGTAATAG (SEQ ID NO:329), wherein X of any of X₁, X₂ and X₄-X₇ can be A, C, T or G, in any combination;

z1) the nucleotide sequence of SEQ ID NO:33, which comprises the sequence X₁X₂GX₄X₅X₈X₇AGGTAATAG (SEQ ID NO:329), wherein X of any of X₁, X₂ and X₄-X₇ can be A, C, T or G, in any combination;

a2) the nucleotide sequence of SEQ ID NO:34, which comprises the sequence X₁X₂X₃GX₅X₈X₇AGGTAATAG (SEQ ID NO:330), wherein X of any of X₁-X₃ and X₅-X₇ can be A, C, T or G, in any combination;

b2) the nucleotide sequence of SEQ ID NO:35, which comprises the sequence X₁X₂X₃GX₅X₈X₇AGGTAATAG (SEQ ID NO:330), wherein X of any of X₁-X₃ and X₅-X₇ can be A, C, T or G, in any combination;

c2) the nucleotide sequence of SEQ ID NO:36, which comprises the sequence X₁X₂X₃X₄TX₆X₇AGGTAATAG (SEQ ID NO:331), wherein X of any of X₁-X₄ and X₆-X₇ can be A, C, T or G, in any combination;

d2) the nucleotide sequence of SEQ ID NO:37, which comprises the sequence X₁X₂X₃X₄TX₈X₇AGGTAATAG (SEQ ID NO:331), wherein X of any of X₁-X₄ and X₆-X₇ can be A, C, T or G, in any combination;

e2) the nucleotide sequence of SEQ ID NO:38, which comprises the sequence X₁GGX₄X₅X₈X₇X₈GGTAATAG (SEQ ID NO:332), wherein X of any of X₁ and X₄-X₈ can be A, C, T or G, in any combination;

f2) the nucleotide sequence of SEQ ID NO:39, which comprises the sequence X₁GGX₄X₅X₈X₇X₈GGTAATAG (SEQ ID NO:332), wherein X of any of X₁ and X₄-X₈ can be A, C, T or G, in any combination;

g2) the nucleotide sequence of SEQ ID NO:40, which comprises the sequence X₁X₂GGX₅X₈X₇X₈GGTAATAG (SEQ ID NO:333), wherein X of any of X₁, X₂ and X₅-X₈ can be A, C, T or G, in any combination;

h2) the nucleotide sequence of SEQ ID NO:41, which comprises the sequence X₁X₂GGX₅X₈X₇X₈GGTAATAG (SEQ ID NO:333), wherein X of any of X₁, X₂ and X₅-X₈ can be A, C, T or G, in any combination;

i2) the nucleotide sequence of SEQ ID NO:42, which comprises the sequence X₁X₂X₃GTX₈X₇X₈GGTAATAG (SEQ ID NO:334), wherein X of any of X₁-X₃ and X₆-X₈ can be A, C, T or G, in any combination;

j2) the nucleotide sequence of SEQ ID NO:43, which comprises the sequence X₁X₂X₃GTX₈X₇X₈GGTAATAG (SEQ ID NO:334), wherein X of any of X₁-X₃ and X₆-X₈ can be A, C, T or G, in any combination;

k2) the nucleotide sequence of SEQ ID NO:44, which comprises the sequence X₁X₂X₃X₄TTX₇X₈GGTAATAG (SEQ ID NO:335), wherein X of any of X₁-X₄ and X₇-X₈ can be A, C, T or G, in any combination;

l2) the nucleotide sequence of SEQ ID NO:45, which comprises the sequence X₁X₂X₃X₄TTX₇X₈GGTAATAG (SEQ ID NO:335), wherein X of any of X₁-X₄ and X₇-X₈ can be A, C, T or G, in any combination;

m2) the nucleotide sequence of SEQ ID NO:46, which comprises the sequence X₁X₂X₃X₄X₈TAX₈GGTAATAG (SEQ ID NO:336), wherein X of any of X₁-X₅ and X₈ can be A, C, T or G, in any combination;

n2) the nucleotide sequence of SEQ ID NO:47, which comprises the sequence X₁X₂X₃X₄X₈TAX₈GGTAATAG (SEQ ID NO:336), wherein X of any of X₁-X₅ and X₈ can be A, C, T or G, in any combination;

o2) the nucleotide sequence of SEQ ID NO:48, which comprises the sequence X₁X₂X₃X₄X₈X₈AAGGTAAGTG (SEQ ID NO:337), wherein X of any of X₁-X₈ can be A, C, T or G, in any combination;

p2) the nucleotide sequence of SEQ ID NO:49, which comprises the sequence X₁X₂X₃X₄X₈X₈AAGGTAAGTG (SEQ ID NO:337), wherein X of any of X₁-X₈ can be A, C, T or G, in any combination;

q2) the nucleotide sequence of SEQ ID NO:50, which comprises the sequence X₁GX₃X₄X₈X₈X₇AGGTAAGTG (SEQ ID NO:338), wherein X of any of X₁ and X₃-X₇ can be A, C, T or G, in any combination;

r2) the nucleotide sequence of SEQ ID NO:51, which comprises the sequence X₁GX₃X₄X₈X₈X₇AGGTAAGTG (SEQ ID NO:338), wherein X of any of X₁ and X₃-X₇ can be A, C, T or G, in any combination;

s2) the nucleotide sequence of SEQ ID NO:52, in any combination, which comprises the sequence X₁X₂GX₄X₈X₈X₇AGGTAAGTG (SEQ ID NO:339), wherein X of any of X₁-X₂ and X₄-X₇ can be A, C, T or G;

t2) the nucleotide sequence of SEQ ID NO:53, which comprises the sequence X₁X₂GX₄X₈X₈X₇AGGTAAGTG (SEQ ID NO:339), wherein X of any of X₁-X₂ and X₄-X₇ can be A, C, T or G, in any combination;

u2) the nucleotide sequence of SEQ ID NO:54, which comprises the sequence X₁X₂X₃GX₈X₈X₇AGGTAAGTG (SEQ ID NO:340), wherein X of any of X₁-X₃ and X₅-X₇ can be A, C, T or G, in any combination;

v2) the nucleotide sequence of SEQ ID NO:55, which comprises the sequence X₁X₂X₃GX₈X₈X₇AGGTAAGTG (SEQ ID NO:340), wherein X of any of X₁-X₃ and X₅-X₇ can be A, C, T or G, in any combination;

w2) the nucleotide sequence of SEQ ID NO:56, which comprises the sequence X₁X₂X₃X₄TX₆X₇AGGTAAGTG (SEQ ID NO:341), wherein X of any of X₁-X₄ and X₆-X₇ can be A, C, T or G, in any combination;

x2) the nucleotide sequence of SEQ ID NO:57, which comprises the sequence X₁X₂X₃X₄TX₆X₇AGGTAAGTG (SEQ ID NO:341), wherein X of any of X₁-X₄ and X₆-X₇ can be A, C, T or G, in any combination;

y2) the nucleotide sequence of SEQ ID NO:58, which comprises the sequence X₁GGX₄X₅X₆X₇X₈GGTAAGTG (SEQ ID NO:342), wherein X of any of X₁ and X₄-X₈ can be A, C, T or G, in any combination;

z2) the nucleotide sequence of SEQ ID NO:59, which comprises the sequence X₁GGX₄X₅X₆X₇X₈GGTAAGTG (SEQ ID NO:342), wherein X of any of X₁ and X₄-X₈ can be A, C, T or G, in any combination;

a3) the nucleotide sequence of SEQ ID NO:60, which comprises the sequence X₁X₂GGX₅X₆X₇X₈GGTAAGTG (SEQ ID NO:343), wherein X of any of X₁-X₂ and X₅-X₈ can be A, C, T or G, in any combination;

b3) the nucleotide sequence of SEQ ID NO:61, which comprises the sequence X₁X₂GGX₅X₆X₇X₈GGTAAGTG (SEQ ID NO:343), wherein X of any of X₁-X₂ and X₅-X₈ can be A, C, T or G, in any combination;

c3) the nucleotide sequence of SEQ ID NO:62, which comprises the sequence X₁X₂X₃GTX₆X₇X₈GGTAAGTG (SEQ ID NO:344), wherein X of any of X₁-X₂ and X₅-X₈ can be A, C, T or G, in any combination;

d3) the nucleotide sequence of SEQ ID NO:63, which comprises the sequence X₁X₂X₃GTX₆X₇X₈GGTAAGTG (SEQ ID NO:344), wherein X of any of X₁-X₃ and X₆-X₈ can be A, C, T or G, in any combination;

e3) the nucleotide sequence of SEQ ID NO:64, which comprises the sequence X₁X₂X₃X₄TTX₇X₈GGTAAGTG (SEQ ID NO:345), wherein X of any of X₁-X₄ and X₇-X₈ can be A, C, T or G, in any combination;

f3) the nucleotide sequence of SEQ ID NO:65, which comprises the sequence X₁X₂X₃X₄TTX₇X₈GGTAAGTG (SEQ ID NO:345), wherein X of any of X₁-X₄ and X₇-X₈ can be A, C, T or G, in any combination;

g3) the nucleotide sequence of SEQ ID NO:66, which comprises the sequence X₁X₂X₃X₄X₅TAX₈GGTAATGTG (SEQ ID NO:346), wherein X of any of X₁-X₅ and X₈ can be A, C, T or G, in any combination;

h3) the nucleotide sequence of SEQ ID NO:67, which comprises the sequence X₁X₂X₃X₄X₅TAX₈GGTAATGTG (SEQ ID NO:346), wherein X of any of X₁-X₅ and X₈ can be A, C, T or G, in any combination;

i3) the nucleotide sequence of SEQ ID NO:68, which comprises the sequence AGCGAATAGGTAATAC (SEQ ID NO:347);

j3) the nucleotide sequence of SEQ ID NO:69, which comprises the sequence AGCGAATAGGTAATAC (SEQ ID NO:347);

k3) the nucleotide sequence of SEQ ID NO:70, which comprises the sequence CGAGGGCAGGTAATAA (SEQ ID NO:348);

l3) the nucleotide sequence of SEQ ID NO:71, which comprises the sequence CGAGGGCAGGTAATAA (SEQ ID NO:348);

m3) the nucleotide sequence of SEQ ID NO:72, which comprises the sequence GGTGCCGAGGTAATAT (SEQ ID NO:349);

n3) the nucleotide sequence of SEQ ID NO:73, which comprises the sequence GGTGCCGAGGTAATAT (SEQ ID NO:349);

o3) the nucleotide sequence of SEQ ID NO:74, which comprises the sequence GGTGACTAGGTAATAC (SEQ ID NO:350);

p3) the nucleotide sequence of SEQ ID NO:75, which comprises the sequence GGTGACTAGGTAATAC (SEQ ID NO:350);

q3) the nucleotide sequence of SEQ ID NO:76, which comprises the sequence CGAGGGCAGGTAATAT (SEQ ID NO:351);

r3) the nucleotide sequence of SEQ ID NO:77, which comprises the sequence CGAGGGCAGGTAATAT (SEQ ID NO:351);

s3) the nucleotide sequence of SEQ ID NO:78, which comprises the sequence AGCGCAGAGGTAATAA (SEQ ID NO:352);

t3) the nucleotide sequence of SEQ ID NO:79, which comprises the sequence AGCGCAGAGGTAATAA (SEQ ID NO:352);

u3) the nucleotide sequence of SEQ ID NO:80, which comprises the sequence AGCGAATAGGTAAGTC (SEQ ID NO:353);

v3) the nucleotide sequence of SEQ ID NO:81, which comprises the sequence AGCGAATAGGTAAGTC (SEQ ID NO:353);

w3) the nucleotide sequence of SEQ ID NO:82, which comprises the sequence CGAGGGCAGGTAAGTA (SEQ ID NO:354);

x3) the nucleotide sequence of SEQ ID NO:83, which comprises the sequence CGAGGGCAGGTAAGTA (SEQ ID NO:354);

y3) the nucleotide sequence of SEQ ID NO:84, which comprises the sequence GGTGCCGAGGTAAGTT (SEQ ID NO:355);

z3) the nucleotide sequence of SEQ ID NO:85, which comprises the sequence GGTGCCGAGGTAAGTT (SEQ ID NO:355);

a4) the nucleotide sequence of SEQ ID NO:86, which comprises the sequence GGTGACTAGGTAAGTC (SEQ ID NO:356);

b4) the nucleotide sequence of SEQ ID NO:87, which comprises the sequence GGTGACTAGGTAAGTC (SEQ ID NO:356);

c4) the nucleotide sequence of SEQ ID NO:88, which comprises the sequence CGAGGGCAGGTAAGTT (SEQ ID NO:357);

d4) the nucleotide sequence of SEQ ID NO:89, which comprises the sequence CGAGGGCAGGTAAGTT (SEQ ID NO:357);

e4) the nucleotide sequence of SEQ ID NO:90, which comprises the sequence AGCGCAGAGGTAAGTA (SEQ ID NO:358);

f4) the nucleotide sequence of SEQ ID NO:91, which comprises the sequence AGCGCAGAGGTAAGTA (SEQ ID NO:358);

g4) the nucleotide sequence of SEQ ID NO:10;

h4) the nucleotide sequence of SEQ ID NO:11;

i4) the nucleotide sequence of SEQ ID NO:12;

j4) the nucleotide sequence of SEQ ID NO:13;

k4) the nucleotide sequence of SEQ ID NO:18;

l4) the nucleotide sequence of SEQ ID NO:20;

m4) the nucleotide sequence of SEQ ID NO:19;

n4) the nucleotide sequence of SEQ ID NO:21; and

o4) any combination of a1 through n4 above.

As further embodiments, in the isolated nucleic acid of this invention, the second intronic nucleotide sequence can comprise, in any combination, the nucleotide sequence of any of SEQ ID NOs:243-278, 292-316, 320 and 321 with a modification wherein the nucleotide sequence of SEQ ID NOs:243-278, 292-316, 320 and 321 is modified at the appropriate site(s) as would be recognized by one of ordinary skill in the art, to incorporate, in any combination, the mutant intron sequences as set forth in SEQ ID NOS:1-242.

Any of these nucleotide sequences can be present singly and/or in multiples in any combination. Furthermore, the isolated nucleic acid of this invention can comprises two or more (e.g., 2, 3, 4, 5, 6, 7, 8, 9, 10, etc.) second intronic nucleotide sequences, which can all be the same, all be different or any combination of some being the same and some being different.

In additional embodiments, the isolated nucleic acid molecule of this invention can comprise two or more (e.g., 2, 3, 4, 5, 6, 7, 8, 9, 10, etc.) second intronic nucleotide sequences that can be, but are not limited to, a) second intronic nucleotide sequences in tandem within said first nucleotide sequence, b) second intronic nucleotide sequences spaced at least 25 base pairs apart within said first nucleotide sequence, c) second intronic nucleotide sequences spaced at least 50 base pairs apart within said first nucleotide sequence, d) second intronic nucleotide sequences spaced at least 75 base pairs apart within said first nucleotide sequence, e) second intronic nucleotide sequences spaced at least 100 base pairs apart within said first nucleotide sequence, f) second intronic nucleotide sequences spaced at least 200 base pairs apart within said first nucleotide sequence, g) second intronic nucleotide sequences spaced at least 300 base pairs apart within said first nucleotide sequence, h) second intronic nucleotide sequences wherein a primary second intronic nucleotide sequence is located between a promoter and said heterologous first nucleotide sequence and a secondary second intronic nucleotide sequence is located within to said first nucleotide' sequence; and i) second intronic nucleotide sequences wherein a primary second intronic nucleotide sequence is located between an open reading frame and a poly A signal (e.g., poly A nucleotide sequence) in said first nucleotide sequence and a secondary heterologous second nucleotide sequence located within said open reading frame of said first nucleotide sequence.

In those embodiments in which two or more second intronic nucleotide sequences are present in the isolated nucleic acid molecule of this invention, the two or more second intronic nucleotide sequences can all be the same, can all be different and/or can be any combination of same and different nucleotide sequences.

The present invention additionally provides embodiments wherein the isolated nucleic acid of this invention comprises two or more (e.g., 2, 3, 4, 5, 6, 7, 8, 9, 10, etc.) first nucleotide sequences.

In those embodiments in which two or more first nucleotide sequences are present in the isolated nucleic acid molecule, the two or more first nucleotide sequences can all be the same, can all be different and/or can be any combination of same and different nucleotide sequences.

In various embodiments, the isolated nucleic acid molecule can further comprise a promoter that directs expression of the first nucleotide sequence(s). In such embodiments, the second intronic nucleotide sequence can be positioned between the promoter and the first nucleotide sequence(s).

In other embodiments of the isolated nucleic acid of this invention, the second intronic nucleotide sequence can be positioned within an open reading frame of the first nucleotide sequence(s).

In yet other embodiments, the second intronic nucleotide sequence can be positioned 1) within the 5′ one-third of an open reading frame of said first nucleotide sequence(s), 2) within the middle one-third of an open reading frame of the first nucleotide sequence(s), 3) within the 3′ one-third of an open reading frame of the first nucleotide sequence(s), and/or 4) between an open reading frame and a poly A signal (i.e., sequence) in the first nucleotide sequence(s), including any combination of (1)-(4) above.

Additionally provided herein is a vector comprising an isolated nucleic acid molecule of this invention, as well as a cell comprising a nucleic acid of this invention and a cell comprising a vector of this invention.

The present invention further includes compositions, including a composition comprising an isolated nucleic acid molecule of this invention in a pharmaceutically acceptable carrier. Also provided is a composition comprising a cell of this invention in a pharmaceutically acceptable carrier, as well as a composition comprising a vector of this invention in a pharmaceutically acceptable carrier.

It is further intended that the cells, isolated nucleic acids, vectors and compositions of this invention be employed in methods to control and differentially regulate the expression of a transgene (e.g., NOI) or multiple transgenes (e.g., NOIs). Such transgenes may be present in a cell that is introduced into a subject to deliver or introduce the transgene into the subject and/or to impart to the subject a treatment or therapeutic effect. Such transgenes may also be present as an isolated nucleic acid or in a nucleic acid vector that is introduced into a subject to deliver or introduce the transgene into cells of the subject and/or to impart to the subject a treatment or therapeutic effect.

As used herein, the terms “transgene,” “gene” and “coding sequence” are used interchangeably to describe nucleotide sequence of interest (NOI) present in the first heterologous nucleotide of this invention. In some embodiments, the NOI can be a coding sequence (i.e., without introns and other regulatory elements that would be present in the genomic version of the NOI) and in other embodiments can be a genomic sequence (i.e., defined by exons, introns and other regulatory elements present in the genomic version of the NOI). Thus, it is understood that the second intronic nucleotide sequence provides an intron sequence that is not associated in its natural state with the first nucleotide sequence with which the second intronic nucleotide sequence is operably associated in the heterologous nucleic acid constructs and isolated nucleic acids of this invention.

Thus, in one embodiment, the present invention provides a method of producing a functional product (e.g., RNA) encoded by said first nucleotide sequence of said first heterologous nucleic acid construct or said second heterologous nucleic acid construct in said cell of this invention, comprising: introducing into said cell a blocking oligonucleotide and/or small molecule that blocks a member of said second set of splice elements of said second intronic nucleotide sequence of said first heterologous nucleic acid construct or a blocking oligonucleotide and/or small molecule that blocks a member of said second set of splice elements of said second intronic nucleotide sequence of said second heterologous nucleic acid construct, thereby producing the functional product (e.g., RNA) encoded by the first nucleotide sequence of said first heterologous nucleic acid construct or said second heterologous nucleic acid construct in said cell.

Further provided herein is a method of producing a first functional product encoded by said first nucleotide sequence of said first heterologous nucleic acid construct and producing a second functional product encoded by said first nucleotide sequence of said second heterologous nucleic acid construct in said cell of this invention, wherein said first functional product and said second functional product are different from each other, comprising: a) introducing into said cell a first blocking oligonucleotide and/or first small molecule that blocks a member of said second set of splice elements of said second intronic nucleotide sequence of said first heterologous nucleic acid construct, thereby producing said first functional product encoded by said first nucleotide sequence of said first heterologous nucleic acid construct in said cell, and b) introducing into said cell a second blocking oligonucleotide and/or second small molecule that blocks a member of said second set of splice elements of said second intronic nucleotide sequence of said second heterologous nucleic acid construct, thereby producing said second functional product encoded by the first nucleotide sequence of said second heterologous nucleic acid construct in said cell, wherein said second intronic nucleotide sequence of said first heterologous nucleic acid construct and said second intronic nucleotide sequence of said second heterologous nucleic acid construct are different from each other and wherein said first blocking oligonucleotide and/or first small molecule and said second blocking oligonucleotide and/or second small molecule are different from each other.

The cell of this invention can be present outside of a subject or present inside a subject. The cell can be delivered to or introduced into the subject before, after and/or simultaneously with the delivery to or introduction into the subject of the first blocking agent (e.g., first blocking oligonucleotide and/or first small molecule) and/or the second blocking agent (e.g., second blocking oligonucleotide and/or second small molecule), in any combination. It is further intended that multiple cells comprising different first nucleotide sequences can be introduced into the same subject and/or an individual cell can comprise more than two different heterologous nucleic acid constructs, each comprising the same and/or different first nucleotide sequences in any combination, in order to regulate expression of numerous transgenes (e.g., 2, 3, 4, 5, 6, 7, 8, 9, 10, etc.) in a subject at the same time and/or at regulated times in any combination.

In addition, the present invention provides a method for producing a functional product (e.g., RNA) encoded by said first nucleotide sequence of said isolated nucleic acid of this invention, comprising contacting a blocking agent (e.g., a blocking oligonucleotide and/or small molecule) with the isolated nucleic acid under conditions as are well known in the art that permit splicing, wherein the blocking oligonucleotide and/or small molecule blocks a member of said second set of splice elements of said second intronic nucleotide sequence of said isolated nucleic acid, thereby producing the functional product (e.g., RNA) encoded by said first nucleotide sequence. In some embodiments of this method, the blocking oligonucleotide and/or small molecule can be introduced into or delivered to a cell that has been transformed by introduction or delivery of the isolated nucleic acid. The cell can be in a subject and in some embodiments, the subject can be a human, therefore in some embodiments, the isolated nucleic acid is delivered to or introduced into a cell of a subject and the blocking oligonucleotide and/or small molecule is introduced into or delivered to the cell (a cell) in which the isolated nucleic acid is present. The isolated nucleic acid can be introduced into the subject before, after and/or simultaneously with the introduction of the blocking oligonucleotide and/or small molecule. The blocking oligonucleotide can be delivered to a cell of this invention directly and/or via vector delivery and can be present in the cell either transiently or as a stably integrated sequence. Multiple blocking oligonucleotides can be introduced into the same cell via any combination of nucleic acid delivery systems and can be present in the cell in any combination of transient and stably integrated sequences.

As used herein, a “functional product encoded by said first nucleotide sequence of said first heterologous nucleic acid construct,” a “functional product encoded by said first nucleotide sequence of said second heterologous nucleic acid construct,” a “first functional product” and a “second functional product” is intended to describe the product of a functional RNA (e.g., mRNA) produced by expression of the first nucleotide sequence of the first heterologous nucleic acid construct and/or the second heterologous nucleic acid construct of this invention, as well as the product of a functional mRNA produced by expression of the first nucleotide sequence of the isolated nucleic acid of this invention. A product of a functional mRNA can be a protein or peptide encoded by said functional mRNA and thus translated from said functional mRNA into a protein or peptide. A product of a functional mRNA can also be a directly functional RNA molecule as described herein. In some embodiments, a “functional product” is intended to describe the result of regulating splicing activity in a nucleic acid construct and/or an isolated nucleic acid of this invention such that the product encoded by the first nucleotide sequence is produced (e.g., under conditions in which the first set of splice elements is active and the second set of splice elements is not active).

A blocking oligonucleotide of this invention can be single stranded or double stranded nucleic acid (RNA and/or DNA) and can be a modified nucleic acid. A blocking oligonucleotide can be a sense sequence, an antisense sequence, miRNA, siRNA, shRNA etc., as are known in the art to have blocking activity.

In various embodiments of the methods of the present invention, the blocking oligonucleotide does not activate RNase H. In various embodiments, the blocking oligonucleotide can comprise a modified internucleotide bridging phosphate residue selected from the group consisting of methyl phosphorothioates, phosphoromorpholidates, phosphoropiperazidates, phosphoramidates and any combination thereof. In further embodiments, the blocking oligonucleotide can comprise a nucleotide having a loweralkyl substituent at the 2′ position thereof and in yet further embodiments, the blocking oligonucleotide can be from about eight to about 50 nucleotides in length.

Numerous systems available, for example, from known mutated intron systems, can be employed to make the compositions of this invention and to carry out the methods of this invention. For example, the β-globin mutated intron that causes certain thallesemias can be employed (e.g., SEQ ID NO:303 (850 nt. beta-globin intron with 705G mutation); SEQ ID NO:261 (850 nt. beta-globin intron with 654 C>T mutation); SEQ ID NO:262 (wild type 850 nt. beta-globin intron), with and/or without additional mutations as described herein), (see, e.g., Suwanmanee et al. “Restoration of human beta-globin gene expression in murine and human IVS2-654 thalassemic erythroid cells by free uptake of antisense oligonucleotides” Mol. Pharmacol. (2002) 62:545-553, incorporated by reference herein in its entirety). Other systems include the mutant intron of the cystic fibrosis transmembrane conductance regulator (CFTR) gene (e.g., SEQ ID NO:313 (CFTR gene exon 19); SEQ ID NO:314 (CFTR exon 19 containing 3849+10 kb C>T mutation) with and without additional mutations), (see. e.g., Accession No. NC_{—}000007, nucleotides 116907253 to 117095951 from build 36 version 1 of NCBI genome annotation; Highsmith et al. (1994) “A novel mutation in the cystic fibrosis gene in patients with pulmonary disease but normal sweat chloride concentrations” New England Journal of Medicine 331:974-980, incorporated by reference herein in its entirety).

An additional system includes mutations in the dystrophin gene (e.g., SEQ ID NO:315 (WT Mus musculus dystrophin intron 22, exon 23 and intron 23); SEQ IDS NO:316 (mdx Mus musculus dystrophin intron 22, exon 23 and intron 23) with and without additional mutations); (see, e.g., Accession No. NC_{—}000023, nucleotides 31047266 to 33267647 from build 36 version 1 of NCBI genome annotation; Tuffery-Giraud et al. (1999) “Point mutations in the dystrophin gene: evidence for frequent use of cryptic splice sites as a result of splicing defects” Human Mutation 14:359-368; Aartsma-Rus et al. (2004) “Antisense-induced multiexon skipping for Duchenne Muscular Dystrophy makes more sense” American Journal of Human Genetics 74:83-92; Chamberlain et al. (1991) “PCR analysis of dystrophin gene mutation and expression” J. Cell. Biochem. 46:255-259; Mann et al. (2001) “Antisense-induced exon skipping and synthesis of dystrophin in the mdx mouse” Proc. Natl. Acad. Sci. USA 98:42-47; Lu et al. (2003) “Functional amounts of dystrophin produced by skipping the mutated exon in the mdx dystrophic mouse” Nat. Med. 9:1009-1014; Kole et al. (2004) “RNA modulation, repair and remodeling by splice switching oligonucleotides” Acta Biochimica Polonica 51:373-378; all of the above being incorporated by reference herein in their entireties).

Yet another system that can be employed in the methods and compositions of this invention is the mutated tau gene that causes alternative splicing defects (e.g., SEQ ID NO:321 (Homo sapiens intron 9, exon 9 and intron 10)) (see, e.g., Kalbfuss et al. “Correction of alternative splicing in tau in frontotemporal dementia and Parkinsonism linked to chromosome 17” J. Biol. Chem. 276:42986-42993 (2001), incorporated by reference herein in its entirety), as well as any other such mutated gene that produces a splicing defect, as now known or later identified. Modified introns that introduce alternative splice sets can also be produced and tested according to methods well know to the ordinary artisan.

The first nucleotide sequence can encode, for example, a protein or peptide, a nucleotide sequence having interfering activity (e.g., siRNA, miRNA, shRNA, etc.), a nucleotide sequence having enzymatic activity as an RNA, a nucleotide sequence encoding a ribozyme, a nucleotide sequence encoding an antisense sequence and/or a small nuclear RNA (snRNA), in any combination. Furthermore, the first nucleotide sequence can comprise one or more mutations and in some embodiments such mutations can play a role in defining splice sites and/or modulating splicing activity.

It is also understood that the first nucleotide sequences can be the same and/or different in any combination of repeats and/or alternates in the isolated nucleic acid of this invention. Additionally, the second intronic nucleotide sequences can be the same and/or different in any combination of repeats and/or alternates in the isolated nucleic acid of this invention.

The second intronic nucleotide sequence of this invention can be a nucleotide sequence that defines an intron that comprises one or more mutations, the presence of which results in a first set of splice elements and a second set of splice elements. In some embodiments, the second intronic nucleotide sequence can be a sequence that defines an intron-exon-intron region, wherein a mutation in either the intron and/or exon region results in the presence of a first set of splice elements and a second set of splice elements. In this latter embodiment, when the second set of splice elements is active, the result is production of an RNA comprising the exon of the intron-exon-intron region.

Further provided herein is a vector comprising a nucleic acid of this invention and a cell comprising the nucleic acid or vector of this invention. In some embodiments, the vector can be, but is not limited to a nonviral vector, a viral vector and a synthetic biological nanoparticle. Nonlimiting examples of a viral vector of this invention include an AAV vector, an adenovirus vector, a lentivirus vector, a retrovirus vector, a herpesvirus vector, an alphavirus vector, a poxvirus vector a baculovirus vector and a chimeric virus vector (i.e., combining elements from two or more different viruses).

The present invention also provides various methods employing the nucleic acids of this invention. Thus, in some embodiments, the present invention provides a method for producing a functional product, comprising; a) contacting a blocking oligonucleotide (e.g., ASO or AON) with the nucleic acid of this invention under conditions that permit splicing, wherein the blocking oligonucleotide blocks a member of the second set of splice elements, resulting in removal of the first intron by splicing and production of the first RNA; and b) translating the first RNA to produce the protein or peptide and/or to produce the RNA that imparts a biological function.

The blocking oligonucleotide and/or small molecule and/or other blocking compound of this invention can be introduced into a cell comprising the nucleic acid of this invention and such a cell can be in vitro or in a subject of this invention as described herein (e.g., an animal, which can be a human).

In additional embodiments, the present invention provides a method for producing a heterologous protein, peptide and/or an RNA that imparts a biological function, comprising: a) contacting a small molecule with the cell and/or nucleic acid of this invention under conditions which permit splicing, wherein the small molecule blocks a member of the second set of splice elements, resulting in removal of the first intron and production of the first RNA; and b) translating the first RNA to produce the protein or peptide and/or to produce the RNA that imparts a biological function.

In addition, the present invention provides a method of regulating production of a heterologous protein, peptide and/or RNA that imparts a biological function in a subject, comprising: a) introducing into the subject the cell and/or nucleic acid of this invention; and b) introducing into the subject a blocking oligonucleotide and/or small molecule that blocks a member of the second set of splice elements, at a time when production of the heterologous protein, peptide and/or RNA is desired, thereby regulating production of the heterologous protein, peptide and/or RNA in the subject.

Screening methods are also provided herein, such as a method of identifying a compound that blocks a member of the second set of splice elements of the nucleic acid of this invention, comprising: a) contacting the nucleic acid of this invention with the compound under conditions that permit splicing; and b) detecting the production of the first RNA and/or the production of the second RNA, whereby the production of the first RNA identifies a compound that blocks a member of the second set of splice elements.

In certain embodiments described herein, the heterologous nucleotide sequence expression system is introduced (e.g., into a subject) in the OFF position (i.e., no or minimal heterologous nucleotide sequence expression) and contact with a blocking oligonucleotide and/or small molecule of this invention switches the system to the ON position (i.e., heterologous nucleotide sequence expression occurs). Further provided herein are methods of turning a system which is introduced (e.g., into a subject) in the ON position to the OFF position, such as a method for inhibiting production of a heterologous protein, peptide and/or RNA, comprising: a) contacting a blocking oligonucleotide and/or a small molecule with the nucleic acid of this invention under conditions which permit splicing, wherein the oligonucleotide and/or small molecule blocks a member of the first set of splice elements, resulting in removal of the second intron, thereby inhibiting production of the first RNA.

An intron is a portion of eukaryotic DNA or RNA that intervenes between the coding portions, or “exons,” of that DNA or RNA. Introns and exons are transcribed from DNA into RNA termed “primary transcript, precursor to RNA” (or “pre-mRNA”). Introns are removed from the pre-mRNA so that the protein or functional RNA encoded by the exons can be produced (the term “protein” as used herein refers to naturally occurring, wild type, or functional protein). The removal of introns from pre-mRNA and subsequent joining of the exons is carried out in the splicing process.

The splicing process is a series of reactions that are carried out on RNA after transcription (i.e., post-transcriptionally) but before translation and that are mediated by splicing factors. Thus, a “pre-mRNA” is an RNA that contains both exons and one or more introns, and a “messenger RNA (mRNA or RNA)” is an RNA from which any introns have been removed and wherein the exons are joined together sequentially so that the gene product can be produced therefrom, either by translation with ribosomes into a functional protein or by translation into a functional RNA.

The term “translation” as used herein includes the production of an amino acid chain (e.g., a peptide or polypeptide), directed by ribosomes that move along a messenger RNA comprising codons that encode the amino acid sequence. The term translation as used herein also includes the production of a functional RNA molecule (e.g., a ribozyme, antisense RNA, RNAi, snRNA, etc.) from a complementary nucleotide sequence (e.g., an exon) encoding the nucleotide sequence of the functional RNA molecule.

Introns are characterized by a set of “splice elements” that are part of the splicing machinery and are required for splicing. Introns are relatively short, conserved nucleic acid segments that bind the various splicing factors that carry out the splicing reactions. Thus, each intron is defined by a 5′ splice site, a 3′ splice site, and a branch point situated therebetween. Splice elements also comprise exon splicing enhancers and silencers, situated in exons, as well as intron splicing enhancers and silencers situated in introns at a distance from the splice sites and branch points. In addition to splice site and branch points, these elements control alternative, aberrant and constitutive splicing (e.g., resulting from a mutation).

According to embodiments of this invention, the first nucleotide sequence can be, but is not limited to, a heterologous nucleotide sequence encoding a protein or peptide, a heterologous nucleotide sequence having enzymatic activity as an RNA; a heterologous sequence having activity as an interfering RNA, a heterologous nucleotide sequence encoding a ribozyme, a heterologous nucleotide sequence encoding an antisense sequence and/or a heterologous nucleotide sequence encoding a small nuclear RNA (snRNA), in any combination.

The terms “exogenous” and/or “heterologous” as used herein can include a nucleotide sequence that is not naturally occurring in the nucleic acid construct and/or delivery vector (e.g., virus delivery vector) in which it is contained and can also include a nucleotide sequence that is placed into a non-naturally occurring environment and/or non-naturally occurring position relative to other nucleotide sequences (e.g., by association with a promoter or coding sequence with which it is not naturally associated). Furthermore, the first nucleotide sequence of this invention can be heterologous to the second intronic nucleotide sequence of this invention, i.e., the first nucleotide sequence and second intronic nucleotide sequence do not occur together or are not operably associated with one another in a naturally occurring state. To illustrate, as one nonlimiting example of this invention, a beta-globin intron, functioning as a second intronic nucleotide sequence of this invention is introduced at one or more sites in the luciferase coding sequence, the latter of which is functioning as a first nucleotide sequence. The beta-globin intron would not occur, or be operably associated, with a luciferase coding sequence in a naturally occurring state.

In some embodiments, the first nucleotide sequence of this invention can encode a protein, peptide and/or RNA of this invention that is exogenous or heterologous [i.e., not naturally occurring, not present in a naturally occurring state and/or modified and/or duplicated (e.g., in a cell that also produces its own endogenous version of the protein, peptide and/or RNA)] to the cell into which it is introduced. The first nucleotide sequence can also be exogenous or heterologous to the vector (e.g. a viral vector) into which it is placed. Furthermore, the second intronic nucleotide sequence can be exogenous or heterologous to the vector into which it is placed and/or with respect to the first nucleotide sequence with which it is associated as an intron and/or with respect to the cell into which it is placed.

Alternatively, the protein, peptide or RNA encoded by the first nucleotide sequence can comprise, consist essentially of, or consist of a nucleotide sequence that is endogenous to the cell (i.e., one that occurs naturally in the cell) but is introduced into and/or is present in the cell as an isolated heterologous nucleic acid. By “isolated nucleic acid” is meant a nucleic acid that is substantially or essentially free from components normally found in association with the nucleic acid in its natural state. Such components include other cellular material, culture medium from recombinant production, and/or various chemicals used in chemically synthesizing the nucleic acid. An “isolated” nucleic acid of the present invention is generally free of nucleic acid sequences that flank the nucleic acid of interest in the genomic DNA of the organism from which the nucleic acid was derived (such as coding sequences present at the 5′ or 3′ ends). However, the nucleic acid of this invention can include some additional bases or moieties that do not deleteriously affect the basic characteristics of the nucleic acid.

By “isolated” protein or peptide of this invention is meant a protein or peptide that is substantially free from components normally found in association with the peptide or protein in its natural state.

By “isolated” cell is meant is a cell that has been separated from other components with which it is normally associated in nature or a cell in a subject that has been transformed, e.g., by the introduction of heterologous nucleic acid into the cell. Such a cell can be introduced into a subject or may already be present in the subject and becomes transformed by introduction of the heterologous nucleic acid into the subject, where it is taken up or internalized by a cell of the subject. For example, an isolated cell can be a cell in culture medium and/or a cell in a pharmaceutically acceptable carrier of this invention and/or a cell in a subject of this invention. By “transformed” cell is meant a cell into which a heterologous nucleic acid has been introduced by any of a variety of art-known methods for delivering to or introducing into a cell a heterologous nucleic acid. A transformed cell and/or isolated cell of this invention can be introduced into or delivered into a subject of this invention. Furthermore, a cell present in a subject can be “transformed” according to this invention by delivering to or introducing into the cell a heterologous nucleic acid of this invention.

A functional product of this invention can be an RNA (e.g., a messenger RNA or mRNA), a protein, a peptide, a ribozyme, RNAi, snRNA, an antisense RNA and the like. Thus, in some embodiments, an RNA that imparts a biological function is an RNA that is translated into a protein or peptide that imparts a biological function or it is an RNA that is produced, and/or functions directly as, an RNA that imparts a biological function as described herein (e.g., a ribozyme, RNAi, snRNA, an antisense RNA, etc.)

Nonlimiting examples of a nucleic acid construct and/or isolated nucleic acid of this invention include a nucleic acid comprising, consisting essentially of and/or consisting of the nucleotide sequence as set forth in SEQ ID NO:243 (plasmid TRCBA-int-luc (mut)), SEQ ID NO:244 (plasmid TRCBA-int-luc (wt)), SEQ ID NO:245 (plasmid TRCBA-int-luc (657GT)), SEQ ID NO:246 (plasmid GL3-int-Luc (mut)), SEQ ID NO:247 (GL3-int-Luc (wt)), SEQ ID NO:248 (GL3-int-Luc (657GT)), SEQ ID NO:249 (GL3-2int-fron-sph (mut)), SEQ ID NO:250 (GL3-3int-2fron-sph (mut)), SEQ ID NO:251 (GL3-int-Luc A (mut)), SEQ ID NO:252 (GL3-int-Luc B)), SEQ ID NO:253 (GL3-int-Luc C), SEQ ID NO:254 (GL3-int-fron (mut)), SEQ ID NO:255 (GL3-2int-sph (mut)), SEQ ID NO:256 (GL3-2int-Sph-C), SEQ ID NO:257 (GL3-sint200-sph (mut)), SEQ ID NO:258 (GL3-sint200-sph (657 GT)), SEQ ID NO:259 (GL3-sint425-sph) and/or SEQ ID NO:260 (TRCBA-int-AAT-654CT) in any combination, into which has been introduced a mutated intron of this invention, in place of the corresponding intron. Such a mutated intron of this invention can be introduced into any of these SEQ ID NOs:243-260 singly, or in multiples and/or in any combination relative to one another and relative to SEQ ID NOS:243-260. A mutated intron of this invention includes an intron having the nucleotide sequence as set forth in any of SEQ ID NOs:1-242 as well as an intron as described in any of SEQ ID NOS:243-260 with any of the mutations as shown in any of to SEQ ID NOS:1-242 incorporated therein, in any combination.

Also provided are nonlimiting examples of functional regions of these sequences as described herein (e.g., the intron and coding sequence of SEQ ID NOS:243-260 (i.e., SEQ ID NOS:264-277), an intron comprising the 654C-T mutation (SEQ ID NO:261), a wild type intron (SEQ ID NO:262) an intron comprising the 654C-T mutation and the 657TA-GT mutation (SEQ ID NO:263) and the intron and coding sequence of SEQ ID NO:260 (SEQ ID NO:278). A mutated intron of this invention includes an intron having the nucleotide sequence as set forth in any of SEQ ID NOs:261-278, 292-316, 320 and/or 321 as well as an intron as described in any of SEQ ID NOs:261-278, 292-316, 320 and/or 321 with any of the mutations as shown in any of SEQ ID NOs:1-242 incorporated therein, in any combination.

Thus, the nucleic acid construct and/or isolated nucleic acid of this invention can comprise, consist essentially of and/or consist of one or more than one nucleotide sequence and/or functional region thereof as identified herein as a “first nucleotide sequence.” Such first nucleotide sequences and/or functional regions can be present in any combination, including repeats of the same nucleotide sequence, in any order and in any position relative to one another and/or relative to other components of the nucleic acid and the nucleic acid construct of this invention.

The nucleic acid construct and/or the isolated nucleic acid of this invention can further comprise a promoter that directs expression of the first nucleotide sequence. Examples of a promoter that can be included in a nucleic acid construct or nucleic acid of this invention and operably associated with a first nucleotide sequence of this invention include, but are not limited to, constitutive promoters and/or inducible promoters, some nonlimiting examples of which include viral promoters (e.g., CMV, SV40), tissue specific promoters (e.g., muscle MCK), heart (e.g., NSE), eye (e.g., MSK) and synthetic promoters (SP1 elements). An example of a promoter of this invention is chicken beta actin promoter (CB or CBA). The promoter of this invention can be present in any position on the nucleic acid construct or nucleic acid of this invention where it is in operable association with the first nucleotide sequence. One or more promoters, which can be the same or different, can be present in the same nucleic acid construct or nucleic acid, either together or positioned at different locations on the nucleic acid construct or nucleic acid relative to one another and/or relative to a first nucleotide sequence and/or second intronic nucleotide sequence present on the nucleic acid construct or nucleic acid. Furthermore, an internal ribosome entry signal (IRES) and/or other ribosome-readthrough element can be present on the nucleic acid construct or nucleic acid. One or more such IRESs and/or ribosome readthrough elements, which can be the same or different, can be present in the same nucleic acid construct or nucleic acid, either together and/or at different locations on the nucleic acid construct or nucleic acid. Such IRESs and ribosome readthrough elements can be used to translate messenger RNA sequences via cap-independent mechanisms when multiple first nucleotide sequences are present on a nucleic acid construct or nucleic acid of this invention.

In embodiments of this invention wherein a promoter is present in the isolated nucleic acid and/or nucleic acid construct of this invention, the promoter can be positioned anywhere relative to the first nucleotide sequence(s) and/or second intronic nucleotide sequence(s). For example, the second intronic nucleotide sequence(s) can be positioned between the promoter and the first nucleotide sequence. Furthermore, the second intronic nucleotide sequence(s) can be positioned anywhere relative to the first nucleotide sequence. For example, the second intronic nucleotide sequence(s) can be positioned before, after and/or within the first nucleotide sequence. In some embodiments, the second intronic nucleotide sequence(s) can be positioned anywhere within the 5′ one/third of the nucleotides of the first, nucleotide sequence, anywhere within the middle one/third of the nucleotides of the first, nucleotide sequence and/or anywhere within the 3′ one/third of the nucleotides of the first nucleotide sequence. In some embodiments, the second intronic nucleotide sequence(s) can be positioned anywhere between an open reading frame and a poly(A) site in the first nucleotide sequence.

In certain embodiments wherein two or more second intronic nucleotide sequences are present in the nucleic acid construct and/or isolated nucleic acid of this invention, the second intronic nucleotide sequences can be positioned to be separated by at least about 5, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900 or 1000 nucleotides, including any number of nucleotides between 5 and 1000 not specifically recited herein.

The second intronic nucleotide sequence of this invention can comprise, consist essentially of and/or consist of a first set of splice elements defining a first intron that is removed by splicing to produce a first RNA molecule that imparts a biological function in the absence of activity at a second set of splice elements; and a second set of splice elements defining a second intron different from the first intron, wherein the second intron is removed by splicing to produce no RNA molecule and/or to produce a second RNA molecule that does not impart a biological function, when the second set of splice elements is active. In some embodiments, the second intronic nucleotide sequence of this invention can comprise one or more mutations, which can be a substitution, addition, deletion, etc.

Particular but nonlimiting examples of the second intronic nucleotide sequence of this invention can include, but are not limited to, the nucleotide sequence of any of SEQ ID NOS:1-242, in any combination and/or multiplicities. Particular examples of an isolated nucleic acid of this invention include, but are not limited to, SEQ ID NOS:1-242. Particular but nonlimiting examples of blocking oligonucleotides of this invention include the nucleotide sequences of SEQ ID NOS:279-291, 317-319, 322 and 323.

In some embodiments of this invention, in the nucleic acid construct and/or isolated nucleic acid of this invention, the first intron is a functional intron that is removed by splicing to produce a first RNA molecule that imparts a desired biological function. The biological function can be imparted directly in embodiments wherein the first nucleotide sequence encodes a functional RNA and/or imparted indirectly by translation of the first RNA molecule into a protein, peptide or production of an RNA that imparts a biological function. Such a biological function can include a therapeutic effect, including for example, gene therapy for restoration of, and/or increase in, the activity of a protein, peptide and/or RNA that is otherwise defective and/or present in insufficient or low amounts (e.g., to correct a genetic defect that results in a disease or disorder and is responsive to treatment such as gene therapy).

As described herein, in certain embodiments wherein the nucleic acid construct and/or isolated nucleic acid of this invention is present in an environment wherein splicing can occur and in the absence of a blocking agent of this invention, the second set of splice elements that define the second intron is active and the second intron is removed, resulting in no RNA production and/or the absence of production of the first RNA encoded by the first nucleotide sequence. In such embodiments, when the second intron is removed, the result can be the production of a second RNA molecule that does not impart a biological function of this invention (i.e., a nonfunctional RNA) and/or no second RNA molecule production at all.

The second intronic nucleotide sequence of this invention can be present anywhere on the nucleic acid construct and/or isolated nucleic acid molecule as a single nucleotide sequence or the second intronic nucleotide sequence can be present on the same nucleic acid construct and/or isolated nucleic acid as two or more second intronic nucleotide sequences that can be the same or different. Thus, for example, the second intronic nucleotide sequence can be present in multiples of two or more of the same and/or different nucleotide sequences that can be present in tandem, dispersed throughout the nucleic acid construct and/or isolated nucleic acid at different positions and/or both together (e.g., in tandem) and dispersed.

The isolated nucleic acid and/or nucleic acid construct of this invention can be present in a vector and such a vector can be present in a cell. Any suitable vector is encompassed in the embodiments of this invention, including, but not limited to, nonviral vectors (e.g., plasmids, poloxymers and liposomes), viral vectors and synthetic biological nanoparticles (BNP) (e.g., synthetically designed from different adeno-associated viruses, as well as other parvoviruses).

It will be apparent to those skilled in the art that any suitable vector can be used to deliver the heterologous nucleic acids of this invention. The choice of delivery vector can be made based on a number of factors known in the art, including age and species of the target host, in vitro vs. in vivo delivery, level and persistence of expression desired, intended purpose (e.g., for therapy or polypeptide or peptide or functional RNA production), the target cell or organ, route of delivery, size of the isolated nucleic acid, safety concerns, and the like.

Suitable vectors also include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Geminivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Nonlimiting examples of vectors employed in the methods of this invention include any nucleotide construct used to deliver nucleic acid into cells, e.g., a plasmid, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). The present invention also provides “targeted” virus particles (e.g., a parvovirus vector comprising a parvovirus capsid and a recombinant AAV genome, wherein an exogenous targeting sequence has been inserted or substituted into the parvovirus capsid).

Physical transduction techniques can also be used, such as liposome delivery and receptor-mediated and other endocytosis mechanisms (see, for example, Schwartzenberger et al., Blood 87:472-478, 1996). This invention can be used in conjunction with any of these and/or other commonly used nucleic acid transfer methods. Appropriate means for transfection, including viral vectors, chemical transfectants, or physico-mechanical methods such as electroporation and direct diffusion of DNA, are described, for example, in Wolff et al., Science 247:1465-1468, (1990); and Wolff, Nature 352:815-818, (1991).

Thus, administration of the nucleic acid of this invention can be achieved by any one of numerous, well-known approaches; for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and tissues, organs and/or cell types and/or populations by using the targeting characteristics of the carrier, which would be well known to the skilled artisan. These methods can be also used to deliver a vaccine to a subject. It would also be well understood that cell and tissue specific promoters can be employed in the nucleic acids of this invention to target specific tissues and cells and/or to treat specific diseases and disorders.

A cell comprising a vector and/or nucleic acid of this invention can be any cell that can contain a vector and/or nucleic acid of this invention, including but not limited to cells from muscle (e.g., smooth muscle, skeletal muscle, cardiac muscle myocytes), liver (e.g., to hepatocytes), heart, brain (e.g., neurons), eye (e.g., retinal; corneal), pancreas, kidney, endothelium, epithelium, stem cells (e.g., bone marrow; cord blood), tissue culture cells (e.g., HeLa cells) etc., as are well known in the art. A cell of this invention can be an isolated cell (e.g., in culture and/or otherwise removed or altered from the natural environment of the cell). A cell of this invention can also be a cell that is present in a subject of this invention.

In some embodiments, the nucleic acids of the present invention have a reduced level of “leakiness” when compared with other gene expression regulation systems. By “leakiness” is meant an amount of gene product or functional RNA that is produced when the system is in the OFF position. For example, in some embodiments described herein, the present system is in the OFF position when the nucleic acid of this invention has no contact with a blocking oligonucleotide, small molecule and/or other compound of this invention and thus, the first intron is not being spliced. Leakiness can be a problem in such regulatory systems but the level of leakiness can be less in some embodiments of the present system than in systems known in the art. Thus, the present invention also provides a gene expression regulation system having reduced leakiness in comparison with other gene expression regulation systems, wherein the system comprises a nucleic acid of this invention and/or a vector of this invention. The degree to which leakiness is reduced in the present system in comparison to other systems can be 5, 10, 15, 20, 25, 30, 35, 40, 45, 50, 55, 60, 65, 70, 75, 80, 85, 90, 95, or 100% less than the amount of leakiness observed in art-known systems.

As one example, the amount of leakiness of a system can be determined by employing a reporter gene coding sequence in the system and detecting the amount of reporter gene product produced when the system is in the OFF position. Any number of assays can be employed to detect reporter gene product, including but not limited to, protein detection assays such as ELISA and Western blotting and nucleic acid detection assays such as polymerase chain reaction, Southern blotting and Northern blotting. Other assays for detection of gene product can include functional assays, e.g., measurement of an amount of biological activity attributed to the gene product. The nucleic acids and methods of the present invention can be employed in comparative assays to demonstrate a reduced level of leakiness in comparison to other known gene regulation expression systems and nucleic acids employed therein.

Further provided herein are various methods of using the nucleic acids, vectors and cells of this invention. In particular, a method is provided herein for producing the first RNA of this invention, comprising; a) contacting a blocking agent of this invention with the nucleic acid construct and/or isolated nucleic acid of this invention under conditions that permit splicing, wherein the blocking agent blocks a member of the second set of splice elements, resulting in removal of the first intron by splicing and production of the first RNA.

Additionally provided is a method for producing a protein or peptide, comprising: a) contacting a blocking oligonucleotide and/or small molecule and/or other compound of this invention with the nucleic acid of this invention under conditions that permit splicing as would be well known in the art and as described in the examples provided herein, wherein the blocking oligonucleotide blocks a member of the second set of splice elements, resulting in removal of the first intron by splicing and production of the first RNA; and b) translating the first RNA to produce the protein or peptide.

In further embodiments, a method is provided for producing an RNA that imparts a biological function (e.g., a functional RNA), comprising: a) contacting a blocking oligonucleotide and/or small molecule and/or other compound of this invention with the nucleic acid of this invention under conditions that permit splicing, wherein the blocking agent blocks a member of the second set of splice elements, resulting in removal of the first intron by splicing and production of the first RNA; and b) translating the first RNA to produce the RNA that imparts a desired biological function. In some embodiments, the first RNA can act directly as an RNA that imparts a biological function and in other embodiments the first RNA can be translated into a protein or peptide that imparts a biological function.

In any of the methods described herein, the blocking agent of this invention can be introduced into a cell comprising the nucleic acid construct and/or isolated nucleic acid of this invention and such a cell can be in an animal, which can be a human, non-human mammal (dog, cat, horse, cow, etc.), avian, or other animal.

A blocking oligonucleotide of this invention is an oligonucleotide (e.g., single and/or double stranded RNA or DNA or a combination of both) that prevents splicing activity at a specific splice site. Splicing activity is prevented because the blocking oligonucleotide binds to a nucleotide sequence that is a member of the set of splice elements that direct the splicing event, thereby inhibiting the activity of the splice element, resulting in the inhibition of splicing activity. Thus, the blocking oligonucleotide can be complementary to a splice junction, a 5′ splice element, a 3′ splice element, a cryptic splice element, a branch point, a cryptic branch point, a native splice element, a mutated splice element, etc. Some nonlimiting examples of a blocking oligonucleotide of this invention include GCTATTACCTTAACCCAG (SEQ ID NO:279); specific for the 654T mutation of the 13 globin intron and GCACTTACCTTAACCCAG (SEQ ID NO:280); specific for the 657GT mutation of the (β globin intron). Other examples include oligonucleotides comprising, consisting essentially of and/or consisting of the nucleotide sequence of SEQ ID NOS:279-291, 317-319, 322 and 323. By “consisting essentially of” in the context of these oligonucleotide sequences, it is intended that the oligonucleotide can include additional nucleotides (e.g., 1, 2, 3, 4, 5, 6, 7, 8, 9, or 10 additional) at either the 3′ end or the 5′ end of the oligonucleotide sequence that do not materially effect the function or activity of the oligonucleotide (e.g., these additional nucleotides do not hybridize to the sequence complementary to the original oligonucleotide sequence).

In methods wherein a blocking oligonucleotide is employed in the methods of this invention, the blocking oligonucleotide can, in some embodiments, be an oligonucleotide that does not activate RNase H. Oligonucleotides that do not activate RNase H can be made in accordance with known techniques. See, e.g., U.S. Pat. No. 5,149,797 to Pederson et al. Such oligonucleotides, which can be deoxyribonucleotide or ribonucleotide sequences, contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous oligonucleotides that do not activate. RNase H are available.

Oligonucleotides of this invention can also be oligonucleotides wherein at least one, or all, of the internucleotide bridging phosphate residues are modified phosphates, such as methyl phosphonates, methyl phosphonothioates, phosphoromorpholidates, phosphoropiperazidates and phosphoramidates. As an additional example, every other one of the internucleotide bridging phosphate residues can be modified as described. In another non-limiting example, such oligonucleotides are oligonucleotides wherein at least one, or all, of the nucleotides contain a 2′ loweralkyl moiety (e.g., C1-C4, linear or branched, saturated or unsaturated alkyl, such as methyl, ethyl, ethenyl, propyl, 1-propenyl, 2-propenyl, and isopropyl). For example, every other one of the nucleotides can be modified as described. (See also Furdon et al., Nucleic Acids Res. 17:9193-9204 (1989); Agrawal et al., Proc. Natl. Acad. Sci. USA 87:1401-1405 (1990); Baker et al., Nucleic Acids Res. 18, 3537-3543 (1990); Sproat et al., Nucleic Acids Res. 17:3373-3386 (1989); Walder and Walder, Proc. Natl. Acad. Sci. USA 85:5011-5015 (1988).) Thus, in some embodiments, the blocking nucleotide of this invention can comprise a modified internucleotide bridging phosphate residue that can be, but is not limited to, a methyl phosphorothioate, a phosphoromorpholidate, a phosphoropiperazidate and/or a phosphoramidate, in any combination. In certain embodiments, the blocking oligonucleotide can comprise a nucleotide having a loweralkyl substituent at the 2′ position thereof.

Additional examples of modified oligonucleotides of this invention include peptide nucleic acids (PNA) and locked nucleic acids (LNA).

In a PNA, the backbone is made from repeating N-(2-aminoethyl)-glycine units linked by peptide bonds. The different bases (purines and pyrimidines) are linked to the backbone by methylene carbonyl linkages. Unlike DNA or other DNA analogs, PNAs do not contain any pentose sugar moieties or phosphate groups. PNAs are depicted like peptides with the N-terminus at the first (left) position and the C-terminus at the right.

The PNA backbone is not charged and this confers to this polymer a much stronger binding between PNA/DNA strands than between PNA strands and DNA strands. This is due to the lack of charge repulsion between PNA and DNA strands.

Early experiments with homopyrimidine strands have shown that the Tₘ of a 6-mer PNA T/DNA dA was determined to be 31° C. in comparison to a DNA dT/DNA dA 6-mer duplex that denatures at a temperature less than 10° C.

PNAs with their peptide backbone bearing purine and pyrimidine bases are not a molecular species easily recognized by nucleases or proteases. They are thus resistant to enzyme degradation. PNAs are also stable over a wide pH range. Because they are not easily degraded by enzymes, the lifetime of these polymers is extended both in vitro and in vivo. In addition, the fact that they are not charged facilitates their crossing through cell membranes and their stronger binding properties should decrease the amount of oligonucleotide needed for the regulation of gene expression.

LNAs are a class of nucleic acids containing nucleosides whose major distinguishing characteristic is the presence of a methylene bridge between the 2′-O and 4′-C atoms of the ribose ring. This bridge restricts the flexibility of the ribofuranose ring of the nucleotide analog and locks it into the rigid bicyclic N-type conformation. Furthermore, LNA induces adjacent DNA bases to adopt this conformation, resulting in the formation of the more thermodynamically stable form of the A duplex LNA nucleosides containing the four common nucleobases that appear in DNA (A,T,G,C) that can base-pair with their complementary nucleosides according to standard Watson-Crick rules. LNA can be mixed with DNA and/or RNA, as well as other nucleic acid analogs using standard phosphoramidite DNA synthesis chemistry. Therefore, LNA oligonucleotides can easily be tagged with, e.g., amino-linkers, biotin, fluorophores, etc. Thus, a very high degree of freedom in the design of primers and probes exists. Their locked conformation increases binding affinity for complementary sequences and provides a new chemical approach to optimize and fine tune primers and probes for sensitive and specific detection of nucleic acids. This difference is observable experimentally as an increased thermal stability of LNA-NA heteroduplexes and is dependent both on the number of LNA nucleosides present in the sequence, as well as the chemical nature of the nucleobases employed. This experimental difference can be exploited to modulate the specificity of oligonucleotide probes designed to detect specific nucleic acids targets through standard hybridization techniques.

As used herein, “a member of the second set of splice elements” includes any element that is involved in activation of splicing of the second intron. For example, an element of the second set of splice elements can be the result of a mutation in the native DNA and/or pre-mRNA that can be a substitution mutation and/or an addition mutation and/or a deletion mutation that creates a new splice element. The new splice element is thus one member of a second set of splice elements that define a second intron. The remaining members of the second set of splice elements can also be members of the set of splice elements that define the first intron. For example, if the mutation creates a new, second 3′ splice site which is both upstream from (i.e., 5′ to) the first 3′ splice site and downstream from (i.e., 3′ to) a first branch point, then the first 5′ splice site and the first branch point can serve as members of both the first set of splice elements and the second set of splice elements.

In some situations, the introduction of a second set of splice elements can cause native regions of the RNA that are normally dormant, or play no role as splicing elements, to become activated and serve as splicing elements. Such elements are referred to as “cryptic” elements. For example, if a new 3′ splice site is introduced, which is situated between the first 3′ splice site and the first branch point, it can activate a cryptic branch point between the new 3′ splice site and the first branch point.

In other situations, the introduction of a new 5′ splice site that is situated between the first branch point and the first 5′ splice site can further activate a cryptic 3′ splice site and a cryptic branch point sequentially upstream from the new 5′ splice site. In this situation, the first intron becomes divided into two aberrant introns, with a new exon situated therebetween.

Further, in some situations where a first splice element (particularly a branch point) is also a member of the set of second splice elements, it can be possible to block the first element and activate a cryptic element (i.e., a cryptic branch point) that will recruit the remaining members of the first set of splice elements to force correct splicing over incorrect splicing. Note further that, when a cryptic splice element is activated, it can be situated in either the intron and/or in one of the adjacent exons.

Thus as indicated above, depending on the set of splice elements that make up the “second set of splice elements,” the blocking oligonucleotide, small molecule and/or other compound of this invention can block a variety of different splice elements to carry out the instant invention. For example, it can block a mutated element, a cryptic element, a native element, a 5′ splice site, a 3′ splice site, and/or a branch point. In general, it will not block a splice element which also defines the first intron, of course taking into account the situation where blocking a splice element of the first intron activates a cryptic element which then serves as a surrogate member of the first set of splice elements and participates in correct splicing, as discussed above.

The length of the blocking oligonucleotide (i.e., the number of nucleotides therein) is not critical so long as it binds selectively to the intended location, and can be determined in accordance with routine procedures. Thus, in some embodiments, the blocking oligonucleotide of this invention can be between about 5 and about 100 nucleotides in length. In particular, a blocking nucleotide of this invention can be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 250, 300, 350, 400, 450 or 500 nucleotides in length. In some embodiments the blocking oligonucleotide of this invention is from eight to 50 nucleotides in length. In yet other embodiments of this invention, the blocking oligonucleotide is 15-25 nucleotides in length and can also be 18-20 nucleotides in length. A blocking oligonucleotide can be used in a method described herein as a population of identical oligonucleotides and/or as a population of different oligonucleotides present in any combination and/or in any ratio relative to one another.

A small molecule of this invention is an active chemical compound that can be structurally and/or functionally diverse in comparison with other small molecules and that has a low molecular weight (e.g., ≦5000 Daltons). A small molecule can be a natural or synthetic substance. It can be synthesized by organic chemistry protocols and/or isolated from natural sources, such as plants, fungi and microbes. A small molecule can be “drug-like” (e.g., aspirin, penicillin, chemotherapeutics), toxic and/or natural. A small molecule drug can be one or more active chemical compounds, optimally formulated as an orally available pill (including via inhalation), that interact with a specific biological target, such as a receptor, enzyme or ion channel, to provide a therapeutic effect. Specific but nonlimiting examples of a small molecule of this invention include antibiotics, nucleoside analogs (e.g., toyocamycin) and aptamers (e.g., RNA aptamers; DNA aptamers).

A small molecule of this invention can be a small molecule present in any number of small molecule libraries, some of which are available commercially. Nonlimiting examples of libraries that can contain a small molecule of this invention include small molecule libraries obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan et al. “Stereoselective Synthesis of Over Two Million Compounds Having Structural Features Both Reminiscent of Natural Products and Compatible with Miniaturized Cell-Based Assays” Am. Chem. Soc. 120, 8565-8566, 1998; Floyd et al. Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc.

The small molecules and other compounds of this invention can operate by a variety of mechanisms to modify a splicing event in the nucleic acid of this invention. For example, the small molecules and other compounds of this invention can interfere with the formation and/or function and/or other properties of splicing complexes, spliceosomes, and their components such as hnRNPs, snRNPs, SR-proteins and other splicing factors or elements, resulting in the prevention and/or induction of a splicing event in a pre-mRNA molecule. As another example, the small molecules and other compounds of this invention can prevent and/or modify transcription of gene products, which can include, for example, but are not limited to, hnRNPs, snRNPs, SR-proteins and other splicing factors, which are subsequently involved in the formation and/or function of a particular spliceosome. The small molecules and other compounds of this invention can also prevent and/or modify phosphorylation, glycosylation and/or other modifications of gene products, including but not limited to, hnRNPs, snRNPs, SR-proteins and other splicing factors, which are subsequently involved in the formation and/or function of a particular spliceosome. Additionally, the small molecules and other compounds of this invention can bind to and/or otherwise affect specific pre-mRNA so that a specific splicing event is prevented or induced via a mechanism that does not involve basepairing with RNA in a sequence-specific manner.

The present invention further provides a method of producing a protein and/or an RNA that imparts a biological function (e.g., a functional RNA) in a subject, comprising: a) introducing into the subject the nucleic acid, the vector and/or the cell of this invention; and b) introducing into the subject a blocking agent of this invention that blocks a member of the second set of splice elements, thereby producing the protein, peptide and/or RNA that imparts a biological function in the subject.

Additionally provided is a method of regulating production of a protein, peptide and/or RNA in a subject, comprising: a) introducing into the subject the nucleic acid, the vector and/or the cell of this invention; and b) introducing into the subject a blocking agent of this invention that blocks a member of the second set of splice elements, at a time when production of the protein, peptide and/or RNA is desired, thereby regulating production of the protein, peptide and/or RNA in the subject. The amount of protein, peptide and/or RNA present in a subject can be monitored over time according to art-known methods and when the amount falls below a desired and/or therapeutic level, the blocking agent can be introduced into the subject to increase production of the protein, peptide and/or RNA, thus regulating the production.

In the methods described herein wherein the nucleic acid, vector and/or cell of this invention is administered to a subject, the nucleic acid, vector and/or cell can initially be present in the subject in the absence of a blocking agent, the presence of which would result in blocking of a member of the second set of splice elements. In certain embodiments, the second set of splice elements is active and there is no or minimal (e.g., insignificant) production in the subject of the exogenous protein, peptide and/or RNA, as encoded by the first nucleotide sequence. When the blocking agent of this invention is present in the subject, a member of the second set of splice elements on the nucleic acid is blocked, resulting in removal of the first intron by splicing and subsequent production, in the subject, of the protein, peptide and/or RNA encoded by the first nucleotide sequence.

The blocking oligonucleotide, small molecule and/or other compound can be introduced into the subject at any time relative to the introduction into the subject of the nucleic acid, vector and/or cell of this invention. For example, the blocking oligonucleotide, small molecule and/or other compound can be introduced into the subject before, simultaneously with and/or after introduction of the nucleic acid, vector and/or cell into the subject. Furthermore, the blocking oligonucleotide, small molecule and/or other compound can be administered one time or at multiple times over any time interval and can extend to throughout the lifespan of the subject.

Thus, in some embodiments, the present invention provides a method of treating a disease or disorder in a subject, comprising: a) introducing into the subject an effective amount of the nucleic acid, vector and/or the cell of this invention; and b) introducing into the subject an effective amount of a blocking oligonucleotide, small molecule, and/or other compound of this invention, thereby treating the disorder in the subject. When the nucleic acid, vector and/or cell and the blocking oligonucleotide, small molecule and/or other compound are present in the subject, they are present under conditions whereby the blocking oligonucleotide, small molecule and/or other compound can contact the nucleic acid and block a member of the second set of splice elements, thereby resulting in the production of a protein, peptide and/or RNA that imparts a biological function in the subject

In additional embodiments of this invention, regulation of gene expression according to the methods of this invention can occur in the reverse of the system described herein. Specifically, in some embodiments of this invention, the system is in the “OFF” position as described herein in the absence of a blocking agent that regulates splice-mediated expression (e.g., no first RNA is produced, leading to no production of a protein, peptide and/or RNA encoded by the first nucleotide sequence). In certain other embodiments, the system of this invention can be in the “ON” position in the absence of a blocking agent that regulates splice-mediated expression. In such latter embodiments, the methods of this invention can be carried out whereby a nucleic acid, vector and/or cell of this invention that is present under conditions that result in the removal of the first intron and production of the first RNA is contacted with a blocking oligonucleotide, small molecule and/or other compound of this invention, resulting in blocking of a member of the first set of splice elements, thereby resulting in the splicing and removal of the second intron, thus producing no second RNA molecule and/or a second RNA molecule that is not encoded by the first nucleotide sequence.

An “effective amount” of a nucleic acid, vector, cell, blocking oligonucleotide, small molecule and/or other compound of this invention refers to a sufficient amount to provide a desired effect, which can be a beneficial and/or therapeutic effect. As is well understood in the art, the exact amount required will vary from subject to subject, depending on age, gender, species, general condition of the subject, the severity of the condition being treated, the particular agent administered, the site and method of administration and the like. An appropriate “effective” amount in any individual case may be determined by one of ordinary skill in the art by reference to the pertinent texts and literature (e.g., Remington's Pharmaceutical Sciences (latest edition) and/or by using routine pharmacological procedures.

“Treat” or “treating” as used herein refers to any type of treatment that imparts a benefit, which can be a therapeutic benefit, to a subject that is diagnosed with, at risk of having, suspected to have and/or likely to have a conditions (e.g., a disease, syndrome or disorder) that can be responsive in a positive way to a protein, peptide and/or RNA of this invention. A benefit can include an improvement in the condition of the subject (e.g., in one or more symptoms), delay and/or reversal in the progression of the condition, prevention or delay of the onset of the disease, syndrome or disorder, etc.

As noted herein, the present invention provides a method of treating a disorder, syndrome or disease of this invention comprising: a) introducing into the subject an effective amount of the nucleic acid of this invention; and b) introducing into the subject an effective amount of a blocking oligonucleotide and/or small molecule of this invention, thereby treating the disorder or disease in the subject.

The disease, syndrome or disorder that can be treated by a method of this invention can include any disease, syndrome or disorder that is responsive to treatment involving the presence and/or increase in amount in a subject of a protein, peptide and/or RNA of this invention that imparts a biological function. Such proteins, peptides and/or RNAs can be present in a subject via the introduction into the subject of a nucleic acid, vector and/or cell of this invention and introduction into the subject of a blocking oligonucleotide, small molecule and/or other compound of this invention.

Nonlimiting examples of diseases, syndromes and/or disorders that can be treated by methods of this invention and some examples of the gene product that can be encoded by the first nucleotide sequence of this invention and that can impart a therapeutic effect include metabolic diseases such as diabetes (insulin), growth/development disorders (growth hormone; zinc finger proteins that regulate growth factors), blood clotting disorders (e.g., hemophilia A (Factor VIII); hemophilia B (Factor IX)), central nervous system disorders (e.g., seizures, Parkinson's disease (glial derived neurotrophic factor (GDNF) and GDNF-like growth factors), Alzheimer's disease (nerve growth factor, GDNF and GDNF-like growth factors), amyotrophic lateral sclerosis, demyelination disease), bone allograft (bone morphogenic protein 2 (proteins 1-9, e.g., MBP2)), inflammatory disorders (e.g., arthritis, autoimmune disease), obesity, cancer, cardiovascular disease (e.g., congestive heart failure (phospholamban and genes related to Ca⁺⁺ pump)), macular degeneration (pigment epithelium derived factor (PDEF), β-thalassemia, α-thalassemia, Tay-Sachs syndrome, phenylketonuria, cystic fibrosis and/or viral infection. Furthermore, an intron of this invention that can be utilized to produce a second intron nucleotide sequence of this invention can be derived from a gene identified as having an intron comprising aberrant splice site(s) (e.g., as associated with β-thalassemia, α-thalassemia, Tay-Sachs syndrome, phenylketonuria, cystic fibrosis and/or viral infection), as would be known in the art.

Additional examples include nucleic acids encoding soluble CD4, used in the treatment of AIDS and α-antitrypsin (AAT), used in the treatment of emphysema caused by a-antitrypsin deficiency. Other diseases, syndromes and conditions that can be treated by the methods and compositions of this invention include, for example, adenosine deaminase deficiency, sickle cell deficiency, brain disorders such as Huntington's disease, lysosomal storage diseases, Gaucher's disease, Hurler's disease, Krabbe's disease, motor neuron diseases such as dominant spinal cerebellar ataxias (examples include SCA1, SCA2, and SCA3), thalassemia, hemophilia, phenylketonuria, and heart diseases, such as those caused by alterations in cholesterol metabolism, and defects of the immune system. Other diseases that can be treated by these methods include metabolic disorders such as, musculoskeletal diseases, cardiovascular disease and cancer. The nucleic acids of this invention can also be delivered to airway epithelia to treat genetic diseases such as cystic fibrosis, pseudohypoaldosteronism, and immotile cilia syndrome, as well as non-genetic disorders (e.g., bronchitis, asthma, COPD). The nucleic acids of this invention can also be delivered to alveolar epithelia to treat genetic diseases like α-1-antitrypsin, as well as pulmonary disorders (e.g., treatment of pneumonia and emphysema pulmonary fibrosis, pulmonary edema; delivery of nucleic acid encoding surfactant protein to premature babies or patients with ARDS).

In general, the nucleic acids and vectors of the present invention can be employed to deliver any nucleic acid with a biological function to treat or ameliorate the symptoms associated with any disorder related to gene expression. Illustrative disease states include, but are not limited to: cystic fibrosis (and other diseases of the lung), hemophilia A, hemophilia B, thalassemia, anemia and other blood disorders, AIDS, cancer (e.g., brain tumors), diabetes mellitus, muscular dystrophies (e.g., Duchenne, Becker), Gaucher's disease, Hurler's disease, adenosine deaminase deficiency, glycogen storage diseases and other metabolic defects, mucopolysaccharide disease, and diseases of solid organs (e.g., brain, liver, kidney, heart, lung, eye), and the like.

In certain embodiments, the delivery vectors of the invention may be administered to treat diseases of the CNS, including genetic disorders, neurodegenerative disorders, psychiatric disorders and/or tumors. Illustrative diseases of the CNS include, but are not limited to, Alzheimer's disease, Parkinson's disease, Huntington's disease, Rett Syndrome (e.g., by regulating expression of a vector of this invention encoding MeCP2), Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Disorders of the CNS that can be treated according to the methods of this invention include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Ocular neovascularization is the most common cause of blindness and visual disability in the US and other developed countries [43]. There are two types of ocular neovascularization that affect the retina, retinal and subretinal neovascularization. Retinal neovascularization occurs on the inner surface of the retina and grows into the vitreous [44]. It causes loss of vision by vitreous hemorrhage and by causing scar tissue on the retinal surface and in the vitreous, which exerts traction on the retina and detaches it. Subretinal neovascularization consists of new vessels growing beneath the retina and/or the retinal pigmented epithelium (RPE). The RPE usually becomes incompetent, causing serous retinal detachment.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors can also be co-delivered, either intraocularly (e.g., intravitreally) or periocularly.

Uveitis involves inflammation. One or more anti-inflammatory factors can be administered by intraocular (e.g., vitreous or anterior chamber) administration of a nucleic acid of the invention.

Retinitis pigmentosa, by comparison, is characterized by retinal degeneration. In representative embodiments, retinitis pigmentosa can be treated by intraocular (e.g., vitreal) administration of a delivery vector encoding one or more neurotrophic factors.

Age-related macular degeneration involves both angiogenesis and retinal degeneration. This disorder can be treated by administering the nucleic acid of this invention encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, preferably intravitreally.

In other embodiments, the present invention can be used to treat seizures, e.g., to reduce the onset, incidence and/or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

As a further example, somatostatin (or an active fragment thereof) can be administered to the brain using a delivery vector of the invention to treat a pituitary tumor. According to this embodiment, the delivery vector encoding somatostatin (or an active to fragment thereof) can be administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

The present invention also provides methods for screening compounds for the ability to modulate splicing events in the nucleic acid constructs and/or isolated nucleic acids of this invention. Thus, in additional embodiments, the present invention provides a method of identifying a compound that blocks a member of the second set of splice elements of the nucleic acid construct and/or isolated nucleic acid of this invention, comprising: a) contacting the nucleic acid construct and/or isolated nucleic acid with the compound under conditions that permit splicing; and b) detecting the production of the first RNA or production of the second RNA, whereby the production of the first RNA identifies a compound that blocks a member of the second set of splice elements of the nucleic acid construct and/or isolated nucleic acid of this invention and production of the second RNA identifies a compound that does not block a member of the second set of splice elements. These methods can also be employed to identify compounds that allow for increased or decreased production of the first RNA and/or of the second RNA. Compounds identified by the methods described herein can be employed in the methods of this invention, including methods of producing a protein, peptide and/or RNA that imparts a biological function as well as in methods of treatment.

In other embodiments, an alternate splicing event can be modulated by employing the oligonucleotides, small molecules and/or compounds of this invention. For example, a nucleic acid, vector and/or cell of this invention can be introduced into a subject along with a blocking oligonucleotide, small molecule and/or other compound of this invention to produce a first protein and/or RNA in the subject as a result of activation at a particular set of splice sets. The same nucleic acid can be engineered to encode a different protein, peptide and/or RNA in the subject by activating a different set of splice sets. The different protein, peptide and/or RNA is produced when a different blocking oligonucleotide, small molecule and/or compound of this invention is introduced into the subject. As an example, the first RNA could produce a first protein and/or RNA of interest when a first blocking agent is present and after addition of a different, second blocking agent of this invention, a second RNA can result, that produces a second protein, peptide or functional RNA of interest (e.g., an isoform of the first protein could be produced (e.g., interleukin (IL)-4 and its splice variant, IL-4Δ2). (See, e.g., Fletcher et al. “Increased expression of mRNA encoding interleukin (IL)-4 and its splice variant IL-4Δ2 in cells from contacts of Mycobacterium tuberculosis, in the absence of in vitro stimulation” Immunology 2004 August; 112(4):669-73; Minn et al. “Insulinomas and expression of an insulin splice variant” Lancet 2004 Jan. 31; 363(9406):363-7; Schlueter et al. “Tissue-specific expression patterns of the RAGE receptor and its soluble forms—a result of regulated alternative splicing?” Biochim Biophys Acta 2003 Oct. 20; 1630(1):1-6; Vegran et al. “Implication of alternative splice transcripts of caspase-3 and surviving in chemoresistance” Bull Cancer 2005 March; 92(3):219-26; Ren et al. “Alternative splicing of vitamin D-24-hydroxylase: A novel mechanism for the regulation of extra-renal 1,25-dihydroxyvitamin D synthesis” J Biol. Chem. 2005 March 23; et al. “Mutant huntington protein: a substrate for transglutaminase 1, 2, and 3” J Neuropathol Exp Neurol 2005 January; 64(1):58-65; Ding and Keller. “Splice variants of the receptor for advanced glycosylation end products (RAGE) in human brain” Neurosci Lett. 2005 Jan. 3; 373(1):67-72; Tang et al. “Transcript scanning reveals novel and extensive splice variations in human I-type voltage-gated calcium channel, Cav1.2 α1 subunit” J Biol Chem 2004 Oct. 22; 279(43):44335-43, Epub 2004 Aug. 6. All of these references are incorporated by reference herein in their entireties.)

The present invention further provides the nucleic acids, vectors and/or cells of this invention in compositions. Thus, in additional embodiments, the present invention provides a composition comprising the nucleic acid of this invention, the vector of this invention and/or the cell of this invention, in a pharmaceutically acceptable carrier. By “pharmaceutically' acceptable carrier” is meant a carrier that is compatible with other ingredients in the pharmaceutical composition and that is not harmful or deleterious to the subject. In particular, it is intended that a pharmaceutically acceptable carrier be a sterile carrier that is formulated for administration to or delivery into a subject of this invention.

Pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intralymph, intradermal, intraarticular, intrapleural, intraperitoneal, intracerebral, intraarterial, or intravenous), topical (i.e., both skin and mucosal surfaces, including airway surfaces), isolated limb perfusion and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose'containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

An effective amount of a composition of this invention will vary from composition to composition and subject to subject, and will depend upon a variety of factors such as age, to species, gender, weight, overall condition of the subject and the particular disease or disorder to be treated. An effective amount can be determined in accordance with routine pharmacological procedures know to those of ordinary skill in the art. In some embodiments, a dosage ranging from about 0.1 μg/kg to about 1 gm/kg will have therapeutic efficacy. In embodiments employing viral vectors for delivery of the nucleic acid of this invention, viral doses can be measured to include a particular number of virus particles or plaque forming units (pfu) or infectious particles, depending on the virus employed. For example, in some embodiments, particular unit doses can include about 10³, 10⁴, 10⁵, 10⁶, 10⁷, 10⁸, 10⁹, 10¹⁰, 10¹¹, 10¹², 10¹³ or 10¹⁴ pfu or infectious particles.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year and/or as necessary to control a particular condition and/or to achieve a particular effect and/or benefit. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

The compositions of this invention can be administered to a cell of a subject either in vivo or ex vivo. For administration to a cell of the subject in vivo, as well as for administration to the subject, the compositions of this invention can be administered, for example as noted above, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention can be pulsed onto dendritic cells, which are isolated or grown from a subject's cells, according to methods well known in the art, or onto bulk PBMC or various cell subfractions thereof from a subject.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced and/or transfected cells can then be infused (e.g., in a pharmaceutically acceptable carrier) or transplanted back into the subject per standard methods for the cell or tissue type. Standard methods are known for transplantation or infusion of various cells into a subject.

Formulations of the present invention may comprise sterile aqueous and non-aqueous injection solutions of the active compound, which preparations are preferably isotonic with the blood of intended recipient and essentially pyrogen free. These preparations may contain anti-oxidants, buffers, bacteriostats and solutes, which render the formulation isotonic with the blood of the intended recipient. Aqueous and non-aqueous sterile suspensions may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline or water-for-injection immediately prior to use.

In one formulation, the compounds of this invention may be contained within a lipid particle or vesicle, such as a liposome or microcrystal, which may be suitable for parenteral administration. The particles may be of any suitable structure, such as unilamellar or plurilamellar, so long as the compound is contained therein. Positively charged lipids such as N-[1-(2,3-dioleoyloxi)propyl]-N,N,N-trimethyl-ammoniummethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The pharmaceutical compositions of this invention can be used, for example, in the production of a medicament for the treatment of a disease and/or disorder as described herein.

The following sequences, in combination with a mutated intron of this invention as set forth in SEQ ID NOs:1-242 and/or as set forth in the respective sequence identifiers listed below are included in the present invention.

SEQ ID NO:243. plasmid TRCBA-int-luc mut. Nts 163-2036: CBA promoter; nts. 2739-4573: mutant intron (654 C-T); nts 4592-4813: polyA signal.

SEQ ID NO:244. plasmid TRCBA-int-luc (wt). Nts 163-2036: CBA promoter; nts. 2739-3588: wt intron (654 C); nts 2071-4573: intron in luciferase; nts 4592-4813: polyA signal.

SEQ ID NO:245. plasmid TRCBA-int-luc (657GT). Nts 163-2036: CBA promoter; nts. 2739-3588: mutant intron (654 C-T; 657 TA-GT); nts 2071-4573: intron in luciferase; nts 4592-4813: polyA signal.

SEQ ID NO:246. plasmid GL3-int-Luc (mut). Nts 48-250: SV40 promoter; nts. 948-1797: mutant intron (654 C-T); nts 2814-3035: polyA signal; nts. 280-2782: luciferase with mutant intron.

SEQ ID NO:247. plasmid GL3-int-Luc (wt). Nts 48-250: SV40 promoter; nts. 948-1797: wt intron (654 C); nts 280-2782: luciferase with intron; nts 2814-3035: polyA signal.

SEQ ID NO:248. plasmid GL3-int-Luc (657GT). Nts 48-250: SV40 promoter; nts. 948-1797: intron (654 C-T; 657TA-GT); nts 280-2782: luciferase with mutant intron; nts 2814-3035: polyA signal.

SEQ ID NO:249. plasmid GL3-2int-fron-sph (mut). Nts 48-250: SV40 promoter; nts. 251-1100; 1771-2620: mutant introns (654 C-T); nts 1103-3635: luciferase with mutant intron; nts 3637-3858: polyA signal.

SEQ ID NO:250. plasmid GL3-3int-2fron-sph (mut). Nts 48-250: SV40 promoter; nts. 251-1100; 1106-1965; 2635-3484: mutant introns (654 C-T); nts 1967-4469: luciferase with mutant intron; nts 4514-4735: polyA signal.

SEQ ID NO:251. plasmid GL3-int-luc A (mut). Nts 48-250: SV40 promoter; nts. 673-1522: intron (654 C-T); nts 280-2782: luciferase with intron; nts 2814-3035: polyA signal.

SEQ ID NO:252. plasmid GL3-int-Luc B (mut). Nts 48-250: SV40 promoter; nts. 1440-2289: intron (654 C-T); nts 280-2782: luciferase with intron; nts 2814-3035: polyA signal.

SEQ ID NO:253. plasmid GL3-int-Luc C (mut). Nts 48-250: SV40 promoter; nts. 1691-2540: intron (654 C-T); nts 280-2782: luciferase with intron; nts 2814-3035: polyA signal.

SEQ ID NO:254. plasmid GL3-int-fron (mut). Nts 48-250: SV40 promoter; nts. 251-1100: intron (654 C-T); nts 1103-2755: luciferase with intron; nts 2787-3008: polyA signal.

SEQ ID NO:255. plasmid GL3-2int-sph (mut). Nts 48-250: SV40 promoter; nts. 948-1797; 1798-2647: intron (654 C-T); nts 280-3632: luciferase with intron; nts 3664-3885: polyA signal.

SEQ ID NO:256. plasmid GL3-2int-sph C (mut). Nts 48-250: SV40 promoter; nts. 948-1797; 2541-3390: intron (654 C-T); nts 280-3632: luciferase with intron; nts 3664-3885: polyA signal.

SEQ ID NO:257. plasmid GL3-sint200-sph (mut). Nts 48-250: SV40 promoter; nts. 948-1597: intron (654 C-T); nts 280-2582: luciferase with intron; nts 2794-2835: polyA signal.

SEQ ID NO:258. plasmid GL3-sint200-sph (657 GT). Nts 48-250: SV40 promoter; nts. 948-1597: intron (654 C-T; 657 TA-GT); nts 280-2582: luciferase with intron; nts 2794-2835: polyA signal.

SEQ ID NO:259. plasmid GL3-sint425-sph. Nts 48-250: SV40 promoter; nts. 948-1373: intron (654 C-T); nts 280-2358: luciferase with intron; nts 2569-2615: polyA signal.

SEQ ID NO:260. plasmid TRCBA with alpha antitrypsin cDNA and mutant intron (654 C-T) at nts. 2866-3715.

SEQ ID NO:261. mutant intron (654 C-T).

SEQ ID NO:262. wt intron (654 C).

SEQ ID NO:263. intron with two mutations (654 C-T; 657 TA-GT).

SEQ ID NO:264. luciferase cDNA with mutant intron (654 C-T) at nts. 669-1518.

SEQ ID NO:265. luciferase cDNA with wild type intron at nts. 669-1518.

SEQ ID NO:266. luciferase cDNA with double mutant intron (C654 C-T; 657 TA-GT) at nts. 669-1518.

SEQ ID NO:267. luciferase cDNA with mutant intron (654 C-T) at nts. 1-850 and mutant intron (654 C-T) at nts. 1521-2370.

SEQ ID NO:268. luciferase cDNA with mutant intron (654 C-T) at nts. 1-850 and two mutant introns (654 C-T) at nts. 861-1710 and nts. 2385-3234.

SEQ ID NO:269. luciferase cDNA with mutant intron (654 C-T) at alternative location A (nts. 394-1243).

SEQ ID NO:270. luciferase cDNA with mutant intron (654 C-T) at alternative location B (nts. 1161-2010).

SEQ ID NO:271. luciferase cDNA with mutant intron (654 C-T) at alternative location C (nts. 1412-2261).

SEQ ID NO:272. luciferase cDNA with mutant intron (654 C-T) upstream of translation site (nts. 1-850).

SEQ ID NO:273. luciferase cDNA with two mutant introns (654 C-T): at nts. 669-1518 and at nts. 1519-2368.

SEQ ID NO:274. luciferase cDNA with two mutant introns (654 C-T): at nts. 669-1518 and at nts. 2262-3111.

SEQ ID NO:275. luciferase cDNA with mutant intron (654 C-T) at nts. 669-1318 and 200 base pair deletion.

SEQ ID NO:276. luciferase cDNA with double mutant intron (654 C-T; 657 TA-GT) at nts. 669-1318 and 200 basepair deletion.

SEQ ID NO:277. luciferase cDNA with mutant intron (654 C-T) at nts. 669-1094 and 425 basepair deletion.

SEQ ID NO:278. alpha antitrypsin cDNA with mutant intron (654 C-T) at nts. 772-1621.

SEQ ID NO:292 (IVS2-654 intron with 564CT mutation).

SEQ ID NO:293 (IVS2-654 intron with 657G mutation).

SEQ ID NO:294 (IVS2-654 intron with 658T mutation).

SEQ ID NO:295 (IVS2-654 intron with 657GT mutation).

SEQ ID NO:296 VS2-654 intron with 200 by deletion).

SEQ ID NO:297 (IVS2-654 intron with 425 by deletion).

SEQ ID NO:298 (IVS2-654 intron with only 197 bp).

SEQ ID NO:299 (IVS2-654 intron with only 247 bp).

SEQ ID NO:300 (IVS2-654 intron with 6A mutation).

SEQ ID NO:301 (IVS2-654 intron with 564C mutation).

SEQ ID NO:302 (IVS2-654 intron with 841A mutation).

SEQ ID NO:303 (IVS2-705 intron).

SEQ ID NO:304 (IVS2-705 intron with 564CT mutation).

SEQ ID NO:305 (IVS2-705 intron with 657G mutation).

SEQ ID NO:306 (IVS2-705 intron with 658T mutation).

SEQ ID NO:307 (IVS2-705 intron with 657GT mutation).

SEQ ID NO:308 (IVS2-705 intron with 200 by deletion).

SEQ ID NO:309 (IVS2-705 intron with 425 by deletion).

SEQ ID NO:310 (IVS2-705 intron with 6A mutation).

SEQ ID NO:311 (IVS2-705 intron with 564C mutation).

SEQ ID NO:312 (IVS2-705 intron with 841A mutation).

SEQ ID NO:313 (CFTR exon 19 wild-type sequence).

SEQ ID NO:314 (CFTR exon 19 3849+10 kb C-to-T mutation).

SEQ ID NO:315 (Mouse dystrophin intron 22, exon 23 and intron 23 wild-type sequence).

SEQ ID NO:316 (mdx Mouse dystrophin intron 22, exon 23 and intron 23 nonsense mutation).

The present invention further comprises the following oligonucleotides as nonlimiting examples of blocking oligonucleotides (e.g., ASOs or AONs) of this invention.

SEQ ID NO:317 (CFTR exon 19 wild-type oligo).

SEQ ID NO:318 (CFTR exon 19 3849+10 kb C-to-T mutation oligo).

SEQ ID NO:319 (Aritisense exon 23 skipping inducing oligo).

SEQ ID NO:279. blocking oligonucleotide GCT ATT ACC TTA ACC CAG for IVS2-654.

SEQ ID NO:280. blocking oligonucleotide GCA CTT ACC TTA ACC CAG for IVS2-654 with 657GT mutation).

SEQ ID NO:281 (oligo for 6A mutation in IVS2-654).

SEQ ID NO:282 (oligo for 564C mutation in IVS2-654).

SEQ ID NO:283 (oligo for 564CT mutation in IVS2-654).

SEQ ID NO:284 (oligo for 841A mutation in IVS2-654).

SEQ ID NO:285 (oligo for 657G mutation in IVS2-654).

SEQ ID NO:286 (oligo for 658T mutation in IVS2-654).

SEQ ID NO:287 (oligo for 705G mutation in IVS2-705).

SEQ ID NO:288 (oligo for IVS2-705).

SEQ ID NO:289 (oligo for IVS2-654).

SEQ ID NO:290 (oligo for IVS2-654).

SEQ ID NO:291 (oligo for IVS2-654).

The examples, which follow, are set forth to illustrate the present invention, and are not to be construed as limiting thereof.

### EXAMPLES

### Example 1

### Use of Alternative Splicing to Control Transgene Expression

Alternative splicing is the differential selection of which exons will be included in a mature transcript during the process of pre-messenger RNA splicing (7-10 in Man). The simplest form of alternative splicing is a choice to remove or not remove an intron (FIG. 1A). The alternative use of 5′ splice sites that will change the length of an exon (FIG. 1B) is another form. A third form is the alternative use of 3′ splice sites, which will also change the length of an exon by extending the 3′ border of the exon (FIG. 1C). A more complex yet frequent mode of alternative splicing is a choice between exon exclusion and exon skipping to (FIG. 1D). In its simplest form this choice involves one alternatively used exon in between exons that are constitutively included. One of the main features of alternative splicing is that weak splice sites are usually found on alternative exons; either a weak 3′ splice site, or a weak 5′ splice site, or both (7-10).

### Plasmids

pAAV654GFP, which was an AAV plasmid containing the GFP cDNA inserted with the mutated human β-globin intron, IVS2-654, was constructed as follows: Plasmid IVS2-654 GFP was digested with AgeI and NotI to release a fragment containing the GFP cDNA interrupted by IVS2-654 at nucleotide 105. The Agel end was filled in with Klenow enzyme prior to digestion with NotI. The isolated fragment was then inserted into the EcoRI-NotI backbone fragment of an AAV plasmid CB-AAT (Flotte, Fla.), thus generating pAAV654GFP. Plasmid pAAVwtGFP, which is a control plasmid of pAAV654GFP and contains the GFP cDNA inserted with the wild type human β-globin intron, IVS2, was constructed using the same strategy as that for the construction of pAAV654GFP.

Plasmid pGL3-Promoter was purchased from Promega Corporation (Madison, Wis.). Plasmids A-D and F were constructed by inserting IVS2-654 into the luciferase expression cassette at sites A-D and F of the pGL3-Promoter. A-D and F correspond to sites in between nucleotides 393-394, 668-669, 1160-1161, and 1411-1412 downstream of the ATG start codon, and nucleotides 3-2 upstream of the ATG, respectively. To do the intron insertion, a four-fragment ligation strategy was employed. The fragments upstream and downstream of each insertion site were amplified by PCR using a high-fidelity pfu turbo DNA polymerase (Stratagene, cat #600250), and then digested with NcoI and XbaI, respectively, to create flanking sticky ends. The two fragments were then ligated with a PCR amplified IVS2-654 intron fragment and the NcoI and XbaI double-digested pGL3-Promoter backbone fragment to generate the intron insertion plasmids. To sub-clone plasmid B into an AAV backbone, plasmid B was double digested with HindIII and XbaI to release the luciferase coding sequences. The fragment was filled in with Klenow enzyme followed by insertion into the EcoRI-NotI backbone fragment of an AAV plasmid CB-AAT (Flotte, Fla.). The resulting plasmid was named pAAV654LucB.

Plasmid pAAV654AAT was constructed by using a four-fragment ligation strategy as described above to insert the IVS2-654 into plasmid CB-AAT (Flotte, Fla.), specifically within the α1-antitrypsin (AAT) coding sequences at the site between nucleotides 770-771 downstream of the ATG start codon. Both ClaI and BamHI were used to generate the flanking sticky ends.

### AAV Vector Production and Characterization

AAV vectors used in this study were generated, purified and titered as described previously (14). A mixture of three plasmids consisting of an AAV vector plasmid, an AAV helper plasmid, XX2, and an adenovirus helper plasmid, XX6-80, was transfected into 293 cells. AAV vectors thus generated were purified with both an iodixanol gradient centrifugation and a heparin affinity chromatography (15). The physical particle titer of each AAV vector preparation was then determined using a dot-blot assay.

### Characterization of Transgene Expression In Vitro

Three marker genes, GFP, firefly luciferase and AAT, were used for studying the regulation of transgene expression in vitro using cultured cell lines in 24-well plates. For studies involving the GFP marker gene, cells were infected with 10⁴ particles of AAV vectors indicated and transfected with 8.3 pmole of ASO using the calcium phosphate transfection method. One of the ASOs used, LNA654, was a 16-mer oligonucleotide containing eight bases complementary to the alternative 5′ splice site and eight bases to the flanking sequences. This ASO was capable of selectively inhibiting the inclusion of the aberrant exon (60). The other ASO used, LNA654M, was a mismatched control of LNA654. Both LNA654 and LNA654M, synthesized by a core facility at the Department of Pharmacology, University of North Carolina, Chapel Hill, contained phosphorothioate internucleotide linkages throughout and locked nucleic acid (LNA) monomers at every other position. After transfection, the cells were cultured for another 48 hours and imaged using fluorescence microscopy. The same cells were then used for RNA isolation using the RNeasy Mini Kit (Qiagen, cat #74104). The splicing pattern of the GFP mRNA was characterized with an RT-PCR assay and electrophoresis on an 8% polyacrylamide gel as described. For studies involving the firefly luciferase and AAT marker coding sequences, cells in each 24-well were transfected with 50 ng of the corresponding plasmid and 8.3 pmole of ASO as indicated using the calcium phosphate transfection method. At 24 hours after transfection, the treated cells were harvested for luciferase assay and/or RNA isolation. To do the luciferase assay, cells in each 24-well plate were lysed with 100 μl of 1× Reporter Lysis Buffer (Promega, cat#E4030). Ten μl of the lysate was then mixed with 100 μl of luciferase substrate (Promega, cat#E4030) to determine the luciferase activity. To analyze the patterns of mRNA splicing, RT-PCR assays were performed using primers listed in Table I followed by electrophoresis on an 8% polyacrylamide gel.

### Characterization of Transgene Expression in Liver and Heart

Two markers, luciferase and AAT, were used for studying the regulation of transgene expression in 4-6 week old female Balb/c mice. For studies involving the luciferase marker, AAV vectors were targeted to the liver and heart via portal vein and direct heart injection at doses of 2×10¹¹ and 0.5×10¹¹, respectively. At 6 weeks after virus injection, the animals were imaged for basal level of luciferase transgene expression using the following procedures. Mice were anesthetized by intraperitoneal (i.p.) injection of 2.5% Avertin (0.4 mg/g body weight). Luciferin (125 ul at 25 mg/ml) was then injected i.p. into each mouse to allow for the in vivo assay of luciferase activity. The mice were then imaged using the Luciferase Imaging System (Roper Scientific) or IVIS imaging system (Xenogen). To turn on the expression of the luciferase transgene, ASO at 25 mg/kg was injected i.p. for two consecutive days. The mice were then imaged as described above at days indicated starting from the last day of ASO injection.

For studies involving the AAT marker, 2×10¹¹ particles of AAV vectors were targeted to the liver via portal vein injection. At 6 weeks post infection, blood samples were taken to determine the level of AAT expressed using a protocol described previously. To turn on the expression of the AAT transgene, ASO at 25 mg/kg was injected i.p. for two consecutive days. The AAT levels in the mice were then assayed at days indicated starting from the last day of ASO injection.

### Ocular Gene Transfer Studies

Mice were treated humanely in strict compliance with the Association for Research in Vision and Ophthalmology statement on the use of animals in research. Four week old Balb/c mice were given an intravitreous or subretinal injection of 1 μl containing 10⁹ genome particles of AAV654GFP or wild type AAVGFP with a Harvard pump apparatus and pulled glass micropipettes as previously described (26). Micropipettes were calibrated to deliver 1 μl of vehicle upon depression of a foot switch. For intravitreous injections, the adult female Balb/C mice were anesthetized, and under a dissecting microscope, the sharpened tip of the micropipette was passed through the sclera just behind the limbus into the vitreous cavity and the foot switch was depressed. Subretinal injections were performed using a condensing lens system on the dissecting microscope, with a plastic ring filled with Gonioscopic solution (Alcon, Fort Worth Tex.), which allowed visualization of the retina during the injection. The pipette tip was passed through the sclera posterior to the limbus and was positioned just above the retina. Depression of the foot switch caused a jet of injection fluid to penetrate the retina. This technique is very atraumatic and direct visualization allows for confirmation that the injection was successful, because of the appearance of a small retinal detachment (bleb).

Six weeks after injection of vector, mice were given an intravitreous injection of 1 μl containing 0.556 μg of LNA654 or LNA654M and after one day the mice were euthanized and the eyes were removed and fixed with 4% paraformaldehyde in PBS for 1 hour and with 10% phosphate-buffered formalin overnight to make flat mounts. The cornea and lens were removed and the entire retina was carefully dissected from the eyecup. Radial cuts were made from the edge to the equator of the retina and the retina was flat mounted in AQUAMOUNT mounting medium with the photoreceptor facing down. Radial cuts were also made in eyecups and they were flat mounted with the sclera facing down (choroidal flat mounts). Flat mounts were examined by fluorescence microscopy using an Axioskop microscope (Zeiss, Thornwood, N.Y.) and images were digitized using a 3 color CCD video camera (IK-TU40A, Toshiba, Tokyo, Japan) and a frame grabber.

### Results

To demonstrate the feasibility of utilizing alternative splicing to control transgene expression, the aberrantly spliced mutated intron of the human β-globin gene, IVS2-654, was inserted into a green fluorescent protein (GFP) expression cassette to produce the AAV vector designated AAV654GFP. The intron contains a C-to-T mutation at nucleotide 654 thereby activating aberrant 5′ and 3′ splicing sites that are preferably utilized over the normal unaltered splice sites during mRNA splicing. This intron has previously been shown to mediate alternative splicing in HeLa, K549, primary hematopoietic stem cells and erythroid progenitor cells, as well as in liver, colon, and small intestine in mouse [53-57].

Consequently, an alternatively used exon was inserted into the GFP coding sequences leading to mis-translation of the transgene (FIG. 2A). Correct splicing of the mutant intron can be induced by an anti-sense oligonucleotide (ASO) hybridizing to the aberrant 5′ splice site [9-12 22-25 in MAN]. One such ASO tested is LNA654 (5′-GCTATTACCTTAACCC-3′) (SEQ ID NO:359), which contains phosphorothioate internucleotide linkages throughout with alternating locked nucleic acid and deoxyribose monomers. LNA654 had no detectable toxicity when systemically administered to mice [54, 56]. Upon hybridization of the ASO, the normal splice sites are recognized and correct expression of the transgene is restored. The inserted GFP was packaged into an AAV vector to yield AAV654GFP, which was used to infect three different cell lines: 293 (ATCC Accession No. CRL-1573), a human embryonic kidney cell line; HeLa, (ATCC Accession No. CCL-2), a human cervical carcinoma cell line and U2-OS (ATCC Accession No. HTB-96), a human osteosarcoma cell line.

Subsequent transfection of the three infected cell lines with LNA654 resulted in expression of GFP as determined by fluorescence microscopy; whereas mock transfection or transfection with the control LNA654M (5′-GCAAATTCCTATTCCC-3′) (SEQ ID NO:360) did not yield any detectable GFP expression. As positive controls, the three cell lines infected with another AAV vector, AAVwtGFP, which carried a GFP expression cassette inserted with the wild type human β-globin intron-2, expressed similar corresponding levels of GFP in the absence of LNA654. To demonstrate that the LNA654 induced GFP expression was due to correction of the aberrant splicing, the above treated cells were used to extract total RNA for analyzing the splicing pattern of GFP mRNA. Treatment of cells with LNA654 converted 86%, 85% and 45% of aberrant to correct splicing in 293, HeLa and U2-OS cells, respectively (FIG. 2B, lanes 3, 7 and 11). In comparison, mock transfection or transfection with LNA654M did not yield any significant correct splicing (FIG. 2B, lanes 1-2, 5-6 and 9-10); whereas splicing of the wild type intron was completely correct (FIG. 2B, lanes 4, 8 and 12).

To determine whether the regulation system based on alternative splicing could be used to control the expression of other transgenes in addition to the GFP, the IVS2-654 intron was inserted into a firefly luciferase expression cassette. The purpose of choosing the luciferase transgene was also to allow accurate quantification of both the expression and the induction levels of transgene expression, and to determine whether the site of intron insertion affects the splicing. Thus, the IVS2-654 intron was inserted in between nucleotides 393-394, 668-669, 1160-1161 or 1411-1412 as well as at the immediately upstream of the translation start, i.e. at positions A, B, C, D and F of the luciferase expression cassette (FIG. 4A). The reason for inserting the intron upstream of the coding sequences was that the aberrant exon itself contains both an upstream ATG start codon and a downstream TAA stop codon. Therefore, inclusion of the aberrant exon at position F should prevent the synthesis of the luciferase protein; whereas correct splicing of the mutant intron induced by LNA654 would restore synthesis of the luciferase protein. The resulting constructs A-D and F were separately transfected into 293 cells. LNA654 or LNA654M was simultaneously transfected into one of the two identical sets of the cells. Twenty-four hours after the transfection, the cells were harvested for quantification of luciferase expression. For intron insertions at positions A-D, the actual levels of luciferase expression varied significantly up to 3.2-fold difference under the corresponding conditions, i.e., either in the absence or presence of LNA654 (FIG. 4B). However, the induction levels for the four constructs were comparable, ranging from 4.2 to 6.7. The similarity in the induction levels for constructs A-D indicated that flanking sequences did not dramatically influence the alternative splicing. Insertion at position F surprisingly yielded a much lower induced level of expression and a relatively high background level of expression. The low induced level could be because recognition of the 5′ alternative splice site was enhanced by the 5′ cap structure resulting in more efficient exon inclusion (9 in MAN). The high background level could be due to translation initiated at the correct start codon.

To demonstrate that the LNA654 induced luciferase expression was indeed due to correction of the aberrant splicing, another set of cells treated identically to those for the luciferase assay as described above were used to extract total RNA for analyzing the splicing pattern of the luciferase mRNA. For constructs A-D and F, treatment of cells with LNA654 converted nearly all splicing from aberrant to correct (FIG. 4C, the lower bands in lanes 2, 4, 6, 8 and 10). Interestingly, there were high basal levels of correct splicing in all of the controls (lanes 1, 3, 5, 7 and 9) compared to that for the GFP mRNA (FIG. 4C). The high basal levels of correct splicing were consistent with the relatively low levels of luciferase induction (FIG. 4C). Comparison of constructs A-D in terms of mRNA splicing indicated that the positions of intron insertion were not responsible for the high basal levels.

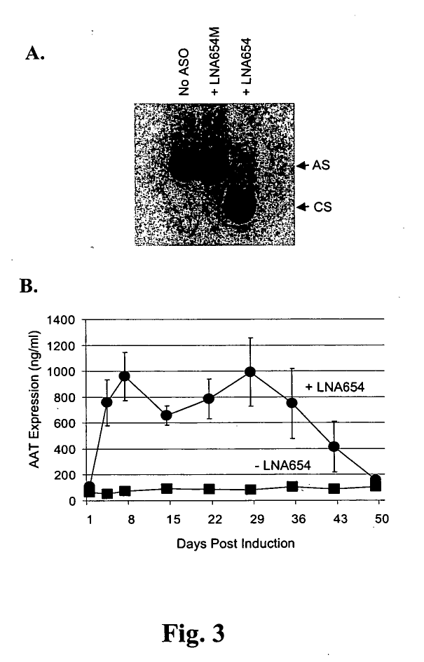

To determine whether the regulation system based on alternative splicing could be used to control the expression of another transgene, the IVS2-654 intron was inserted into an α1-antitrypsin (AAT) expression cassette to generate a construct named pAAV654AAT. The purpose of choosing the AAT transgene was to allow accurate quantification of both the expression and the induction levels of transgene expression in a mouse model. To demonstrate control of the AAT mRNA splicing, the resulting construct was transfected into 293 cells together with LNA654M or LNA654. The treated cells were then used to extract total RNA for an RT-PCR analysis. Results shown in FIG. 3A demonstrated that treatment of cells with LNA654 converted nearly all splicing from aberrant to correct in 293 cells. In comparison, transfection with LNA654M did not yield any significant correct splicing. To characterize the regulation system in an in vivo model, the pAAV654AAT construct was packaged into AAV vectors, which were targeted into mouse liver via portal vein injection.

At six weeks post infection, the mice were injected with or without LNA654 for two consecutive days. Blood samples were collected at time points indicated to monitor the induced expression of the AAT transgene. As shown in FIG. 3B, treatment of the mice with LNA654 resulted in elevated expression of AAT. The level of induced expression peaked at day 7 and maintained until day 28, followed by a gradual decrease to background level by day 49. In comparison, the mice treated without LNA654 or with the control LNA654M did not express a significant level of the AAT transgene. Altogether, these results demonstrated that alternative splicing could be used to control AAT expression both in vitro and in vivo.

To visualize the in vivo expression of a transgene regulated by alternative splicing, an AAV vector, AAV654LucB, was constructed, which carried a firefly luciferase expression cassette inserted with the IVS2-654 intron. AAV654LucB vector was targeted to mouse liver and heart via portal vein and direct heart injection, respectively. At 6 weeks post infection, the mice were injected with or without LNA654 for two consecutive days and imaged in real time for induction of luciferase expression at various time points. When the AAV was targeted to the liver, luciferase expression in the organ was induced by LNA654 administration up to 10.4 fold, peaking at day 8 and lasting more than 29 days. AAV targeted to the heart also showed a similar pattern of induced transgene expression, peaking at day 8 and lasting more than 15 days.

To test the applicability of the inducible expression system in the eye, 4-week old BALB/c mice were given a subretinal injection of AAV654GFP or AAVwtGFP. After 6 weeks, mice that had received the AAV654GFP vector were given no injection or an intravitreous to injection of LNA654 or LNA654M. The following day, the mice were euthanized and choroidal and retinal whole mounts were examined by fluorescence microscopy. Choroidal flat mounts of mice that received no injection or LNA654M showed only background fluorescence typically seen in the RPE (FIG. 6, row 1, columns 1 and 2), while choroidal flat mounts from mice injected with LNA654 showed strong fluorescence in the retinal pigmented epithelium (RPE). The fluorescence was less extensive and less intense than that seen in the RPE of mice that had received a subretinal injection of AAVwtGFP, but was still quite prominent. Retinas from mice injected with AAV654GFP that received no ASO injection or subretinal injection of LNA654M showed no fluorescence, while retinas from mice injected with LNA654 showed substantial fluorescence. At high magnification, fluorescence was observed in ganglion cells (bright dots) and their axons (arcs). Retinal fluorescence was very strong in mice injected with AAVwtGFP and high magnification showed many fluorescent ganglion cell bodies and axons. The results were similar in six eyes in each of the groups. These data indicate that the alternative splicing system of regulated expression after AAV-mediated gene transfer can be applied to the eye and that onset of reporter gene expression in the retina and RPE is detectable within a day of injection of LNA654.

### REFERENCE LIST FOR EXAMPLE 1

1. Xiao X, Li J, Samulski R J. Efficient long-term gene transfer into muscle tissue of immunocompetent mice by adeno-associated virus vector. J. Virol. 1996. 70(11):8098-108.
2. Kessler P D, Podsakoff G M, Chen X, McQuiston S A, Colosi P C, Matelis L A, Kurtzman G J, Byrne B J. Gene delivery to skeletal muscle results in sustained expression and systemic delivery of a therapeutic protein. Proc Natl Acad Sci USA. 1996. 93(24):14082-7.
3. Fisher K J, Jooss K, Alston J, Yang Y, Haecker S E, High K, Pathak R, Raper S E, Wilson J M. Recombinant adeno-associated virus for muscle directed gene therapy. Nat. Med. 1997. 3(3):306-12.
4. Nabel E G. Gene therapy for cardiovascular disease. Circulation. 1995. 91:541-8.
5. Rhee K D, Ruiz A, Duncan J L, Hauswirth W W, Lavail M M, Bok D, Yang X J. Molecular and cellular alterations induced by sustained expression of ciliary neurotrophic factor in a mouse model of retinitis pigmentosa. Invest Opthalmol Vis Sci. 2007 March; 48(3):1389-400.
6. Haberman R P, McCown T J. Regulation of gene expression in adeno-associated virus vectors in the brain. Methods. 2002 October; 28(2):219-26.
7. Lynch K W. Consequences of regulated pre-mRNA splicing in the immune system. Nat Rev Immunol. 2004. 4(12):931-40.
8. Maniatis T, Tasic B. Alternative pre-mRNA splicing and proteome expansion in metazoans. Nature. 2002. 418(6894):236-43.
9. Goldstrohm A C, Greenleaf A L, Garcia-Blanco M A. Co-transcriptional splicing of pre-messenger RNAs: considerations for the mechanism of alternative splicing. Gene. 2001. 277(1-2):31-47.
10. Lopez A J. Alternative splicing of pre-mRNA: developmental consequences and mechanisms of regulation. Annu Rev Genet. 1998. 32:279-305.
11. Spritz, R. A., Jagadeeswaran, P., Choudary, P. V., Biro, P. A., Elder, J. T., deRiel, J. K., Manley, J. L., Gefter, M. L., Forget, B. G., and Weissman, S. M. Base Substitution in an Intervening Sequence of a β+-thalassemic Human Globin Gene. Proc. Natl. Acad. Sci. U.S. A. 1981. 78:2455-2459.
12. Orkin, S. H., Kazazian, H. H., Jr., Antonarakis, S. E., Ostrer, H., Goff, S. C., and Sexton, J. P. Abnormal RNA processing due to the exon mutation of beta E-globin gene. Nature. 1982. 300:768-769.
13. Treisman, R., Orkin, S. H., and Maniatis, T. Specific transcription and RNA splicing defects in five cloned beta-thalassaemia genes. Nature 1983. 302:591-596.
14. Dobkin, C., Pergolizzi, R. G., Bahre, P., and Bank, A. Abnormal Splice in a Mutant Human β-globin Gene not at the Site of a Mutation. Proc. Natl. Acad. Sci. U.S.A. 1983. 80:1184-1188.
15. Cheng, T.-C., Orkin, S. H., Antonarakis, S. E., Potter, M. J., Sexton, J. P., Markham, A. F., Giardina, P. J., Li, A., and Kazazian, H. H., Jr. β-thalassemia in Chinese: Use of in vivo RNA Analysis and Oligonucleotide Hybridization in Systematic Characterization of Molecular Defects. Proc. Natl. Acad. Sci. U.S.A. 1984. 81:2821-2825.
16. Highsmith, W. E., Jr., Burch, L. H., Zhou, Z., Olsen, J. C., Boat, T. E., Spock, A., Gorvoy, J. D., Quittell, L., Friedman, K. J., Silverman, L. M., Boucher, R. C., and Knowles, M. R. A Novel Mutation in the Cystic Fibrosis Gene in Patients with Pulmonary Disease but Normal Sweat Chloride Concentrations. N. Engl. J. Med. 1994. 331:974-980.
17. Chillón, M., Mirk, T., Casals, T., Gimenez, J., Fonknechten, N., Will, K., Ramos, D., Nunes, V., and Estivill, X. A novel donor splice site in intron 11 of the CFTR gene, created by mutation 1811+1.6 kbA—>G, produces a new exon: high frequency in Spanish cystic fibrosis chromosomes and association with severe phenotype. Am. J. Hum. Genet. 1995. 56:623-629.
18. Lu Q L, Mann C J, Lou F, Bou-Gharios G, Morris G E, Xue S A, Fletcher S, Partridge T A, Wilton S D. Functional amounts of dystrophin produced by skipping the mutated exon in the mdx dystrophic mouse. Nat. Med. 2003. 9(8):1009-14.
19. Suwanmanee T, Sierakowska H, Lacerra G, Svasti S, Kirby S, Walsh C E, Fucharoen S, Kole R. Restoration of human beta-globin gene expression in murine and human IVS2-654 thalassemic erythroid cells by free uptake of antisense oligonucleotides. Mol Pharmacol. 2002. 62(3):545-53.
20. Friedman K J, Kole J, Cohn J A, Knowles M R, Silverman L M, Kole R. Correction of aberrant splicing of the cystic fibrosis transmembrane conductance regulator (CFTR) gene by antisense oligonucleotides. J Biol. Chem. 1999. 274(51):36193-9.
21. Kalbfuss B, Mabon S A, Misteli T. Correction of alternative splicing of tau in frontotemporal dementia and parkinsonism linked to chromosome 17. J Biol. Chem. 2001. 276(46):42986-93.
22. Lu Q L, Mann C J, Lou F, Bou-Gharios G, Morris G E, Xue S A, Fletcher S, Partridge T A, Wilton S D. Functional amounts of dystrophin produced by skipping the mutated exon in the mdx dystrophic mouse. Nat. Med. 2003. 9(8):1009-14.
23. Suwanmanee T, Sierakowska H, Lacerra G, Svasti S, Kirby S, Walsh C E, Fucharoen S, Kole R. Restoration of human beta-globin gene expression in murine and human IVS2-654 thalassemic erythroid cells by free uptake of antisense oligonucleotides. Mol Pharmacol. 2002. 62(3):545-53.
24. Friedman K J, Kole J, Cohn J A, Knowles M R, Silverman L M, Kole R. Correction of aberrant splicing of the cystic fibrosis transmembrane conductance regulator (CFTR) gene by antisense oligonucleotides. J Biol. Chem. 1999. 274(51):36193-9.
25. Kalbfuss B, Mabon S A, Misteli T. Correction of alternative splicing of tau in frontotemporal dementia and parkinsonism linked to chromosome 17. J Biol. Chem. 2001. 276(46):42986-93.
26. Mori K, Duh E, Gehlbach, P, Ando A., Takahashi K, Pearlman J, Mori K, Yang H S, Zack D J, Ettyreddy D, Brough D E, Wei L L, Campochiaro P A. Pigment epithelium-derived factor inhibits retinal and choroidal neovascularization. J Cell Physiol 2001.188, 253-263.
27. Roberts J, Palma E, Sazani P, Ørum H, Cho M, Kole R. Efficient and persistent splice switching by systemically delivered LNA oligonucleotides in mice. Mol Ther. 2006 October; 14(4):471-5.
28. Gao G P, Lu Y, Sun X, Johnston J, Calcedo R, Grant R, Wilson J M. High-level transgene expression in nonhuman primate liver with novel adeno-associated virus serotypes containing self-complementary genomes. J. Virol. 2006 June; 80(12):6192-4.

### Example 2

### Optimization of Alternative Splicing for Controlling Transgene Expression

To optimize the regulation system, the following experiments were conducted:

1) A single copy of the IVS2-654 intron was inserted at various sites within the luciferase expression cassette to control transgene expression. Both exonic and intronic sequences could modify the strength, and therefore the use, of their neighboring splice site. To determine whether insertion site within the luciferase gene affects the splicing of the intron, the IVS2-654 intron was inserted in between nucleotides 393-394, 668-669, 1160-1161 or 1411-1412 as well as immediately upstream of the translation start, i.e., at positions A, B, C, D and F of the luciferase expression cassette (FIG. 4A). The reason for inserting the intron upstream of the coding sequences was that the aberrant exon itself contains both an upstream ATG start codon and a downstream TAA stop codon. Therefore, inclusion of the aberrant exon at position F would theoretically prevent the synthesis of the luciferase protein; whereas correct splicing of the mutant intron induced by LNA654 would restore synthesis of the luciferase protein. The resulting constructs A-D and F were separately transfected into 293 cells. LNA654 or LNA654M was simultaneously transfected into one of the two identical sets of the cells. Twenty-four hours after the transfection, the cells were harvested for quantification of luciferase expression. For intron insertions at positions A-D, the actual levels of luciferase expression varied significantly, with up to 3.2-fold difference under the corresponding conditions, i.e., either in the absence or presence of LNA654 (FIG. 4B). However, the induction levels for the four constructs were comparable, ranging from 4.2 to 6.7. The similarity in the induction levels for constructs A-D indicated that flanking sequences did not dramatically influence the alternative splicing, and were therefore not the cause of the high basal level of expression. Insertion at position F yielded a much lower induced level of expression and a relatively high background level of expression.

To characterize the pattern of mRNA splicing for constructs A-D, another set of cells treated identically to those for the luciferase assay as described above were used to extract total RNA. Treatment of cells with LNA654 converted nearly all splicing from aberrant to correct. As expected, there were high basal levels of correct splicing in all of the controls, compared to that for the GFP and AAT mRNAs. The high basal levels of correct splicing were consistent with the relatively low levels of luciferase induction. Comparison of constructs A-D in terms of mRNA splicing also indicated that the positions of intron insertion were not responsible for the high basal levels. The basal level of luciferase expression in vivo is lower than that in vitro, as the induction level for AAV654LucB was 10.4 fold in the liver compared to 4.5 fold in 293 cells.

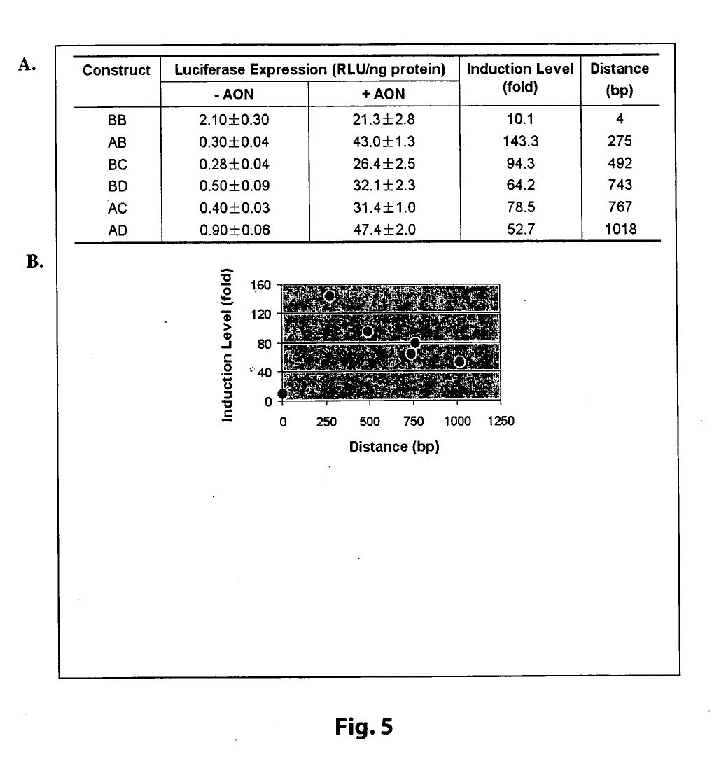

2) Two copies of the IVS2-654 intron were inserted in the luciferase expression cassette to control transgene expression. The purpose of this set of experiments was to test whether inserting two copies of the intron would improve the induction level of transgene expression and whether the distance between the two introns has any effect on the induction level. Thus, two copies of the IVS2-654 intron were placed at two different sites with various distances in between (AB, AC, AD, BC, BD and FB) or at one site in tandem (BB) (FIG. 5). The resulting plasmids were separately transfected into 293 cells with or without LNA654. Twenty-four hours after the transfection, the cells were harvested for quantification of luciferase expression. All constructs except BB led to significantly reduced levels of background expression. As a result, the induction levels were greatly improved, ranging from 10.1 to 143.3 fold. The induction levels were nearly in reverse correlation to the distance between the two introns, except in the case of two introns in tandem, i.e., the BB construct. For the BB construct, the background level of expression was significantly higher than the rest of the group. These results indicated that inserting multiple copies of the IVS-654 intron could greatly improve the induction level of transgene expression.

3) The alternative splice site of the IVS2-654 intron was modified to modulate alternative splicing. A number of different mutations were introduced in the 5′ alternative splice site (Table 3). The mutations were made to increase the strength of the alternative 5′ splice site by making the splice site more similar or identical to the consensus sequences. Compared to the 4.3 fold of induction for the parental plasmid, all mutant intron constructs resulted in improved levels of transgene induction. In particular, both the 657GT and M3 constructs yielded 223 fold and 164 fold of induction levels, respectively. These results indicated that by modulating the strength of the splice site, alternative splicing could be optimized to control transgene expression.

### Sequence of IVS2-654 Intron

The IVS2-654 intron is 850 by in size and contains four splice sites. The nucleotide sequence of the wild type IVS2-654 intron (SEQ ID NO:19) is shown below. The two alternative introns are located at nucleotides 1-579 and 653-850. The alternative exon is located at nucleotides 580-652. The two arrows mark the junctions between the alternative intron-exon. The four splice sites and the four potential branch sites are indicated by straight and curvy underlines, respectively. The target sequences of the 5′ ss 652/18 AON are in bold emboss. Sequences required for efficient splicing and 3′ end formation are in bold italic:

| | |
|---|---|
| 1 | [symbol] [symbol] [symbol] [symbol] [symbol] CCCTTCTT |
| | TTCTATGGTT AAGTTCATGT CATAGGAAGG GGAGAAGTAA |
| | CAGGGTACAG |
| 91 | TTTAGAATGG GAAACAGACG AATGATTGCA TCAGTGTGGA |
| | AGTCTCAGGA TCGTTTTAGT TTCTTTTATT TGCTGTTCAT |
| | AACAATTGTT |
| 181 | TTCTTTTGTT TAATTCTTGC TTTCTTTTTT TTTCTTCTCC |
| | GCAATTTTTA CTATTATACT TAATGCCTTA ACATTGTGTA |
| | TAACAAAAGG |
| 271 | AAATATCTCT GAGATACATT AAGTAACTTA AAAAAAAACT |
| | TTACACAGTC TGCCTAGTAC ATTACTATTT GGAATATATG |
| | TGTGCTTATT |
| 361 | TGCATATTCA TAATCTCCCT ACTTTATTTT CTTTTATTTT |
| | TAATTGATAC ATAATCATTA TACATATTTA TGGGTTAAAG |
| | TGTAATGTTT |
| 451 | TAATATGTGT ACACATATTG ACCAAATCAG GGTAATTTTG |
| | CATTTGTAAT TTTAAAAAAT GCTTTCTTCT TTTAATATAC |
| | TTTTTTGTTT |
| 541 | ATCTTATTTC TAATACTTTC CCTAATCTCT TTCTTTCAG˜G |
| | GCAATAATGA TACAATGTAT CATGCCTCTT TGCACCATTC |
| | TAAAGAATAA |
| 631 | CAGTGATAAT TTCTGGGTTA AG↓GTAATAGC AATATTTCTG |
| | CATATAAATA TTTCTGCATA TAAATTGTAA CTGATGTAAG |
| | AGGTTTCATA |
| 721 | TTGCTAATAG CAGCTACAAT CCAGCTACCA TTCTGCTTTT |
| | ATTTTATGGT TGGGATAAGG CTGGATTATT CTG [symbol] |
| | [symbol] |
| 811 | [symbol] [symbol] [symbol] [symbol] [symbol] [symbol] |

Luciferase cDNA.

In the following nucleotide sequence of a fire-fly luciferase cDNA (SEQ ID NO:320), potential sites for intron insertion are underlined. Positions A-D are indicated by both the wavy underlines and the corresponding letters on the left.

| | | |
|---|---|---|
| | 1 | ATGGAAGACG CCAAAAACAT AAAGAAAGGC |
| | | CCGGCGCCAT TCTATCCGCT GGAAGATGGA |
| | | ACCGCTGGAG AGCAACTGCA TAAGGCTATG |
| | 91 | AAGAGATACG CCCTGGTTCC TGGAACAATT |
| | | GCTTTTACAGATGCACATAT CGAGGTGGAC |
| | | ATCACTTACG CTGAGTACTT CGAAATGTCC |
| | 181 | GTTCGGTTGG CAGAAGCTAT GAAACGATAT |
| | | GGGCTGAATA CAAATCACAG AATCGTCGTA |
| | | TGCAGTGAAA ACTCTCTTCA ATTCTTTATG |
| | 271 | CCGGTGTTGG GCGCGTTATT TATCGGAGTT |
| | | GCAGTTGCGC CCGCGAACGA CATTTATAAT |
| | | GAACGTGAAT TGCTCAACAG TATGGGCATT |
| A | 361 | TCGCAGCCTA CCGTGGTGTT CGTTTCCAAA |
| | | AAGGGGTTGC AAAAAATTTT GAACGTGCAA |
| | | AAAAAGCTCC CAATCATCCA AAAAATTATT |
| | 451 | ATCATGGATT CTAAAACGGA TTACCAGGGA |
| | | TTTCAGTCGA TGTACACGTT CGTCACATCT |
| | | CATCTACCTC CCGGTTTTAA TGAATACGAT |
| | 541 | TTTGTGCCAG AGTCCTTCGA TAGGGACAAG |
| | | ACAATTGCAC TGATCATGAA CTCCTCTGGA |
| | | TCTACTGGTC TGCCTAAAGGTGTCGCTCTG |
| B | 631 | CCTCATAGAA CTGCCTGCGT GAGATTCTCG |
| | | CATGCCAGAG ATCCTATTTT TGGCAATCAA |
| | | ATCATTCCGG ATACTGCGAT TTTAAGTGTT |
| | 721 | GTTCCATTCC ATCACGGTTT TGGAATGTTT |
| | | ACTACACTCG GATATTTGAT ATGTGGATTT |
| | | CGAGTCGTCT TAATGTATAGATTTGAAGAA |
| | 811 | GAGCTGTTTC TGAGGAGCCT TCAGGATTAC |
| | | AAGATTCAAA GTGCGCTGCT GGTGCCAACC |
| | | CTATTCTCCT TCTTCGCCAA AAGCACTCTG |
| | 901 | ATTGACAAAT ACGATTTATC TAATTTACAC |
| | | GAAATTGCTT CTGGTGGCGC TCCCCTCTCT |
| | | AAGGAAGTCG GGGAAGCGGT TGCCAAGAGG |
| | 991 | TTCCATCTGC CAGGTATCAG GCAAGGATAT |
| | | GGGCTCACTG AGACTACATC AGCTATTCTG |
| | | ATTACACCCG AGGGGGATGA TAAACCGGGC |
| C | 1081 | GCGGTCGGTA AAGTTGTTCC ATTTTTTGAA |
| | | GCGAAGGTTG TGGATCTGGA TACCGGGAAA |
| | | ACGCTGGGCG TTAATCAAAGAGGCGAACTG |
| | 1171 | TGTGTGAGAG GTCCTATGAT TATGTCCGGT |
| | | TATGTAAACA ATCCGGAAGC GACCAACGCC |
| | | TTGATTGACA AGGATGGATG GCTACATTCT |
| | 1261 | GGAGACATAG CTTACTGGGA CGAAGACGAA |
| | | CACTTCTTCA TCGTTGACCG CCTGAAGTCT |
| | | CTGATTAAGT ACAAAGGCTA TCAGGTGGCT |
| D | 1351 | CCCGCTGAAT TGGAATCCAT CTTGCTCCAA |
| | | CACCCCAACA TCTTCGACGC AGGTGTCGCA |
| | | GGTCTTCCCG ACGATGACGC CGGTGAACTT |
| | 1441 | CCCGCCGCCG TTGTTGTTTT GGAGCACGGA |
| | | AAGACGATGA CGGAAAAAGA GATCGTGGAT |
| | | TACGTCGCCA GTCAAGTAAC AACCGCGAAA |
| | 1531 | AAGTTGCGCG GAGGAGTTGT GTTTGTGGAC |
| | | GAAGTACCGA AAGGTCTTAC CGGAAAACTC |
| | | GACGCAAGAA AAATCAGAGAGATCCTCATA |
| | 1621 | AAGGCCAAGA AGGGCGGAAA GATCGCCGTG TAA |

### Example 3

### Development of Small Introns for Alternative Splicing

The IVS2-654 intron is 850 base-pairs (bp) long. This size could prove to be a problem for inserting multiple copies of the intron to control transgene expression mediated by AAV. This is because the packaging limit of AAV is 4.7 kb. If two IVS2-654 introns are used for insertion, the cloning capacity would be reduced to less than 3 kb. To minimize the size of the intron, a small intron of 247 bp, termed S0 (SEQ ID NO:92), was derived from the 850 by IVS2-654 intron, which contained the four essential splice sites and the alternative exon as well as the first 32 by on the 5′ end and the last 57 by on the 3′ end that are required for efficient splicing and formation of the 3′ end of the β-globin mRNA. Insertion of the 247 by S0 intron into site A and B of the luciferase gene, yielding constructs A(S0) and B(S0), respectively, resulted in alternative splicing of the luciferase message (FIG. 4A and Table 4). Importantly, the induction levels for A(S0) and B(S0) were 4.4 and 4.3 fold, respectively, similar to that for their counterparts, constructs A and B.

To further characterize the utility of the 247 by S0 intron, the TA in the alternative splice site was converted into GT to mirror the construct 657GT. The modified plasmid, termed B(S0−GT) (SEQ ID NO:2), yielded an induction level of 228 fold. One of the two branch sites in the upstream alternative intron was also mutated in construct B(S0). The AA at nucleotides corresponding to positions 564 and 565 in IVS2-654 was converted to CT to make the upstream branch site less similar to the consensus sequences, leaving the downstream potential branch site intact. The AA→QCT mutation as in construct B(S0−CT; SEQ ID NO:1) increased the induction level from 4.3 to 24 fold. Two copies of the S0 intron were also inserted into both sites A and B of the luciferase cassette to yield A(S0)-B(S0). The resulting construct yielded an induction level of 120 fold. These achievements with the 247 by intron, which is almost four times shorter than the original IVS2-654 intron, demonstrate its utility for controlling transgene expression mediated by AAV.

### Example 4

### Development of Alternative Splicing to Differentially Regulate the Expression of Two Transgenes

To determine whether the alternative splicing system can be developed to independently control the expression of two different transgenes, the alternative splice site and its flanking sequences were mutated within the S0 intron and then the mutated intron was tested for the ability to undergo alternative splicing. In one of the mutated introns, named S1 (SEQ ID. NO:3), the original sequence of ctgGGTTAaggtaaTAgc (SEQ ID NO:376) was changed to ctgCCAATaggtaaGTgc (SEQ ID NO:377). This intron was inserted into the firefly luciferase expression cassette to yield pLucS1, which is made up of the S1 intron (SEQ ID NO:3) inserted into site B (i.e., between nucleotides 668-669) of the firefly luciferase cDNA (SEQ ID NO:320). The resulting plasmid, as well as pGFP654, which contained the IVS2-654 intron inserted into the GFP expression cassette, was used to test the strategy of differential regulation. In this experiment, pLucS1 and pGFP654 were either transfected into 293 cells separately or mixed. In each set of the transfections, the cells were also transfected with no ASO, LNA654, LNAS1 (5′-GCACTTACCTATTGGC-3′ (SEQ ID NO:361) or a mixture of the latter two. LNAS1 contained sequences complementary to the mutated alternative splice site and its flanking sequences. As shown in Table 5. LNA654 and LNAS1 independently regulated, without any crossover, the expression of GFP and luciferase, respectively. These results indicate that it is possible to differentially regulate the expression of multiple transgenes.

The overwhelming majority of 5′ and 3′ splice sites conform to the consensus sequences of ⁻²AG↓GUPuAGU⁺⁶ and ⁻⁴NPvAG↓PuN⁺², respectively, where the arrow marks the exon-intron junction and the underlined positions denote the most highly conserved residues. If splice sites are involved in alternative splicing, then they are typically less consensus. For example, the 5′ alternative splice site of the IVS2-654 intron is ⁻²AG↓GUAAUA⁺⁶, which does not have the highly conserved G at the +5 position and the consensus U at the +6 position. However, mutation of the alternative splice site to more conserved sequences of ⁻²AG↓GUAAgA⁺⁶, ⁻²AG↓GUAAUu⁺⁶, ⁻²AG↓GUAAgu⁺⁶ and ⁻²AG↓GUgAgu⁺⁶ still retains the ability of the intron to undergo alternative splicing (FIG. 6). Thus, there is some flexibility for the sequences of the 5′ alternative splice site.

In the regulation system of this invention, which is based on alternative splicing, transgene expression is controlled by using ASOs targeting the 5′ alternative splice site to modulate the alternative splicing of transgene message. As one example, the ASO, LNA654, is a 16-mer oligonucleotide complementary to both the 5′ alternative splice site and its flanking sequences. Thus, even without taking into consideration the flexibility within the 5′ alternative splice site, there are 8 (16−8=8) bases in the flanking sequences within the LNA654 target that could be mutated without affecting the strength of the alternative splice site. LNA654M, which has 6 mis-matches compared to LNA654, did not cross-modulate the alternative splicing of IVS2-654 intron. Therefore, within the LNA654 target, there are more bases than sufficient (8>6) that could be mutated to create different target sequences that would not be cross-modulated by other ASOs. In other words, it is possible to use different ASOs to independently modulate the alternative splicing of introns with different ASO targets. If multiple transgenes are each inserted with a different alternative splicing intron, it would be possible to differentially regulate the expression of the transgenes.

Assuming a mutation at each position of the 8-base flanking sequences can be any of the four nucleotides and six mis-matches would be sufficient to prevent cross-modulation by other ASO (which is the case for LNA654M, LNAS1 and LNA654), and also taking into consideration the flexibility within the 5′ alternative splice site, the number of introns that could be independently regulated by targeting the 5′ alternative splice site within the same organism is at least four and as many as eight (so that each ASO target will differ from each other by six bases, these six bases need to be considered as one because ASO targets having overlap with any one of the six bases will not qualify). Such capacity of transgene regulation would be impossible for the commonly used regulation systems such as the tet-on and the rapamycin inducible systems. In fact, these systems each can independently regulate only one transgene in theory. To achieve this specific aim, a panel of alternative splicing introns with different ASO targets will be constructed and tested for the ability to independently regulate the expression of marker transgenes in vitro.

Experimental Design. To simplify the generation of a panel of alternative splicing introns that will not be cross-modulated by other ASO, one intron will be constructed at a time instead of using a library approach. Also, the introns will be generated in the same background, i.e., all introns will be embedded in the same position of the same expression cassette. For fast, convenient and quantitative screening of the introns constructed, pLucB(S0−CT) will be used as a template for the mutations pLucB(S0−CT) has been shown to have a low basal level and high induction of expression (FIG. 7). As discussed above, the number of introns that could be independently regulated within the same organism is four or more. However, there are many sets of possible ASO targets that can be generated and tested. In fact, if the six positions within the flanking sequences are fixed at which mutations are to be introduced, then the number of possible sets of ASO targets equals 3⁶=729. To simplify the generation of mutants, two sets of introns with different ASO targets will be constructed (Table 2). If more mutants are needed for the screening, other possible sets can be generated according to the teachings provided herein and according to methods well known in the art.

To test if each construct is viable for mediating alternative splicing and is cross-modulated by other ASOs for other introns within the same set, the construct will be transfected with its specific ASO and with each of the other ASOs, respectively. By determining the change in the level of luciferase expression, suitable introns can be selected. To ensure that the introns generated and selected are indeed capable of independently regulating the expression of different transgenes, each of the introns will be inserted into one of a panel of fluorescent protein coding sequences that include green, blue and red fluorescent proteins. The resulting constructs will be mixed and transfected together with each of the corresponding ASOs. The expression of the fluorescent protein coding sequences will be determined by fluorescence microscopy using appropriate filters. A specific ASO would be expected to only induce the expression of its corresponding construct.

Generating mutations of pLucB(S0−CT). The mutations will be generated by using Stratagene's QuikChange Multi Site-Directed Mutagenesis kit. This method involves synthesis of mutant strands using primers containing desired mutations, digestion with Dpnl to remove the parental plasmid, and transformation of the synthesized single-stranded plasmids into a bacteria host to be converted into double-stranded plasmids. As an alternative method for generating mutated constructs, a pair of complementary primers containing mutations to be introduced will be used separately in a polymerase chain reaction (PCR) with another primer either upstream or downstream of the intron. PCR products from the two separate reactions will be combined as templates for another round of PCR reaction to reconstitute the mutated introns. The resulting PCR products will be digested with restriction enzymes and used to replace the corresponding fragment in the parental plasmid, thereby creating constructs containing desired mutations.

Screening for alternative splicing introns. To test if a mutant intron embedded in the luciferase coding sequence is viable for mediating alternative splicing and is cross-modulated by other ASOs for other introns within the same set, the construct will be co-transfected with its specific ASO and with each of the other ASOs, respectively. 293 cells in each 24-well plate will be transfected with 50 ng of the corresponding plasmid and 8.3 pmole of the appropriate ASO using the calcium phosphate transfection method. At 24 hours after transfection, the treated cells will be harvested and lysed with 100 μl of 1× Reporter Lysis Buffer (Promega, cat#E4030). Ten μl of the lysate will be mixed with 100 μl of luciferase substrate (Promega, cat#E4030) to measure the luciferase activity. By determining the change in the level of luciferase expression, suitable introns will be selected.

Inserting introns into fluorescent protein genes. The sequence 5′ AG Pu 3′, where Pu=G or A, will be selected within the gene or coding sequence for intron insertion. This criterion is based on the fact that the overwhelming majority of 5′ and 3′ splice site sequences conform to the consensus ⁻²AG↓GUPuAGU⁺⁶ and ⁻⁴NPyAG↓PuN⁺², respectively, where the arrow marks the exon-intron junction (Goldstrohm et al. “Co-transcriptional splicing of pre-messenger RNAs: Considerations for the mechanism of alternative splicing” Gene 2001; 277:31-47, the entire contents of which are incorporated by reference herein). Therefore, inserting an intron in between sequences 5′ AG and Pu 3′ would restore both the consensus 5′ and 3′ splice sites. To insert the introns, a recombination method will be used that was used to generate the IVS2-654 containing GFP construct (Jones et al. “A rapid method for recombination and site-specific mutagenesis by placing homologous ends on DNA using polymerase chain reaction” Biotechniques 1991: 10:62-66, the entire contents of which are incorporated by reference herein). Briefly, DNA segments are modified by using amplifying primers that add homologous ends to the PCR products. Each pair of these homologous ends would then undergo recombination to yield the desired construct following transformation of recA-E. coli. As an alternative, a four-fragment ligation strategy will be used. The fragments upstream and downstream of each insertion site are amplified by PCR using a high-fidelity pfu turbo DNA polymerase (Stratagene, Cat# 600135), and then digested with two different restriction enzymes, Enz A and Enz B, to create flanking sticky ends. The two fragments are then ligated with a PCR amplified intron fragment and an Enz A and Enz B double-digested plasmid backbone fragment to generate the intron insertion plasmids.

Other alternative splicing introns, such as the 415 by LCFSN-3849mut derived from the CFTR gene, could also be used as a template for introducing mutations into the ASO target (Friedman et al., Correction of aberrant splicing of the cystic fibrosis transmembrane conductance regulator (CFTR) gene by antisense oligonucleotides” J. Biol. Chem. 1999; 274:36193-9, the entire contents of which are incorporated by reference herein). These introns have different sequences surrounding the ASO target and may not form secondary structures that might otherwise form in the context of the S0−CT intron after mutation. To ensure that each ASO would bind tightly to its target, its length will be varied so that its Tₘ matches that of LNA654. Like LNA654, other ASOs can also have phosphorothioate internucleotide linkages throughout and alternating LNA and deoxyribose monomers.

If mis-matching at six positions is not sufficient to prevent cross-modulation, then there are still two more positions in the flanking sequences available for introducing mutations. Furthermore, there are flexibilities within the 5′ alternative splice site that can be used to introduce variations within the ASO target. Hence, it is very likely that a set of four different introns would be generated that are not cross-modulated by other ASOs. In addition to the 5′ alternative splice site, the 3′ alternative splice site can also be targeted by ASOs to modulate alternative splicing (Sierakoswka et al. “Repair of thalassemic human beta-globin mRNA in mammalian cells by antisense oligonucleotides” Proc. Natl. Acad. Sci. USA 1996; 93(23:12840-4, the entire contents of which are incorporated by reference herein). By combining approaches of targeting the 5′ and the 3′ alternative splice sites, using different alternative splicing introns as mentioned above, fine-tuning the number of mis-matches required for preventing cross-modulation by other ASOs, taking advantage of the flexibilities within both the 5′ and the 3′ splice sites, and extending the length of ASO targets to introduce mutation at more positions, it could be possible to generate a set of at least four and up to 16 different introns that are not cross-modulated by other ASOs.

With respect to the availability of sites for inserting introns, the requirement of 5′ AG Pu 3′ should be easily fulfilled. In the unlikely event of having to create such a site, the multiple codon usage for each amino acid can be exploited. For example, in the sequences of 5′ (NNX) (GPuN) 3′, where each pair of parentheses marks a codon, the nucleotide X could be converted as a silent mutation to A thereby generating the required 5′ AG Pu 3′ sequences for intron insertion. Similarly, in the sequences of 5′ (NAZ) (PuNN) 3′, nucleotide Z could be converted as a silent mutation to G. Of the twenty amino acids, eleven of them contain G and twelve of them contain A at the last position of their codons as an alternative usage. Therefore, the possibility of being able to create an intron insertion site is relatively high.

### Example 5

### Regulation of the Expression of Multiple Transgenes in Eye Models

In the present invention, the development of minimal alternative splicing introns capable of regulating transgene expression is described. Furthermore, the feasibility of using two or more different ASOs to independently control the expression of two or more different transgenes has been demonstrated. Using an AAV vector for delivering the regulation system of this invention, a marker gene has successfully been expressed in mouse eye in a controlled manner. Therefore, in the present invention, further embodiments include the use of the gene regulation system of this invention in gene therapy of ocular diseases. For example, in some embodiments, the gene regulation system described herein could be developed to independently regulate the expression of multiple transgenes. Such differential expression of multiple potent factors inhibiting angiogenesis via different pathways would be expected to have a synergistic effect in treating ocular neovascularization.

AAV as a gene transfer vector. Wild type AAV is a non-pathogenic, non-enveloped, small single-stranded DNA virus with a genome of 4.7 kilo bases (kb) [13]. Currently, at least eleven serotypes of AAV have been developed and tested as gene transfer vectors [14-16]. Each of these vectors can be distinguished by efficiency of transduction for specific target tissue. Strategies for generating different AAV serotype vectors have been developed [14, 15, 17]. Pre-clinical studies using AAV have demonstrated substantial correction, and in some instances complete cure, of genetic diseases, supporting this vector as a viable delivery system for gene therapy of ocular disorders [2]. To improve the efficiency of transgene expression, a double-stranded AAV vector has been developed, which is up to 140-fold more efficient in transduction than conventional AAV vectors [18]. Double stranded AAV also has a faster onset of gene expression and provides an extremely efficient vector for gene transfer into many types of cells in vivo [18-21]. However, this vector has a drawback of having a small cloning capacity of only 2.4 kb compared to 4.7 kb for the conventional single-stranded AAV vector. Thus, the need becomes more obvious for the availability of minimal elements to control transgene expression. AAV vectors have been demonstrated to mediate efficient transgene expression in all major organs and tissues in animals [1-3, 22]. A general feature of AAV gene transfer in vivo is that it results in long-term persistence of the vector genome, e.g., so far at least nine years in monkey, five years in dog and four years in human.

Ocular neovascularization is the most common cause of blindness and visual disability in the US and other developed countries [43]. There are two types of ocular neovascularization that affect the retina, retinal and subretinal neovascularization. Retinal neovascularization occurs on the inner surface of the retina and grows into the vitreous [44]. It causes loss of vision by vitreous hemorrhage and by causing scar tissue on the retinal surface and in the vitreous, which exerts traction on the retina and detaches it. Subretinal neovascularization consists of new vessels growing beneath the retina and/or the retinal pigmented epithelium (RPE). The RPE usually becomes incompetent, causing serous retinal detachment.

In both types of neovascularization, VEGF plays a central role in the pathogenesis of the disease. The identification of VEGF as an important therapeutic target and the development of potent VEGF antagonists have revolutionized the treatment of retinal vascular diseases. For example, previous treatments for ocular neovascularization due to age-related macular degeneration did not cause improvement and merely slowed the rate of vision loss. Intraocular injections of Ranibizumab, an antibody fragment that binds all isoforms of VEGF-A, caused significant improvement in 30-40% of patients that was sustained for at least two years [45, 46]. Despite the benefits, Ranibizumab treatment did not cause complete regression of neovascularization, and many patients require intraocular injections every month to sustain benefits. Further efforts are needed to develop more effective treatment strategies.

The development of more effective treatments depends on a clear understanding of the molecular and cellular processes involved in angiogenesis. Angiogenesis is a complex multi-step process that involves the sprouting of vascular endothelial cells from existing vessels through endothelial cell proliferation, migration, tube formation and remodeling of extracellular matrix [47, 48]. This process is controlled by complex interactions between growth factors, extracellular matrix and cellular components, the net outcome being determined by the balance of angiogenic and angiostatic elements [49]. A number of growth factor molecules are involved in the control of angiogenesis and the therapeutic manipulation of one or a combination of these offers the potential means to control neovascularization in the eye [47, 48]. Small molecules blocking both VEGF and PDGF signaling cause near complete suppression of choroidal neovascularization [50, 51]. Cytokines that have been targeted and/or angiostatic proteins that have been bolstered using a gene therapy approach in experimental models include vascular endothelial growth factor (VEGF), insulin-like growth factor-1 (IGF-1), pigment epithelium-derived factor (PEDF), matrix metalloproteinases (MMPs), angiostatin, endostatin and integrins. However, none has achieved near complete regression of neovascularization [48].

The effective control of angiogenesis in patients with retinal neovascular disorders is likely to require the long-term presence of the angiostatic protein in the eye. Although inhibition of VEGF alone has no adverse effect on normal retinal vascular development or structure [43, 48, 52], there remains the possibility that sustained and unregulated expression of multiple angiostatic proteins presents a risk of adverse local effects. In fact, there is overlap in the survival signals used by mature vascular cells and even some neurons with those used by endothelial cells of new vessels [48]. Inappropriate inhibition of neovascularization could cause damage to normal ocular structures. Therefore, development of strategies to enable appropriate regulation of gene expression is desirable to minimize the potential for local toxicity.

The ability to regulate the expression of transgenes is essential to ensure the safety of many gene therapy strategies. This is particularly the case for gene therapy of eye diseases due to neovascular disorders, which may requires long-term presence of multiple angiostatic proteins that could inhibit normal as well as abnormal blood vessels [43, 47, 48]. Current regulation systems could be combined to regulate multiple transgenes. However, due to the requirements of the systems, such an approach would be very cumbersome. The experiments described herein demonstrate the use of alternative splicing as a strategy to independently control the expression of more than four different transgenes in the same organism. Although AAV vectors are exemplified in these studies, this multi-regulation system could also employ other gene transfer vectors including lentivirus and other retroviruses, adenovirus, herpes virus, etc., as well as artificial chromosomes. Furthermore, this study addresses important public health problems, because it describes new treatments for established ocular neovascularization that occurs in age-related macular degeneration and diabetic retinopathy, the most common causes of severe vision loss in the US [48].

Kinetics of transgene expression for AAV vectors in an eye model. To study the kinetics of transgene expression in an eye model, AAV1-5 vectors carrying a GFP expression cassette were injected subretinally into wild-type Wistar rats and in vivo fluorescence imaging was performed to monitor transgene expression [17]. At 12 days post injection, GFP expression could be detected in AAV5, -4 and -1-injected animals, with type 5 and 4 vectors displaying the most intense GFP signal. No signal was detected in animals injected with AAV2 or -3 vectors at this concentration and time point. At 26 days post injection, GFP expression increased proportionally for AAV5, -4, and -1 vectors, with types 2 and 3 eventually displaying a small but positive signal. This trend continued for up to 46 days and these animals remained positive for the duration of the experiment (4 months). These data indicate that AAV5 could be the delivery system of choice for high levels of ocular transgene expression.

A self-complementary AAV (scAAV) for facilitating robust transgene expression at a minimal dose has also been developed [18]. This type of AAV vector contains a double stranded genome, thereby circumventing the rate-limiting step of second-strand synthesis for single-stranded AAV genomes. To study the kinetics of transgene expression mediated by scAAV in an eye model, a scAAV vector carrying a GFP expression cassette was injected subretinally into mice [63]. The standard single stranded AAV (ssAAV) vector carrying a GFP expression cassette was used for comparison. Injection of 5×10⁸ viral particles (vp) of scAAV vector resulted in GFP expression in almost all retinal pigment epithelial (RPE) cells within the area of the small detachment caused by the injection by three days and strong, diffuse expression by seven days. Expression was strong in all retinal cell layers by days 14 and 28. In contrast, three days after subretinal injection of 5×10⁸ vp of ssAAV vector, GFP expression was detectable in few RPE cells. Moreover, the ssAAV vector required 14 days for the attainment of expression levels comparable to those observed using scAAV at day 3. Expression in photoreceptors was not detectable until day 28. Dose-response experiments confirmed that onset of GFP expression was more rapid and robust after subretinal injection of scAAV vector than of ssAAV vector, resulting in pronounced expression in photoreceptors and other retinal neurons. Similar results were obtained for intravitreous injections. These data suggest that scAAV vectors may be advantageous for ocular gene therapy, particularly in retinal diseases that require rapid and robust transgene expression.

Suppression of ocular neovascularization by targeting survival factors supporting neovascularization. Studies have been undertaken to identify molecular signals that participate in ocular neovascularization in order to develop strategies for suppressing ocular neovascularization by targeting these molecular signals. Below is a list of factors and strategies studied. As indicated, blocking both VEGF and PDGF receptors simultaneously is one of the most efficacious treatments for ocular neovascularization in animal models.

1) Vascular endothelial growth factor (VEGF). VEGF is upregulated by hypoxia and is a major stimulatory factor for retinal neovascularization. In a murine model of choroidal neovascularization (CNV), mice were treated with small molecules of VEGF antagonist and it was found that the drugs caused strong suppression of the CNV [50, 51] or retina neovascularization [64]. Two patients with CNV caused by pathological myopia were treated with Bevacizumab, a recombinant humanized full length anti-VEGF monoclonal antibody that binds all isoforms of VEGF-A. Bevacizumab caused resorption of subretinal and intraretinal fluid, involution of subfoveal CNV, and improvement in visual acuity [65] confirming that VEGF is an important stimulus for CNV in pathologic myopia. In another study, ten patients with diabetic macular edema were treated with intraocular injections of Ranibizumab, an antibody fragment that binds all isoforms of VEGF-A. All patients showed improvement in to visual acuity and foveal thickness [66]. This indicates that VEGF is an important therapeutic target for diabetic macular edema.

Because there is a substantial increase in VEGF receptors in endothelial cells participating in CNV, studies were conducted to determine whether VEGF121, the most soluble isoform, can be used as a tool to direct destructive therapy to CNV [67]. After intravenous injection of 45 mg/kg of a chimeric protein consisting of VEGF121 coupled to the toxin gelonin (VEGF/rGel), but not uncoupled gelonin, there was immunofluorescent staining for gelonin within CNV in mice and regression of the CNV occurred. Intraocular injection of 5 ng of VEGF/rGel also caused significant regression of CNV or retinal neovascularization. Thus VEGF is both a target and a homing device for treatment of CNV. Placental growth factor (PIGF) is a VEGF family member that contributes to ocular neovascularization [68]. Clinical trials have been initiated to determine whether additional improvement can be achieved by inhibiting PIGF as well as all isoforms of VEGF165 by using VEGF Trap, a recombinant fusion protein consisting of the binding domains of VEGF receptors 1 and 2 and an Fc fragment of IgG that binds VEGF-A, PIGF, and other members of the VEGF family that bind VEGF receptors 1 or 2 [69]. Systemic administration of 1 mg/kg of VEGF Trap in patients with CNV due to age-related macular degeneration reduced leakage and retinal thickening, but 3 mg/kg caused substantial hypertension [70]. Intraocular injection of VEGF Trap is currently being tested to determine if there are advantages to more generalized blockade of VEGF family members compared to specific blockade of VEGF-A.

2) Platelet-derived growth factor (PDGF). Combining antagonism of PDGFs with blockade of VEGFs may be a useful strategy for treatment of ocular neovascularization. Increased expression of PDGF-B in the retina causes severe proliferative retinopathy and retinal detachment like the most advanced stages of proliferative diabetic retinopathy [71]. Endothelial cells produce PDGF-B, which promotes the recruitment, proliferation and survival of pericytes. PDGF-B also recruits glial cells and retinal pigmented epithelial (RPE) cells [72], which promotes scarring, a complication of ocular neovascularization that is the major cause of permanent loss of vision. Antagonists of PDGFs may help to reduce scarring, but may also synergize with VEGF antagonists to reduce neovascularization through their antagonism of pericytes, which provide survival signals for endothelial cells of new vessels [73]. Kinase inhibitors that block both VEGF and PDGF receptors are some of the most efficacious drugs for the treatment of ocular neovascularization in animal models [50, 51, 64].

3) Angiopoietin-2 (Ang2). Both angiopoietin-1 (Ang1) and -2 are binding partners of Tie2 receptor, which is selectively expressed on vascular endothelial cells and is required for embryonic vascular development [74, 75]. Ang1 binds with high affinity and initiates Tie2 phosphorylation and downstream signaling [76]. In contrast, Ang2 binds with high affinity, but does not stimulate phosphorylation of Tie2 in cultured endothelial cells [77]. In vitro, Ang2 acts as a competitive inhibitor of Ang1. It decreases Ang1 binding to Tie2 and Ang1-induced phosphorylation. In mice with ischemic retinopathy, induction of Ang2 at time points when ischemia (and VEGF) was less, hastened regression of neovascularization [78]. In triple transgenic mice that co-expressed VEGF and Ang2, the increased expression of Ang2 inhibited VEGF-induced neovascularization in the retina. Increased expression of Ang2 also resulted in regression of choroidal neovascularization. These studies indicate that ocular neovascularization is sensitive to Ang2 and that a high Ang2/VEGF ratio promotes regression of neovascularization.

4) Antiangiogenic peptides. There are several antiangiogenic peptides that may function under normal circumstances to limit and control neovascularization, but become overwhelmed in situations in which pathologic angiogenesis occurs. These peptides have been shown to inhibit retinal and/or CNV including the noncollagenous domain of a2(IV) [79], endostatin [80, 81], PEDF [82-84], and soluble VEGF receptor-1 (Flt-1) [85].

5) Vasohibin. Vasohibin differs from other inhibitors because it is up-regulated by VEGF and FGF 2 in cultured endothelial cells and therefore was hypothesized to function as a negative feedback regulator [86]. Studies testing this hypothesis in mice with ischemic retinopathy showed increased expression of VEGF was accompanied by elevation of vasohibin mRNA and blocking of the increase in VEGF mRNA with VEGF siRNA significantly attenuated the rise in vasohibin mRNA; knockdown of vasohibin increased the amount of neovascularization and over-expression of vasohibin reduced the amount of neovascularization [87]. Knockdown of vasohibin mRNA in ischemic retina had no significant effect on VEGF or VEGF receptor 1 mRNA levels, but caused a significant elevation in the level of VEGF receptor 2 mRNA. These data demonstrate that vasohibin acts as a negative feedback regulator of neovascularization in the retina, and indicate that suppression of VEGF receptor 2 may play some role in mediating its activity.

6) Stromal derived factor-1 (SDF-1). Circulating bone marrow-derived cells are likely to increase the levels and alter the gradients of angiogenic factors, thereby contributing to maladaptive, disorganized vessel growth. VEGF acting through VEGF receptor 1 recruits bone-marrow derived cells [88], but stromal derived factor-1 (SDF-1) acting through CXCR4 may also participate [89]. SDF-1 levels have been shown to be increased in ischemic retina and antagonists of CXCR4 suppress several types of ocular neovascularization [90].

7) Insulin-like growth factor-1 (IGF-1). There is substantial evidence suggesting that IGF-1 contributes to retinal neovascularization in proliferative diabetic retinopathy, although there is disagreement as to whether the contribution is major [91] or modest [92]. IGF-1 post-transcriptionally upregulates hypoxia-inducible factor-1 (HIF-1) [93], which not only increases VEGF, but also increases the products of other genes that contain a hypoxia response element (HRE) in their promoter, such as Angiopoietin 2 (Ang2). Increased expression of VEGF in the retina causes new vessels to sprout from the deep capillary bed, but not the superficial retinal vessels [94, 95], whereas co-expression of VEGF and Ang2 causes neovascularization that grows from the surface of the retina [96]. This explains why long-term expression of IGF-1 causes neovascularization that grows from the surface of the retina; it essentially mimics retinal hypoxia by up-regulating HIF-1.

As described herein, successful use of the inducible system of this invention in mouse eye has been demonstrated. These studies indicate that onset of reporter gene expression in retina and RPE is detectable within a day of LNA654 injection. However, the complete kinetics including the onset, duration and level of transgene expression will be studied under various conditions of induction. In the present system, induction of transgene expression depends on hybridization of ASO to the alternative splice site and subsequent correct splicing of the transgene message, therefore any factor effecting the uptake, intracellular transport, nuclear accumulation and degradation of a given ASO would determine the kinetics of transgene expression. In the eye, uptake of free oligonucleotides is relatively efficient [97-99]. Thus, to minimize the variables, studies will be conducted with free LNA654 to determine its induction of transgene expression. The kinetics will be studied by varying both the dose and the repetition of LNA654 administration. Experimental Design. Two reporter genes will be used: 1) firefly luciferase, for its advantage of easy and non-invasive monitoring of in vivo expression, including the eye [100, 101], as well as its broad-range and reliable quantification; and 2) green fluorescent protein (GFP) for its convenience of visualizing the transduced cells. Luciferase will be used as the first reporter gene for the following reasons: i) the luciferase assay is more sensitive, convenient, rapid and widely used; ii) the detection range is extremely broad; iii) no endogenous background activity has been reported in transgenic animals or gene transfer studies; and iv) luciferase protein has a short half life of about 2 h [102, 103]. Therefore, luciferase is a much more practical marker gene for studying the kinetics of transgene expression. The GFP marker gene will be subsequently used for cell type identification, and if needed for kinetic studies. In order to limit promoter bias, the ubiquitous CB promoter will be used. To deliver the transgene expression cassette, the AAV5 vector will be used, as it has been shown to mediate the most robust transgene expression in the eye. Therefore, the pAAV654Luc and pAAV654GFP plasmids will be used to generate their corresponding AAV5 vectors for the proposed kinetic studies. These two vectors successfully mediated regulated transgene expression both in vitro and in vivo.

In the first set of experiments to study the onset, duration and level of transgene expression, 1×10⁹ genomic particles of AAV654Luc vector will be delivered to each mouse eye via intravitreous injection (n=55). At day 46 post vector administration, the treated mice will be injected intravitreously with ASO to induce luciferase expression. Day 46 is selected because at this time the conversion of the single-stranded AAV genome to its double-stranded form is mostly complete as shown in previous studies. A single dose of LNA654 or the control LNA654M will be used at 0, 0.1, 0.3, 1, 3, 10 ug in 1 ul (n=5 for each ASO dose). In previous studies, it was shown that 0.556 ug of LNA654 induced detectable GFP expression. The last dose of ASO is based on the highest concentration typically prepared. At days 0, 1, 3, 7, 14, 21, 28, 35 and 42 post ASO injection, the mice will be imaged to quantify the level of luciferase expression. If necessary, the dose of ASO and the schedule of luciferase imaging will be adjusted, pending the outcome of the experiment. Results collected will be used to plot a kinetic graph of time vs. expression level for each dose.

In a second set of experiments, a determination will be made of whether repeated dosing of ASO will modify the kinetics of transgene expression. Repeated dosing of LNA654 was shown in previous studies to increase the expression of AAV654Luc in mouse liver. The design for this set of experiments is similar to that for the first set as described above, except that only the single optimal dose determined will be used for repeated dosing. Two repeated dosings will be administered at days 1 and 3 (n=5 for LNA654 or LNA654M) as well as three repeated dosings at days 1, 3 and 5 (n=5 for LNA654 or LNA654M). Data obtained will be compared to those from the first set of experiments.

In a third set of experiments, a determination will be made of whether the duration of induced transgene expression can be prolonged by additional LNA654 administration. The design for this set of experiments is similar to and will be based on the outcomes of the first and second sets. The optimal dose and repetition to prolong the level of transgene expression (n=5 for LNA654 or LNA654M) will be determined. The onset and duration of transgene expression as demonstrated in the aforementioned experiments will assist in a determination of the timing of additional LNA654 administration. These results will be compared with those from the first and second sets of experiments.

In a fourth set of experiments, studies will be conducted to identify the cell types responsible for the transgene expression and their relative levels of transgene expression. To do this, 1×10⁹ genomic particles of AAV654GFP vector will be delivered to each mouse eye via intravitreous injection (n=20). At day 46 post vector administration, half of the treated mice (n=10) will be injected intravitreously with an optimal dose of LNA654 or the control LNA654M with an optimal repetition as determined in the aforementioned experiments. At the time point corresponding to the peak level of luciferase expression, the mice will be euthanized and half of the eyes in each group (n=5) will be removed to make flat mounts or cryosections. The flat mounts and cryosections will be examined by fluorescence microscopy to examine the cell types and their relative levels of GFP expression.

AAV vector preparation. AAV vectors will be generated, purified and titered as described [104]. 293 cells in 15-cm plates are transfected using the polyethylenimine (PEI) transfection method with a mixture of three plasmids consisting of an AAV vector plasmid, an AAV helper plasmid XX2, and an adenovirus helper plasmid XX6-80. Forty-eight to 72 hours post-transfection, the cells are harvested for isolation of nuclei. The nuclei are then sonicated to release AAV virions and digested with DNase to facilitate the purification of AAV. The resulting extract is subjected to two consecutive steps of cesium chloride gradient ultracentrifugation. AAV from the gradient is fractionated and dialyzed against PBS. The titer of the virus produced is determined by using a dot blot assay.

Intraocular delivery of AAV vector and ASO. Mice will be treated humanely in strict compliance with the Association for Research in Vision and Ophthalmology statement on the use of animals in research. Four week old Balb/c mice will be given an intravitreous or subretinal injection of 1 μl containing 10⁹ genome particles of AAV vector with a Harvard pump apparatus and pulled glass micropipettes as previously described [82]. Micropipettes are calibrated to deliver 1 μl of vehicle upon depression of a foot switch. For intravitreous injections, the adult female Balb/c mice are anesthetized, and under a dissecting microscope, the sharpened tip of the micropipette is passed through the sclera just behind the limbus into the vitreous cavity and the foot switch is depressed. Subretinal injections are performed using a condensing lens system on the dissecting microscope, with a plastic ring filled with Gonioscopic solution (Alcon, Fort Worth, Tex.), which allows visualization of the retina during the injection. The pipette tip is passed through the sclera posterior to the limbus and is positioned just above the retina. Depression of the foot switch causes a jet of injection fluid to penetrate the retina. This technique is very atraumatic and the direct visualization allows confirmation that the injection is successful, because of the appearance of a small retinal detachment (bleb). At day 46 after injection of vector, mice will be given an intravitreous injection of 1 μl containing LNA654 or LNA654M and at various time points afterwards, transgene expression will be determined.

In vivo luciferase imaging. Mice will be anesthetized by intraperitoneal (i.p.) injection of 2.5% Avertin (0.4 mg/g body weight). Luciferin (125 ul at 25 mg/ml) will then be injected i.p. into each mouse to allow in vivo assay of firefly luciferase activity. The mice will be imaged using the IVIS imaging system (Xenogen).

Flat mounts and cryosections. Mice will be euthanized and the eyes will be removed and fixed with 4% paraformaldehyde in PBS for 1 hour and with 10% phosphate-buffered formalin overnight to make flat mounts [63]. The cornea and lens are removed and the entire retina is carefully dissected from the eyecup. Radial cuts are made from the edge to the equator of the retina and the retina is flat mounted in Aquamount mounting medium with the photoreceptor facing down. Radial cuts are also made in eyecups and they are flat mounted with the sclera facing down (choroidal flat mounts). For cryosections, eyes are fixed in 4% paraformaldehyde and 5% sucrose in PBS for 1 hour and are washed with 20% sucrose in PBS overnight. Eyes are then embedded in optimal cutting temperature embedding compound (OCT; Miles Diagnostics, Elkhart, Ind.). Ocular frozen sections are rinsed in PBS and mounted with Aquamount mounting medium. Flat-mounts and sections are examined by fluorescence microscopy (Axioskop microscope; Zeiss, Thornwood, N.Y.), and images are digitized using a three-color charge-coupled device (CCD) video camera (IK-TU40A; Toshiba, Tokyo, Japan) and a frame grabber.

Previous studies have successfully demonstrated induction of luciferase expression in both liver and heart mediated by AAV654LucB. Successful induction of GFP expression in mouse eye has also been demonstrated. Although the basal level of luciferase expression in both liver and heart was acceptable, 10.4 fold lower than the peak level, it is possible that in the eye the basal level for AAV654LucB could be higher, similar to that in 293 cells in vitro. If this should be the case and the higher basal level would interfere with the kinetic study, the AAV vector derived from construct A(S0)-B(S0) or B(S0−CT) will be used instead of AAV654LucB. Constructs A(S0)-B(S0) and B(S0−CT) had induction levels of 120 and 24 fold, respectively (FIG. 7). Another potential problem with respect to expressing luciferase in mouse eye is that an immune response could theoretically be elicited against the exogenous luciferase protein, although there have been no such reports in the literature to the inventors' knowledge. Long-term expression of luciferase in other major organs in mice has been successfully demonstrated, suggesting that the protein is not efficiently presented to the immune system [105-107]. However, in the event of immune response developed in the eye, immune deficient nude mice will be used for the kinetic studies. Alternatively, GFP will be used as a reporter gene since its expression in the eye of immune competent mouse has been reported to sustain at least 11 weeks [108]. GFP has been used as a marker gene to compare the kinetics of transgene expression mediated by conventional single-strand and self-complementary AAV vectors [63]. For kinetic studies, the original GFP protein could be too stable and use of the destabilized green fluorescent protein may be more preferable [109]. The expression of the latter protein could be induced at least twice over a period of 6 months [110].

Ocular neovascularization and gene therapy. Ocular neovascularization is a complex multi-step process that is controlled by a number of factors [47, 48]. Therapeutic manipulation of a combination of these factors offers the potential means to effectively control ocular neovascularization. Such a strategy for controlling neovascularization is likely to require the long-term presence of multiple angiostatic proteins in the eye. However, if the expression of multiple angiostatic proteins is unregulated, then a possibility of a risk of adverse local effects could be created. Thus, the development of strategies to enable appropriate regulation of multiple transgene expression is desirable to minimize the potential for local toxicity. To validate the inducible system of this invention for differentially regulating the expression of multiple transgenes in the eye, the studies described herein using marker genes will be extended in vitro to normal eyes. Following the validation, studies will be conducted to test the inducible system for the ability to regulate the expression of multiple potent blockers for survival factors supporting neovascularization in an ocular neovascularization model. These potent factors have different mechanisms of action and may have synergistic effects in suppressing and regressing neovascularization. The effect of regulating both the level and the duration of expression of these blockers in treating neovascularization will be analyzed.

Experimental Design. To facilitate the validation in the eye of this inducible system for differentially regulating the expression of multiple transgenes, the same panel of fluorescent protein expression cassettes will be used, which includes green, red and blue fluorescent protein genes, each inserted with a different alternative splicing intron. The constructs will be separately packaged into AAV (e.g., AAV5) vectors for efficient gene transfer in vivo. The resulting vectors will then be mixed and injected into mouse eyes at 1×10⁹ genomic particles per eye (n=20). At day 46 post vector administration, the treated mice will be injected intravitreously with each of the three corresponding ASOs to induce transgene expression (n=5 per ASO). Another group will receive a control ASO (n=5). The optimal dose of ASO will be used, as determined according to the teachings set forth herein. At the time point corresponding to the peak level of luciferase expression, the mice will be euthanized and the eyes will be removed to make flat mounts. The flat mounts will be examined by fluorescence microscopy to determine the differential regulation of expression of multiple transgenes. Each ASO is expected to induce the specific expression of the fluorescent protein gene that is regulated by its corresponding alternative splicing intron.

Following the validation, the inducible system will be tested for the ability to regulate the expression of multiple potent blockers for survival factors supporting neovascularization in an ocular neovascularization model. Previous studies using small molecules of antagonists and transgenic mice showed that blocking both VEGF and PDGF receptors as well as increasing Ang2/VEGF ratio promoted regression of neovascularization and therefore the following three therapeutic genes are expected to have synergistic effects in suppressing neovascularization and will be used in this study: i) VEGF Trap, a fusion gene consisting of the binding domains of VEGF receptors 1 and 2 and an Fc fragment of IgG. VEGF Trap is intended to antagonize the actions of VEGF-A, PIGF, and other members of the VEGF family that bind VEGF receptors 1 or 2; ii) PDGFtrap, a fusion gene consisting of soluble PDGF receptor-β (sPDGFRβ) and IgG1-Fc, intended to antagonize the action of PDGFs; and iii) Ang2, a binding partner of Tie2 without stimulating phosphorylation of the receptor. Ang2 is intended to cause a blockade of Tie receptors.

To differentially regulate the expression of the aforementioned potent blockers, a different alternative splicing intron will be inserted into each of the transgenes. The regulation of expression of the resulting constructs by their corresponding ASOs will be confirmed in vitro by transfection and subsequent analysis of the splicing pattern of the transgene mRNA. The resulting constructs will be packaged into AAV5 vectors and the effect of regulating both the level and the duration of expression for each of these blockers alone in treating neovascularization will be studied. Specifically, for each of the vectors, 1×10⁹ genomic particles will be injected into each eye (n=20). At day 39 post vector administration, the mice will receive laser-induced rupture of Bruch's membrane in three locations in each vector injected eye. At 1 week after laser, five of the treated mice will be perfused with fluorescein-labeled dextran and the baseline amount of CNV at one week will be measured. On the same day, i.e., day 46 post vector administration, mice in the experimental (n=10) and control (n=5) groups will be injected intravitreously with a specific ASO and a control ASO, respectively. The optimal does and repetition will be used as described herein. At a time point corresponding to the initial decrease of luciferase expression, five mice each from the experimental and the control group will be perfused with fluorescein-labeled dextran and the area of CNV at each rupture site will be measured by image analysis on choroidal flat mounts. ELISA will be used to measure the levels of transgene expression in the eyes of the other experimental group (n=5). If transgene expression causes substantial regression of CNV, the experiments will be repeated, changing the waiting time between laser and the ASO administration from 1 week to 1 month. If there is substantial regression of 1-month old CNV, then 3-month old CNV will be tested. This will allow a determination to be made of whether CNV matures over time and becomes less resistant to the expression of the therapeutic genes.

The combination effect of the transgenes in treating neovascularization will also be tested. The combinations of VEGFtrap/PDGFtrap, VEGFtrap/Ang2 and VEGFtrap/PDGFtrap/Ang2 will be evaluated. The design of the combination study will be essentially the same as that for the single transgene described above except that mixtures of AAV vectors as well as ASOs will be used to mediate expression of the multiple transgenes. If the combination therapy causes substantial regression of CNV, the amount and repetition of each ASO will be decreased stepwise to determine the optimal level and duration of expression for each of the transgenes.

Regulated expression of fluorescent protein genes in the eye. AAV preparation, ocular injection, examinations and insertion of introns into therapeutic transgenes will be carried out as described herein. To analyze the splicing pattern of transgene mRNA, 293 cells in each 24-well plate will be transfected with 50 ng of the appropriate plasmid and 8.3 pmole of the appropriate ASO using the calcium phosphate transfection method. At 24 hours after transfection, the treated cells will be harvested for RNA isolation using the RNeasy Mini Kit (Qiagen, cat #74104). The splicing pattern of the transgene mRNA will be analyzed by using an RT-PCR assay and electrophoresis on an 8% polyacrylamide gel. Specifically, total RNA isolated will be used as a template for an RT-PCR assay using primers to amplify the region of sequences encompassing the inserted intron. Thus, the sizes of the RT-PCR products would reflect the splicing pattern of the transgene mRNA.

Mouse model of CNV. The model of CNV due to laser-induced rupture of Bruch's membrane to mice [114] has been adapted and used to explore the role of various stimulators and therapeutic agents [51, 80-82, 115-117]. Investigations in this model showed that VEGF antagonists are good inhibitors of CNV [51, 116], and subsequent clinical trials have shown that the model is predictive for effects in human disease. By allowing the CNV to develop prior to instituting treatment, it has been possible to identify treatments that result in regression of established neovascularization [67, 78, 83, 118, 119]. In mice, as opposed to what has been reported in monkeys, CNV does not regress spontaneously for at least six months after rupture of Bruch's membrane, the longest time point examined.

Adult mice are anesthetized with ketamine hydrochloride (100 mg/kg body weight), pupils are dilated with 1% tropicamide, and diode laser photocoagulation is used to rupture Bruch's membrane at three locations in each eye. Laser photocoagulation (532 nm wavelength, 100 μm spot size, 0.1 seconds duration, and 120 mW intensity) is delivered using the slit lamp delivery system and a hand-held cover slide as a contact lens. Burns are performed in the 9, 12, and 3 o'clock positions 2-3 disc diameters from the optic nerve. Production of a vaporization bubble at the time of laser, which indicates rupture of Bruch's membrane, is an important factor in obtaining CNV [114], so only burns in which a bubble is produced are included in the analyses. At various times after rupture of Bruch's membrane, a cohort of mice is used to measure the baseline amount of CNV at that time point. Treatment is then instituted and after 1 or 4 weeks of treatment, the amount of CNV is measured in treated and control mice.

Measurement of the area of CNV at rupture sites. The area of CNV at each rupture site is measured in choroidal flat mounts after perfusion with fluorescein-labeled dextran [120]. Mice are anesthetized and perfused with 1 ml of phosphate-buffered saline containing 50 mg/ml of fluorescein-labeled dextran (2×10⁶ average mw, Sigma, St. Louis, Mo.). The eyes are removed and fixed for 1 hour in 10% phosphate-buffered formalin. The to cornea and lens are removed and the entire retina is carefully dissected from the eyecup. Radial cuts are made from the edge to the equator and the eyecup is flat mounted in Aquamount with the sclera facing down. Flat mounts are examined by fluorescence microscopy on an Axioskop microscope (Zeiss, Thornwood, N.Y.) and images are digitized using a 3 CCD color video camera (IK-TU40A, Toshiba, Tokyo, Japan) and a frame grabber. Image-Pro Plus software (Media Cybernetics, Silver Spring, Md.) is used to measure the total area of hyperfluorescence associated with each burn, corresponding to the total fibrovascular scar.

Enzyme-linked immunoabsorbant assay (ELISA). ELISAs will be done as described [95]. Eyes are removed, a cut is made at the limbus, the lens is carefully removed, and the remainder of the eye is snap-frozen in liquid nitrogen. Eye homogenates are prepared by dounce homogenization followed by three freeze/thaw cycles in phosphate-buffered saline with 100 μM PMSF. Homogenates are microfuged and the protein concentration of supernatants is measured using a BioRad Protein Assay Kit (BioRad, Hercules, Calif.). ELISA of the samples will be performed using the appropriate kits. Serial dilutions of purified proteins will be used to generate standard curves.

Statistical analysis. Statistical comparisons are done using a linear mixed model [121]. This model is analogous to analysis of variance (ANOVA), but allows analysis of all CNV area measurements from each mouse rather than average CNV area per mouse by accounting for correlation between measurements from the same mouse. The advantage of this model over ANOVA is that it accounts for differing precision in mouse-specific average measurements arising from a varying number of observations among mice. In some instances, a log transformation is used on the area measurements prior to analysis so that they better meet the normal distribution assumption of the analytic model. P-values for comparisons of treatments are adjusted for multiple comparisons using Dunnett's method.

The foregoing examples are illustrative of the present invention, and are not to be construed as limiting thereof. The invention is described by the following claims, with equivalents of the claims to be included therein.

All publications, patent applications, patents, patent publications, GenBank® database sequences and other references cited herein are incorporated by reference in their entireties for the teachings relevant to the sentence and/or paragraph in which the reference is presented.

TABLE 1
Primers used in RT-PCR assays

| Primer | | Target | Size of PCR | |
|---|---|---|---|---|
| Pair | Sequences | Gene | Product (bp) | SEQ ID |
| GFPf | 5′-CGTAAACGGCCACAAGTTCAGCG-3′ | GFP | 160 (AS) | 362 |
| GFPr | 5′-GTGGTGCAGATGAACTTCAGGGTC-3′ | | 87 (CS) | 363 |
| Af | 5′-GCCTACCGTGGTGTTCGTTTCC-3′ | Luciferase | 202 (AS) | 364 |
| Ar | 5′-GTACATCGACTGAAATCCCTGGTAATCCG-3′ | | 129 (CS) | 365 |
| Bf | 5′-ATCTACCTCCCGGTTTTAATGAATACGATTTTGTGCCAGA-3′ | Luciferase | 369 (AS) | 366 |
| Br | 5′-CTTCAAATCTATACATTAAGACGACTCGAAATCCACA-3′ | | 296 (CS) | 367 |
| Cf | 5′-CTCCTTCTTCGCCAAAAGCACTCTGATTG-3′ | Luciferase | 380 (AS) | 368 |
| Cr | 5′-GGACCTCTCACACACAGTTCGCC-3′ | | 307 (CS) | 369 |
| Df | 5′-GGCGAACTGTGTGTGAGAGGTCC-3′ | Luciferase | 480 (AS) | 370 |
| Dr | 5′-CGGTACTTCGTCCACAAACACAACTCC-3′ | | 407 (CS) | 371 |
| Ff | 5′-GCTATTCCAGAAGTAGTGAGGAGGCTTTTTTGG-3′ | Luciferase | 435 (AS) | 372 |
| Fr | 5′-CACCGGCATAAAGAATTGAAGAGAGTTTTCACTGC-3′ | | 362 (CS) | 373 |
| AATf | 5′-CCGTGAAGGTGCCTATGATGAAGCGTTTAGTC-3′ | AAT | 203 (AS) | 374 |
| AATr | 5′-CCCCTCATCAGGCAGGAAGAAGATGGCGGT-3′ | | 130 (CS) | 375 |

TABLE 2

| | ASO Target Sequences | SEQ ID NO: |
|---|---|---|
| Set 1 | T g G g T T A a g g t a a t a G | 378 |
| | A g C g A A T a g g t a a t a C | 379 |
| | C g A g G G C a g g t a a t a A | 380 |
| | G g T g C C G a g g t a a t a T | 381 |
| Set 2 | T g G g T T A a g g t a a t a G | 382 |
| | G g T g A C T a g g t a a t a C | 383 |
| | C g A g G G C a g g t a a t a T | 384 |
| | A g C g C A G a g g t a a t a A | 385 |

Capital letters indicate positions at which mutations are introduced. The top sequences of Set 1 and 2 are the original ASO target in the SO-CT (also the IVS2-654) intron.

TABLE 3

| | | | | | | | | | Induction |
|---|---|---|---|---|---|---|---|---|---|
| | −2 | −1 | 1 | 2 | 3 | 4 | 5 | 6 | Level |
| Consensus | A | G | [symbol] | [symbol] | A/G | A | G | T | |
| IVS2-654 | A | G | [symbol] | [symbol] | A | A | T | A | 4.3 |
| 658T | A | G | [symbol] | [symbol] | A | A | T | T | 7.3 |
| 657G | A | G | [symbol] | [symbol] | A | A | G | A | 9.2 |
| 657GT | A | G | [symbol] | [symbol] | A | A | G | T | 223 |
| M3 | A | G | [symbol] | [symbol] | G | A | G | T | 164 |
| M6 | A | G | [symbol] | [symbol] | A | A | G | G | 5.7 |

TABLE 4

| | |
|---|---|
| Luciferase Activity | |
| (×10³ RLU) | Induction |

| Construct | +LAN654M | +LAN654 | Level (fold) |
|---|---|---|---|
| A(S0) | 24.5 ± 1.4 | 108.0 ± 7.2 | 4.4 |
| B(S0) | 7.9 ± 0.2 | 33.8 ± 2.3 | 4.3 |
| B(S0-GT) | 0.04 ± 0.01 | 9.9 ± 0.3 | 228 |
| B(S0-CT) | 2.0 ± 0.2 | 47.8 ± 0.8 | 24 |
| A(S0)-B(S0) | 0.6 ± 0.1 | 73.3 ± 2.0 | 120 |
| A | 36.1 ± 1.1 | 172.3 ± 10.0 | 4.8 |
| B | 23.5 ± 1.0 | 103.2 ± 7.3 | 4.4 |

TABLE 5

| | Luciferase Activity (RLU) | | | |
|---|---|---|---|---|
| | | | | +LNA654 |
| Plasmid | No ASO | + LNA654 | + LNAS1 | +LNAS1 |
| pGFP654 | 100 ± 0 | 93 ± 15 | 103 ± 12 | 106 ± 15 |
| pLucS1 | 343 ± 47 | 360 ± 36 | 120,066 ± 6,854 | 109,747 ± 2,435 |
| (pGFP654 + | 900 ± 101 | 1,213 ± 57 | 245,820 ± 25,482 | 224,727 ± 8,024 |
| pLucS1) | | | | |

What is claimed is:

1. A cell comprising a first heterologous nucleic acid construct and a second heterologous nucleic acid construct, wherein each of said first and second constructs comprises:
A. a first nucleotide sequence encoding a nucleotide sequence of interest (NOI), wherein the NOI is heterologous to the cell; and
B. a second intronic nucleotide sequence operably associated with said first nucleotide sequence, wherein said second intronic nucleotide sequence is heterologous to said first nucleotide sequence and heterologous to said cell, and further wherein said second intronic nucleotide sequence comprises:
i) a first intron defined by a first set of splice elements that is removed by splicing to produce a first RNA molecule encoded by said first nucleotide sequence, under conditions whereby removal of a second intron defined by a second set of splice elements is prevented; and
ii) said second intron defined by said second set of splice elements, wherein said second intron is different from said first intron, and wherein under conditions whereby removal of said second intron is not prevented and said second intron is removed by splicing, no RNA molecule and/or a second RNA molecule that is not encoded by said first nucleotide sequence is produced,
further wherein said NOI of said first nucleotide sequence of each of said first and second constructs is different from one another and wherein said second intronic nucleotide sequence of each of said first and second heterologous nucleic acid constructs is different from one another.

2. The cell of claim 1, wherein said first and second heterologous nucleic acid constructs are present in said cell as separate nucleic acid constructs.

3. The cell of claim 1, wherein said first and second heterologous nucleic acid constructs are present in said cell as a single nucleic acid construct.

4. The cell of claim 1, wherein said first nucleotide sequence encodes a nucleotide sequence of interest (NOI) selected from the group consisting of:
a) a nucleotide sequence encoding a protein or peptide;
b) a nucleotide sequence encoding a product having activity as an interfering RNA;
c) a nucleotide sequence encoding a product having enzymatic activity as an RNA;
d) a nucleotide sequence encoding a ribozyme;
e) a nucleotide sequence encoding an antisense sequence;
f) a nucleotide sequence encoding a small nuclear RNA (snRNA); and
g) any combination of (a)-(f) above

5. The cell of claim 1, wherein said second intronic nucleotide sequence of at least one of said first or second heterologous nucleic acid constructs is selected from the group consisting of:
a1) the nucleotide sequence of SEQ ID NO:92 (S0 257 by intron);
b1) the nucleotide sequence of SEQ ID NO:2 (S0−GT);
c1) the nucleotide sequence of SEQ ID NO:1 (S0−CT);
d1) the nucleotide sequence of SEQ ID NO:4 (S0−GT+CT);
e1) the nucleotide sequence of SEQ ID NO:3 (51);
f1) the nucleotide sequence of SEQ ID NO:5 (S1+CT);
g1) the nucleotide sequence of SEQ ID NO:6 (M3);
h1) the nucleotide sequence of SEQ ID NO:7 (M3+CT);
i1) the nucleotide sequence of SEQ ID NO:8 (M6);
j1) the nucleotide sequence of SEQ ID NO:9 (M6+CT);
k1) the nucleotide sequence of SEQ ID NO:14 (M3+S1);
l1) the nucleotide sequence of SEQ ID NO:16 (M3+S1+CT);
m1) the nucleotide sequence of SEQ ID NO:15 (M6+S1);
n1) the nucleotide sequence of SEQ ID NO:17 (M6+S1+CT);
o1) the nucleotide sequence of SEQ ID NO:22, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;
p1) the nucleotide sequence of SEQ ID NO:23, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;
q1) the nucleotide sequence of SEQ ID NO:24, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;
r1) the nucleotide sequence of SEQ ID NO:25, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;
s1) the nucleotide sequence of SEQ ID NO:26, which comprises the sequence X₁GX₃GX₅X₆X₇AGGTAAGTX₁₄ (SEQ ID NO:326), wherein X of any of X₁, X₃, X₅-X₇ and X₁₄ can be A, C, T or G, in any combination;
t1) the nucleotide sequence of SEQ ID NO:27, which comprises the sequence X₁GX₃GX₆X₆X₇AGGTAAGTX₁₄ (SEQ ID NO:326), wherein X of any of X₁, X₃, X₅-X₇ and X₁₄ can be A, C, T or G, in any combination;
u1) the nucleotide sequence of SEQ ID NO:28, which comprises the sequence X₁X₂X₃X₄X₆X₆AAGGTAATAG (SEQ ID NO:327), wherein X of any of X₁,-X₆ can be A, C, T or G, in any combination;
v1) the nucleotide sequence of SEQ ID NO:29, which comprises the sequence X₁X₂X₃X₄X₆X₆AAGGTAATAG (SEQ ID NO:327), wherein X of any of X₁,-X₆ can be A, C, T or G, in any combination;
w1) the nucleotide sequence of SEQ ID NO:30, which comprises the sequence X₁GX₃X₄X₆X₆X₇AGGTAATAG (SEQ ID NO:328), wherein X of any of X₁ and X₃-X₇ can be A, C, T or G, in any combination;
x1) the nucleotide sequence of SEQ ID NO:31, which comprises the sequence X₁GX₃X₄X₆X₆X₇AGGTAATAG (SEQ ID NO:328), wherein X of any of X₁, and X₃-X₇ can be A, C, T or G, in any combination;
y1) the nucleotide sequence of SEQ ID NO:32, which comprises the sequence X₁X₂GX₄X₆X₆X₇AGGTAATAG (SEQ ID NO:329), wherein X of any of X₁, X₂ and X₄-X₇ can be A, C, T or G, in any combination;
z1) the nucleotide sequence of SEQ ID NO:33, which comprises the sequence X₁X₂GX₄X₆X₆X₇AGGTAATAG (SEQ ID NO:329), wherein X of any of X₁, X₂ and X₄-X₇ can be A, C, T or G, in any combination;
a2) the nucleotide sequence of SEQ ID NO:34, which comprises the sequence X₁X₂X₃GX₆X₆X₇AGGTAATAG (SEQ ID NO:330), wherein X of any of X₁-X₃ and X₅-X₇ can be A, C, T or G, in any combination;
b2) the nucleotide sequence of SEQ ID NO:35, which comprises the sequence X₁X₂X₃GX₆X₆X₇AGGTAATAG (SEQ ID NO:330), wherein X of any of X₁-X₃ and X₅-X₇ can be A, C, T or G, in any combination;
c2) the nucleotide sequence of SEQ ID NO:36, which comprises the sequence X₁X₂X₃X₄TX₆X₇AGGTAATAG (SEQ ID NO:331), wherein X of any of X₁-X₄ and X₆-X₇ can be A, C, T or G, in any combination;
d2) the nucleotide sequence of SEQ ID NO:37, which comprises the sequence X₁X₂X₃X₄TX₆X₇AGGTAATAG (SEQ ID NO:331), wherein X of any of X₁-X₄ and X₆-X₇ can be A, C, T or G, in any combination;
e2) the nucleotide sequence of SEQ ID NO:38, which comprises the sequence X_{I}GGX₄X₆X₆X₇X₈GGTAATAG (SEQ ID NO:332), wherein X of any of X₁ and X₄-X₈ can be A, C, T or G, in any combination;
f2) the nucleotide sequence of SEQ ID NO:39, which comprises the sequence X₁GGX₄X₅X₈X₇X₈GGTAATAG (SEQ ID NO:332), wherein X of any of X₁ and X₄-X₈ can be A, C, T or G, in any combination;
g2) the nucleotide sequence of SEQ ID NO:40, which comprises the sequence X₁X₂GGX₅X₈X₇X₈GGTAATAG (SEQ ID NO:333), wherein X of any of X₁, X₂ and X₅-X₆ can be A, C, T or G, in any combination;
h2) the nucleotide sequence of SEQ ID NO:41, which comprises the sequence X₁X₂GGX₅X₈X₇X₈GGTAATAG (SEQ ID NO:333), wherein X of any of X₁, X₂ and X₅-X₈ can be A, C, T or G, in any combination;
i2) the nucleotide sequence of SEQ ID NO:42, which comprises the sequence X₁X₂X₃GTX₈X₇X₈GGTAATAG (SEQ ID NO:334), wherein X of any of X₁-X₃ and X₆-X₈ can be A, C, T or G, in any combination;
j2) the nucleotide sequence of SEQ ID NO:43, which comprises the sequence X₁X₂X₃GTX₈X₇X₈GGTAATAG (SEQ ID NO:334), wherein X of any of X₁-X₃ and X₆-X₈ can be A, C, T or G, in any combination;
k2) the nucleotide sequence of SEQ ID NO:44, which comprises the sequence X₁X₂X₃X₄TTX₇X₈GGTAATAG (SEQ ID NO:335), wherein X of any of X₁-X₄ and X₇-X₈ can be A, C, T or G, in any combination;
l2) the nucleotide sequence of SEQ ID NO:45, which comprises the sequence X₁X₂X₃X₄TTX₇X₈GGTAATAG (SEQ ID NO:335), wherein X of any of X₁-X₄ and X₇-X₈ can be A, C, T or G, in any combination;
m2) the nucleotide sequence of SEQ ID NO:46, which comprises the sequence X₁X₂X₃X₄X₅TAX₈GGTAATAG (SEQ ID NO:336), wherein X of any of X₁-X₅ and X₈ can be A, C, T or G, in any combination;
n2) the nucleotide sequence of SEQ ID NO:47, which comprises the sequence X₁X₂X₃X₄X₅TAX₈GGTAATAG (SEQ ID NO:336), wherein X of any of X₁-X₅ and X₈ can be A, C, T or G, in any combination;
o2) the nucleotide sequence of SEQ ID NO:48, which comprises the sequence X₁X₂X₃X₄X₅X₈AAGGTAAGTG (SEQ ID NO:337), wherein X of any of X₁-X₆ can be A, C, T or G, in any combination;
p2) the nucleotide sequence of SEQ ID NO:49, which comprises the sequence X₁X₂X₃X₄X₅X₈AAGGTAAGTG (SEQ ID NO:337), wherein X of any of X₁-X₆ can be A, C, T or G, in any combination;
q2) the nucleotide sequence of SEQ ID NO:50, which comprises the sequence X₁GX₃X₄X₅X₆X₇AGGTAAGTG (SEQ ID NO:338), wherein X of any of X₁ and X₃-X₇ can be A, C, T or G, in any combination;
r2) the nucleotide sequence of SEQ ID NO:51, which comprises the sequence X₁GX₃X₄X₅X₈X₇AGGTAAGTG (SEQ ID NO:338), wherein X of any of X₁ and X₃-X₇ can be A, C, T or G, in any combination;
s2) the nucleotide sequence of SEQ ID NO:52, in any combination, which comprises the sequence X₁X₂GX₄X₅X₈X₇AGGTAAGTG (SEQ ID NO:339), wherein X of any of X₁-X₂ and X₄-X₇ can be A, C, T or G;
t2) the nucleotide sequence of SEQ ID NO:53, which comprises the sequence X₁X₂GX₄X₅X₈X₇AGGTAAGTG (SEQ ID NO:339), wherein X of any of X₁-X₂ and X₄-X₇ can be A, C, T or G, in any combination;
u2) the nucleotide sequence of SEQ ID NO:54, which comprises the sequence X₁X₂X₃GX₅X₆X₇AGGTAAGTG (SEQ ID NO:340), wherein X of any of X₁-X₃ and X₅-X₇ can be A, C, T or G, in any combination;
v2) the nucleotide sequence of SEQ ID NO:55, which comprises the sequence X₁X₂X₃GX₅X₆X₇AGGTAAGTG (SEQ ID NO:340), wherein X of any of X₁-X₃ and X₅-X₇ can be A, C, T or G, in any combination;
w2) the nucleotide sequence of SEQ ID NO:56, which comprises the sequence X₁X₂X₃X₄TX₆X₇AGGTAAGTG (SEQ ID NO:341), wherein X of any of X₁-X₄ and X₆-X₇ can be A, C, T or G, in any combination;
x2) the nucleotide sequence of SEQ ID NO:57, which comprises the sequence X₁X₂X₃X₄TX₈X₇AGGTAAGTG (SEQ ID NO:341), wherein X of any of X₁-X₁and X₆-X₇ can be A, C, T or G, in any combination;
y2) the nucleotide sequence of SEQ ID NO:58, which comprises the sequence X₁GGX₄X₅X₆X₇X₈GGTAAGTG (SEQ ID NO:342), wherein X of any of X₁ and X₄-X₈ can be A, C, T or G, in any combination;
z2) the nucleotide sequence of SEQ ID NO:59, which comprises the sequence X₁GGX₄X₅X₆X₇X₈GGTAAGTG (SEQ ID NO:342), wherein X of any of X₁ and X₄-X₈ can be A, C, T or G, in any combination;
a3) the nucleotide sequence of SEQ ID NO:60, which comprises the sequence X₁X₂GGX₅X₈X₇X₈GGTAAGTG (SEQ ID NO:343), wherein X of any of X₁-X₂ and X₅-X₈ can be A, C, T or G, in any combination;
b3) the nucleotide sequence of SEQ ID NO:61, which comprises the sequence X₁X₂GGX₅X₈X₇X₈GGTAAGTG (SEQ ID NO:343), wherein X of any of X₁-X₂ and X₅-X₈ can be A, C, T or G, in any combination;
c3) the nucleotide sequence of SEQ ID NO:62, which comprises the sequence X₁X₂X₃GTX₈X₇X₈GGTAAGTG (SEQ ID NO:344), wherein X of any of X₁-X₂ and X₅-X₈ can be A, C, T or G, in any combination;
d3) the nucleotide sequence of SEQ ID NO:63, which comprises the sequence X₁X₂X₃GTX₆X₇X₈GGTAAGTG (SEQ ID NO:344), wherein X of any of X₁-X₃ and X₆-X₈ can be A, C, T or G, in any combination;
e3) the nucleotide sequence of SEQ ID NO:64, which comprises the sequence X₁X₂X₃X₄TTX₇X₈GGTAAGTG (SEQ ID NO:345), wherein X of any of X₁-X₄ and X₇-X₅ can be A, C, T or G, in any combination;
f3) the nucleotide sequence of SEQ ID NO:65, which comprises the sequence X₁X₂X₃X₄TTX₇X₈GGTAAGTG (SEQ ID NO:345), wherein X of any of X₁-X₄ and X₇-X₈ can be A, C, T or G, in any combination;
g3) the nucleotide sequence of SEQ ID NO:66, which comprises the sequence X₁X₂X₃X₄X₆TAX₈GGTAATGTG (SEQ ID NO:346), wherein X of any of X₁-X₅ and X₈ can be A, C, T or G, in any combination;
h3) the nucleotide sequence of SEQ ID NO:67, which comprises the sequence X₁X₂X₃X₄X₆TAX₈GGTAATGTG (SEQ ID NO:346), wherein X of any of X₁-X₅ and X₈ can be A, C, T or G, in any combination;
i3) the nucleotide sequence of SEQ ID NO:68, which comprises the sequence AGCGAATAGGTAATAC (SEQ ID NO:347);
j3) the nucleotide sequence of SEQ ID NO:69, which comprises the sequence AGCGAATAGGTAATAC (SEQ ID NO:347);
k3) the nucleotide sequence of SEQ ID NO:70, which comprises the sequence CGAGGGCAGGTAATAA (SEQ ID NO:348);
l3) the nucleotide sequence of SEQ ID NO:71, which comprises the sequence CGAGGGCAGGTAATAA (SEQ ID NO:348);
m3) the nucleotide sequence of SEQ ID NO:72, which comprises the sequence GGTGCCGAGGTAATAT (SEQ ID NO:349);
n3) the nucleotide sequence of SEQ ID NO:73, which comprises the sequence GGTGCCGAGGTAATAT (SEQ ID NO:349);
o3) the nucleotide sequence of SEQ ID NO:74, which comprises the sequence GGTGACTAGGTAATAC (SEQ ID NO:350);
p3) the nucleotide sequence of SEQ ID NO:75, which comprises the sequence GGTGACTAGGTAATAC (SEQ ID NO:350);
q3) the nucleotide sequence of SEQ ID NO:76, which comprises the sequence CGAGGGCAGGTAATAT (SEQ ID NO:351);
r3) the nucleotide sequence of SEQ ID NO:77, which comprises the sequence CGAGGGCAGGTAATAT (SEQ ID NO:351);
s3) the nucleotide sequence of SEQ ID NO:78, which comprises the sequence AGCGCAGAGGTAATAA (SEQ ID NO:352);
t3) the nucleotide sequence of SEQ ID NO:79, which comprises the sequence AGCGCAGAGGTAATAA (SEQ ID NO:352);
u3) the nucleotide sequence of SEQ ID NO:80, which comprises the sequence AGCGAATAGGTAAGTC (SEQ ID NO:353);
v3) the nucleotide sequence of SEQ ID NO:81, which comprises the sequence AGCGAATAGGTAAGTC (SEQ ID NO:353);
w3) the nucleotide sequence of SEQ ID NO:82, which comprises the sequence CGAGGGCAGGTAAGTA (SEQ ID NO:354);
x3) the nucleotide sequence of SEQ ID NO:83, which comprises the sequence CGAGGGCAGGTAAGTA (SEQ ID NO:354);
y3) the nucleotide sequence of SEQ ID NO:84, which comprises the sequence GGTGCCGAGGTAAGTT (SEQ ID NO:355);
z3) the nucleotide sequence of SEQ ID NO:85, which comprises the sequence GGTGCCGAGGTAAGTT (SEQ ID NO:355);
a4) the nucleotide sequence of SEQ ID NO:86, which comprises the sequence GGTGACTAGGTAAGTC (SEQ ID NO:356);
b4) the nucleotide sequence of SEQ ID NO:87, which comprises the sequence GGTGACTAGGTAAGTC (SEQ ID NO:356);
c4) the nucleotide sequence of SEQ ID NO:88, which comprises the sequence CGAGGGCAGGTAAGTT (SEQ ID NO:357);
d4) the nucleotide sequence of SEQ ID NO:89, which comprises the sequence CGAGGGCAGGTAAGTT (SEQ ID NO:357);
e4) the nucleotide sequence of SEQ ID NO:90, which comprises the sequence AGCGCAGAGGTAAGTA (SEQ ID NO:358);
f4) the nucleotide sequence of SEQ ID NO:91, which comprises the sequence AGCGCAGAGGTAAGTA (SEQ ID NO:358);
g4) the nucleotide sequence of SEQ ID NO:10 (657G);
h4) the nucleotide sequence of SEQ ID NO:11 (657G);
i4) the nucleotide sequence of SEQ ID NO:12 (658T);
j4) the nucleotide sequence of SEQ ID NO:13 (658T);
k4) the nucleotide sequence of SEQ ID NO:18 (S1+657G);
14) the nucleotide sequence of SEQ ID NO:20 (S1+657G);
m4) the nucleotide sequence of SEQ ID NO:19 (S1+658T);
n4) the nucleotide sequence of SEQ ID NO:21 (S1+658T); and
o4) any combination of a1 through n4 above.

6. The cell of claim 1, wherein said first heterologous nucleic acid construct and/or said second heterologous nucleic acid construct comprises two or more second intronic nucleotide sequences.

7. The cell of claim 1, wherein said first heterologous nucleic acid construct and/or said second heterologous nucleic acid construct comprises two or more second intronic nucleotide sequences selected from the group consisting of:
a) second intronic nucleotide sequences in tandem within said first nucleotide sequence,
b) second intronic nucleotide sequences spaced at least 25 base pairs apart within said first nucleotide sequence,
c) second intronic nucleotide sequences spaced at least 50 base pairs apart within said first nucleotide sequence,
d) second intronic nucleotide sequences spaced at least 75 base pairs apart within said first nucleotide sequence,
e) second intronic nucleotide sequences spaced at least 100 base pairs apart within said first nucleotide sequence,
f) second intronic nucleotide sequences spaced at least 200 base pairs apart within said first nucleotide sequence,
g) second intronic nucleotide sequences spaced at least 300 base pairs apart within said first nucleotide sequence,
h) second intronic nucleotide sequences wherein a primary second intronic nucleotide sequence is located between a promoter and said first nucleotide sequence and a secondary second intronic nucleotide sequence is located within an open reading frame of said first nucleotide sequence; and
i) second intronic nucleotide sequences wherein a primary second intronic nucleotide sequence is located between said open reading frame and a poly A signal in said first nucleotide sequence and a secondary second intronic nucleotide sequence is located within said open reading frame of said first nucleotide sequence.

8. The cell of claim 6, wherein said two or more second intronic nucleotide sequences are the same within said first or second nucleic acid construct.

9. The cell of claim 6, wherein said two or more second intronic nucleotide sequences are each different from one another within said first or second nucleic acid construct.

10. The cell of claim 1, wherein said first heterologous nucleic acid construct and/or said second heterologous nucleic acid construct comprises two or more first nucleotide sequences.

11. The cell of claim 10, wherein said two or more first nucleotide sequences are the same within said first or second nucleic acid construct.

12. The cell of claim 10, wherein said two or more first nucleotide sequences are each different from one another with said first or second nucleic acid construct.

13. The cell of claim 1, wherein said first heterologous nucleic acid construct and/or said second heterologous nucleic acid construct further comprises a promoter that directs expression of said first nucleotide sequence(s) of said first heterologous nucleic acid construct and/or said second heterologous nucleic acid construct.

14. The cell of claim 13, wherein said second intronic nucleotide sequence of said first heterologous nucleic acid construct and/or said second heterologous nucleic acid construct is positioned between said promoter and said first nucleotide sequence(s).

15. The cell of claim 1, wherein said second intronic nucleotide sequence of said first heterologous nucleic acid construct and/or said second heterologous nucleic acid construct is positioned within an open reading frame of said first nucleotide sequence(s).

16. The cell of claim 1, wherein said second intronic nucleotide sequence of said first heterologous nucleic acid construct and/or said second heterologous nucleic acid construct is positioned within the 5′ one-third of an open reading frame of said first nucleotide sequence(s).

17. The cell of claim 1, wherein said second intronic nucleotide sequence of said first heterologous nucleic acid construct and/or said second heterologous nucleic acid construct is positioned within the middle one-third of said open reading frame of said first nucleotide sequence(s).

18. The cell of claim 1, wherein said second intronic nucleotide sequence of said first heterologous nucleic acid construct and/or said second heterologous nucleic acid construct is positioned within the 3′ one-third of said open reading frame of said first nucleotide sequence(s).

19. The cell of claim 1, wherein said second intronic nucleotide sequence of said first heterologous nucleic acid construct and/or said second heterologous nucleic acid construct is positioned between said open reading frame and a poly A signal in said first nucleotide sequence(s).

20. An isolated nucleic acid comprising:
A) a first nucleotide sequence encoding a nucleotide sequence of interest (NOI); and
B) a second intronic nucleotide sequence operably associated with said first nucleotide sequence and wherein said second intronic nucleotide sequence is heterologous to said first nucleotide sequence and further wherein said second intronic nucleotide sequence is selected from the group consisting of:
a1) the nucleotide sequence of SEQ ID NO:92 (S0 257 by intron);
b1) the nucleotide sequence of SEQ ID NO:2 (S0−GT);
c1) the nucleotide sequence of SEQ ID NO:1 (S0−CT);
d1) the nucleotide sequence of SEQ ID NO:4 (S0−GT+CT);
e1) the nucleotide sequence of SEQ ID NO:3 (S1);
f1) the nucleotide sequence of SEQ ID NO:5 (S1+CT);
g1) the nucleotide sequence of SEQ ID NO:6 (M3);
h1) the nucleotide sequence of SEQ ID NO:7 (M3+CT);
i1) the nucleotide sequence of SEQ ID NO:8 (M6);
j1) the nucleotide sequence of SEQ ID NO:9 (M6+CT);
k1) the nucleotide sequence of SEQ ID NO:14 (M3+S1);
l1) the nucleotide sequence of SEQ ID NO:16 (M3+S1+CT);
m1) the nucleotide sequence of SEQ ID NO:15 (M6+S1);
n1) the nucleotide sequence of SEQ ID NO:17 (M6+S1+CT);
o1) the nucleotide sequence of SEQ ID NO:22, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;
p1) the nucleotide sequence of SEQ ID NO:23, which comprises the sequence X₁X₂X₃X₄X₅X₆X₇X₈X₉GTX₁₀X₁₁X₁₂X₁₃X₁₄ (SEQ ID NO:324), wherein X of any of X₁-X₉ and X₁₀-X₁₄ can be A, C, T or G, in any combination;
q1) the nucleotide sequence of SEQ ID NO:24, which comprises the sequence X₁GX₃GX₅X₆X₇AGGTAATAX₁₄ (SEQ ID NO:325), wherein X of any of X₁, X₃-X₇ and X₁₄ can be A, C, T or G, in any combination;
r1) the nucleotide sequence of SEQ ID NO:25, which comprises the sequence X₁GX₃GX₆X₆X₇AGGTAATAX₁₄ (SEQ ID NO:325), wherein X of any of X₁, X₃-X₇ and X₁₄ can be A, C, T or G, in any combination;
s1) the nucleotide sequence of SEQ ID NO:26, which comprises the sequence X₁GX₃GX₆X₆X₇AGGTAAGTX₁₄ (SEQ ID NO:326), wherein X of any of X₁, X₃, X₅-X₇ and X₁₄ can be A, C, T or G, in any combination;
t1) the nucleotide sequence of SEQ ID NO:27, which comprises the sequence X₁GX₃GX₆X₆X₇AGGTAAGTX₁₄ (SEQ ID NO:326), wherein X of any of X₁, X₃, X₅-X₇ and X₁₄ can be A, C, T or G, in any combination;
u1) the nucleotide sequence of SEQ ID NO:28, which comprises the sequence X₁X₂X₃X₄X₆X₆AAGGTAATAG (SEQ ID NO:327), wherein X of any of X₁,-X₆ can be A, C, T or G, in any combination;
v1) the nucleotide sequence of SEQ ID NO:29, which comprises the sequence X₁X₂X₃X₄X₆X₆AAGGTAATAG (SEQ ID NO:327), wherein X of any of X₁,-X₆ can be A, C, T or G, in any combination;
w1) the nucleotide sequence of SEQ ID NO:30, which comprises the sequence X₁GX₃X₄X₆X₆X₇AGGTAATAG (SEQ ID NO:328), wherein X of any of X₁ and X₃-X₇ can be A, C, T or G, in any combination;
x1) the nucleotide sequence of SEQ ID NO:31, which comprises the sequence X₁GX₃X₄X₆X₆X₇AGGTAATAG (SEQ ID NO:328), wherein X of any of X₁, and X₃-X₇ can be A, C, T or G, in any combination;
y1) the nucleotide sequence of SEQ ID NO:32, which comprises the sequence X₁X₂GX₄X₆X₆X₇AGGTAATAG (SEQ ID NO:329), wherein X of any of X₁, X₂ and X₄-X₇ can be A, C, T or G, in any combination;
z1) the nucleotide sequence of SEQ ID NO:33, which comprises the sequence X₁X₂GX₄X₆X₆X₇AGGTAATAG (SEQ ID NO:329), wherein X of any of X₁, X₂ and X₄-X₇ can be A, C, T or G, in any combination;
a2) the nucleotide sequence of SEQ ID NO:34, which comprises the sequence X₁X₂X₃GX₆X₆X₇AGGTAATAG (SEQ ID NO:330), wherein X of any of X₁-X₃ and X₅-X₇ can be A, C, T or G, in any combination;
b2) the nucleotide sequence of SEQ ID NO:35, which comprises the sequence X₁X₂X₃GX₆X₆X₇AGGTAATAG (SEQ ID NO:330), wherein X of any of X₁-X₃ and X₅-X₇ can be A, C, T or G, in any combination;
c2) the nucleotide sequence of SEQ ID NO:36, which comprises the sequence X₁X₂X₃X₄TX₆X₇AGGTAATAG (SEQ ID NO:331), wherein X of any of X₁-X₄ and X₆-X₇ can be A, C, T or G, in any combination;
d2) the nucleotide sequence of SEQ ID NO:37, which comprises the sequence X₁X₂X₃X₄TX₈X₇AGGTAATAG (SEQ ID NO:331), wherein X of any of X₁-X₄ and X₆-X₇ can be A, C, T or G, in any combination;
e2) the nucleotide sequence of SEQ ID NO:38, which comprises the sequence X₁GGX₄X₅X₈X₇X₈GGTAATAG (SEQ ID NO:332), wherein X of any of X₁ and X₄-X₈ can be A, C, T or G, in any combination;
f2) the nucleotide sequence of SEQ ID NO:39, which comprises the sequence X₁GGX₄X₅X₈X₇X₈GGTAATAG (SEQ ID NO:332), wherein X of any of X₁ and X₄-X₅ can be A, C, T or G, in any combination;
g2) the nucleotide sequence of SEQ ID NO:40, which comprises the sequence X₁X₂GGX₅X₈X₇X₈GGTAATAG (SEQ ID NO:333), wherein X of any of X₁, X₂ and X₅-X₈ can be A, C, T or G, in any combination;
h2) the nucleotide sequence of SEQ ID NO:41, which comprises the sequence X₁X₂GGX₅X₈X₇X₈GGTAATAG (SEQ ID NO:333), wherein X of any of X₁, X₂ and X₅-X₈ can be A, C, T or G, in any combination;
i2) the nucleotide sequence of SEQ ID NO:42, which comprises the sequence X₁X₂X₃GTX₈X₇X₈GGTAATAG (SEQ ID NO:334), wherein X of any of X₁-X₃ and X₆-X₈ can be A, C, T or G, in any combination;
j2) the nucleotide sequence of SEQ ID NO:43, which comprises the sequence X₁X₂X₃GTX₈X₇X₈GGTAATAG (SEQ ID NO:334), wherein X of any of X₁-X₃ and X₆-X₈ can be A, C, T or G, in any combination;
k2) the nucleotide sequence of SEQ ID NO:44, which comprises the sequence X₁X₂X₃X₄TTX₇X₈GGTAATAG (SEQ ID NO:335), wherein X of any of X₁-X₄ and X₇-X₈ can be A, C, T or G, in any combination;
l2) the nucleotide sequence of SEQ ID NO:45, which comprises the sequence X₁X₂X₃X₄TTX₇X₈GGTAATAG (SEQ ID NO:335), wherein X of any of X₁-X₄ and X₇-X₈ can be A, C, T or G, in any combination;
m2) the nucleotide sequence of SEQ ID NO:46, which comprises the sequence X₁X₂X₃X₄X₅TAX₈GGTAATAG (SEQ ID NO:336), wherein X of any of X₁-X₅ and X₈ can be A, C, T or G, in any combination;
n2) the nucleotide sequence of SEQ ID NO:47, which comprises the sequence X₁X₂X₃X₄X₅TAX₈GGTAATAG (SEQ ID NO:336), wherein X of any of X₁-X₅ and Xₛ can be A, C, T or G, in any combination;
o2) the nucleotide sequence of SEQ ID NO:48, which comprises the sequence X₁X₂X₃X₄X₅X₈AAGGTAAGTG (SEQ ID NO:337), wherein X of any of X₁-X₆ can be A, C, T or G, in any combination;
p2) the nucleotide sequence of SEQ ID NO:49, which comprises the sequence X₁X₂X₃X₄X₅X₆AAGGTAAGTG (SEQ ID NO:337), wherein X of any of X₁-X₆ can be A, C, T or G, in any combination;
q2) the nucleotide sequence of SEQ ID NO:50, which comprises the sequence X₁GX₃X₄X₅X₈X₇AGGTAAGTG (SEQ ID NO:338), wherein X of any of X₁ and X₃-X₇ can be A, C, T or G, in any combination;
r2) the nucleotide sequence of SEQ ID NO:51, which comprises the sequence X₁GX₃X₄X₅X₆X₇AGGTAAGTG (SEQ ID NO:338), wherein X of any of X₁ and X₃-X₇ can be A, C, T or G, in any combination;
s2) the nucleotide sequence of SEQ ID NO:52, in any combination, which comprises the sequence X₁X₂GX₄X₅X₈X₇AGGTAAGTG (SEQ ID NO:339), wherein X of any of X₁-X₂ and X₄-X₇ can be A, C, T or G;
t2) the nucleotide sequence of SEQ ID NO:53, which comprises the sequence X₁X₂GX₄X₅X₆X₇AGGTAAGTG (SEQ ID NO:339), wherein X of any of X₁-X₂ and X₄-X₇ can be A, C, T or G, in any combination;
u2) the nucleotide sequence of SEQ ID NO:54, which comprises the sequence X₁X₂X₃GX₅X₈X₇AGGTAAGTG (SEQ ID NO:340), wherein X of any of X₁-X₃ and X₅-X₇ can be A, C, T or G, in any combination;
v2) the nucleotide sequence of SEQ ID NO:55, which comprises the sequence X₁X₂X₃GX₅X₈X₇AGGTAAGTG (SEQ ID NO:340), wherein X of any of X₁-X₃ and X₅-X₇ can be A, C, T or G, in any combination;
w2) the nucleotide sequence of SEQ ID NO:56, which comprises the sequence X₁X₂X₃X₄TX₆X₇AGGTAAGTG (SEQ ID NO:341), wherein X of any of X₁-X₄ and X₆-X₇ can be A, C, T or G, in any combination;
x2) the nucleotide sequence of SEQ ID NO:57, which comprises the sequence X₁X₂X₃X₄TX₈X₇AGGTAAGTG (SEQ ID NO:341), wherein X of any of X₁-X₄ and X₆-X₇ can be A, C, T or G, in any combination;
y2) the nucleotide sequence of SEQ ID NO:58, which comprises the sequence X₁GGX₄X₅X₈X₇X₈GGTAAGTG (SEQ ID NO:342), wherein X of any of X₁ and X₄-X₈ can be A, C, T or G, in any combination;
z2) the nucleotide sequence of SEQ ID NO:59, which comprises the sequence X₁GGX₄X₅X₆X₇X₈GGTAAGTG (SEQ ID NO:342), wherein X of any of X₁ and X₄-X₈ can be A, C, T or G, in any combination;
a3) the nucleotide sequence of SEQ ID NO:60, which comprises the sequence X₁X₂GGX₅X₈X₇X₈GGTAAGTG (SEQ ID NO:343), wherein X of any of X₁-X₂ and X₅-X₈ can be A, C, T or G, in any combination;
b3) the nucleotide sequence of SEQ ID NO:61, which comprises the sequence X₁X₂GGX₅X₈X₇X₈GGTAAGTG (SEQ ID NO:343), wherein X of any of X₁-X₂ and X₅-X₈ can be A, C, T or G, in any combination;
c3) the nucleotide sequence of SEQ ID NO:62, which comprises the sequence X₁X₂X₃GTX₈X₇X₈GGTAAGTG (SEQ ID NO:344), wherein X of any of X₁-X₂ and X₅-X₈ can be A, C, T or G, in any combination;
d3) the nucleotide sequence of SEQ ID NO:63, which comprises the sequence X₁X₂X₃GTX₈X₇X₈GGTAAGTG (SEQ ID NO:344), wherein X of any of X₁-X₃ and X₆-X₈ can be A, C, T or G, in any combination;
e3) the nucleotide sequence of SEQ ID NO:64, which comprises the sequence X₁X₂X₃X₄TTX₇X₈GGTAAGTG (SEQ ID NO:345), wherein X of any of X₁-X₄ and X₇-X₈ can be A, C, T or G, in any combination;
f3) the nucleotide sequence of SEQ ID NO:65, which comprises the sequence X₁X₂X₃X_{I}TTX₇X₈GGTAAGTG (SEQ ID NO:345), wherein X of any of X₁-X₁and X₇-X₈ can be A, C, T or G, in any combination;
g3) the nucleotide sequence of SEQ ID NO:66, which comprises the sequence X₁X₂X₃X₄X₅TAX₈GGTAATGTG (SEQ ID NO:346), wherein X of any of X₁-X₅ and X₅ can be A, C, T or G, in any combination;
h3) the nucleotide sequence of SEQ ID NO:67, which comprises the sequence X₁X₂X₃X₄X₅TAX₈GGTAATGTG (SEQ ID NO:346), wherein X of any of X₁-X₅ and X₈ can be A, C, T or G, in any combination;
i3) the nucleotide sequence of SEQ ID NO:68, which comprises the sequence AGCGAATAGGTAATAC (SEQ ID NO:347);
j3) the nucleotide sequence of SEQ ID NO:69, which comprises the sequence AGCGAATAGGTAATAC (SEQ ID NO:347);
k3) the nucleotide sequence of SEQ ID NO:70, which comprises the sequence CGAGGGCAGGTAATAA (SEQ ID NO:348);
l3) the nucleotide sequence of SEQ ID NO:71, which comprises the sequence CGAGGGCAGGTAATAA (SEQ ID NO:348);
m3) the nucleotide sequence of SEQ ID NO:72, which comprises the sequence GGTGCCGAGGTAATAT (SEQ ID NO:349);
n3) the nucleotide sequence of SEQ ID NO:73, which comprises the sequence GGTGCCGAGGTAATAT (SEQ ID NO:349);
o3) the nucleotide sequence of SEQ ID NO:74, which comprises the sequence GGTGACTAGGTAATAC (SEQ ID NO:350);
p3) the nucleotide sequence of SEQ ID NO:75, which comprises the sequence GGTGACTAGGTAATAC (SEQ ID NO:350);
q3) the nucleotide sequence of SEQ ID NO:76, which comprises the sequence CGAGGGCAGGTAATAT (SEQ ID NO:351);
r3) the nucleotide sequence of SEQ ID NO:77, which comprises the sequence CGAGGGCAGGTAATAT (SEQ ID NO:351);
s3) the nucleotide sequence of SEQ ID NO:78, which comprises the sequence AGCGCAGAGGTAATAA (SEQ ID NO:352);
t3) the nucleotide sequence of SEQ ID NO:79, which comprises the sequence AGCGCAGAGGTAATAA (SEQ ID NO:352);
u3) the nucleotide sequence of SEQ ID NO:80, which comprises the sequence AGCGAATAGGTAAGTC (SEQ ID NO:353);
v3) the nucleotide sequence of SEQ ID NO:81, which comprises the sequence AGCGAATAGGTAAGTC (SEQ ID NO:353);
w3) the nucleotide sequence of SEQ ID NO:82, which comprises the sequence CGAGGGCAGGTAAGTA (SEQ ID NO:354);
x3) the nucleotide sequence of SEQ ID NO:83, which comprises the sequence CGAGGGCAGGTAAGTA (SEQ ID NO:354);
y3) the nucleotide sequence of SEQ ID NO:84, which comprises the sequence GGTGCCGAGGTAAGTT (SEQ ID NO:355);
z3) the nucleotide sequence of SEQ ID NO:85, which comprises the sequence GGTGCCGAGGTAAGTT (SEQ ID NO:355);
a4) the nucleotide sequence of SEQ ID NO:86, which comprises the sequence GGTGACTAGGTAAGTC (SEQ ID NO:356);
b4) the nucleotide sequence of SEQ ID NO:87. which comprises the sequence GGTGACTAGGTAAGTC (SEQ ID NO:356);
c4) the nucleotide sequence of SEQ ID NO:88, which comprises the sequence CGAGGGCAGGTAAGTT (SEQ ID NO:357);
d4) the nucleotide sequence of SEQ ID NO:89, which comprises the sequence CGAGGGCAGGTAAGTT (SEQ ID NO:357);
e4) the nucleotide sequence of SEQ ID NO:90, which comprises the sequence AGCGCAGAGGTAAGTA (SEQ ID NO:358);
f4) the nucleotide sequence of SEQ ID NO:91, which comprises the sequence AGCGCAGAGGTAAGTA (SEQ ID NO:358);
g4) the nucleotide sequence of SEQ ID NO:10 (657G);
h4) the nucleotide sequence of SEQ ID NO:11 (657G);
i4) the nucleotide sequence of SEQ ID NO:12 (658T);
j4) the nucleotide sequence of SEQ ID NO:13 (658T);
k4) the nucleotide sequence of SEQ ID NO:18 (51+657G);
l4) the nucleotide sequence of SEQ ID NO:20 (S1+657G);
m4) the nucleotide sequence of SEQ ID NO:19 (S1+658T);
n4) the nucleotide sequence of SEQ ID NO:21 (S1+658T); and
o4) any combination of a1 through n4 above.

21. The isolated nucleic acid of claim 20, comprising two or more second intronic nucleotide sequences.

22. The isolated nucleic acid of claim 20, comprising two or more second intronic nucleotide sequences selected from the group consisting of:
a) second intronic nucleotide sequences in tandem within said first nucleotide sequence,
b) second intronic nucleotide sequences spaced at least 25 base pairs apart within said first nucleotide sequence,
c) second intronic nucleotide sequences spaced at least 50 base pairs apart within said first nucleotide sequence,
d) second intronic nucleotide sequences spaced at least 75 base pairs apart within said first nucleotide sequence,
e) second intronic nucleotide sequences spaced at least 100 base pairs apart within said first nucleotide sequence,
f) second intronic nucleotide sequences spaced at least 200 base pairs apart within said first nucleotide sequence,
g) second intronic nucleotide sequences spaced at least 300 base pairs apart within said first nucleotide sequence,
h) second intronic nucleotide sequences wherein a primary second intronic nucleotide sequence is located between a promoter and an open reading frame of said first nucleotide sequence and a secondary second intronic nucleotide sequence is located within an open reading frame of said first nucleotide sequence; and
i) second intronic nucleotide sequences wherein a primary second intronic nucleotide sequence is located between an open reading frame and a poly A signal in said first nucleotide sequence and a secondary second intronic nucleotide sequence is located within said open reading frame of said first nucleotide sequence.

23. The nucleic acid of claim 20, wherein said first nucleotide sequence encodes a nucleotide sequence of interest (NOI) selected from the group consisting of:
a) a nucleotide sequence encoding a protein or peptide;
b) a nucleotide sequence encoding a product having activity as an interfering RNA;
c) a nucleotide sequence encoding a product having enzymatic activity as an RNA;
d) a nucleotide sequence encoding a ribozyme;
e) a nucleotide sequence encoding an antisense sequence;
f) a nucleotide sequence encoding a small nuclear RNA (snRNA); and
g) any combination of (a)-(f) above

24. The nucleic acid of claim 20, comprising two or more first nucleotide sequences.

25. The nucleic acid of claim 24, wherein said two or more first nucleotide sequences are the same.

26. The nucleic acid of claim 24, wherein said two or more first nucleotide sequences are each different from one another.

27. The nucleic acid of claim 20, further comprising a promoter that directs expression of said first nucleotide sequence.

28. The nucleic acid of claim 27, wherein said second intronic nucleotide sequence is positioned between the promoter and an open reading frame of said first nucleotide sequence.

29. The nucleic acid of claim 20, wherein said second intronic nucleotide sequence is positioned within said open reading frame of said first nucleotide sequence.

30. The nucleic acid of claim 29, wherein said second intronic nucleotide sequence is positioned within the 5′ one-third of said open reading frame of said first nucleotide sequence.

31. The nucleic acid of claim 29, wherein said second intronic nucleotide sequence is positioned within the middle one-third of said open reading frame of said first nucleotide sequence.

32. The nucleic acid of claim 29, wherein said second intronic nucleotide sequence is positioned within the 3′ one-third of said open reading frame of said first nucleotide sequence.

33. The nucleic acid of claim 20, wherein said second intronic nucleotide sequence is positioned between said open reading frame and a poly A signal in said first nucleotide sequence.

34. A vector comprising the nucleic acid of claim 20.

35. A cell comprising the nucleic acid of claim 20.

36. A cell comprising the vector of claim 34.

37. A composition comprising the nucleic acid of claim 20 in a pharmaceutically acceptable carrier.

38. A composition comprising the cell of claim 1 in a pharmaceutically acceptable carrier.

39. A composition comprising the vector of claim 34 in a pharmaceutically acceptable carrier.

40. A method of producing a functional RNA encoded by said first nucleotide sequence of said first heterologous nucleic acid construct or a functional RNA encoded by said.first nucleotide sequence of said second heterologous nucleic acid construct in the cell of claim 1, comprising:
introducing into said cell a blocking oligonucleotide and/or small molecule that blocks a member of said second set of splice elements of said second intronic nucleotide sequence of said first heterologous nucleic acid construct or of said second heterologous nucleic acid construct, thereby producing the functional RNA encoded by said first nucleotide sequence of said first heterologous nucleic acid construct or encoded by first nucleotide sequence of said second heterologous nucleic acid construct in said cell.

41. A method of producing a first functional RNA encoded by said first nucleotide sequence of said first heterologous nucleic acid construct and producing a second functional RNA encoded by said first nucleotide sequence of said second heterologous nucleic acid construct in the cell of claim 1, wherein said first functional RNA and said second functional RNA are different from each other, comprising:
a) introducing into said cell a first blocking oligonucleotide and/or first small molecule that blocks a member of said second set of splice elements of said second intronic nucleotide sequence of said first heterologous nucleic acid construct, thereby producing said first functional RNA encoded by said first nucleotide sequence of said first heterologous nucleic acid construct in said cell, and
b) introducing into said cell a second blocking oligonucleotide and/or second small molecule that blocks a member of said second set of splice elements of said second intronic nucleotide sequence of said second heterologous nucleic acid construct, thereby producing said second functional RNA encoded by said first nucleotide sequence of said second heterologous nucleic acid construct in said cell,
wherein said second intronic nucleotide sequence of said first heterologous nucleic acid construct and said second intronic nucleotide sequence of said second heterologous nucleic acid construct are different from each other and wherein said first blocking oligonucleotide and/or first small molecule and said second blocking oligonucleotide and/or second small molecule are different from each other.

42. A method for producing a functional RNA encoded by said first nucleotide sequence of said isolated nucleic acid of claim 20, comprising contacting a blocking oligonucleotide and/or small molecule with the isolated nucleic acid under conditions that permit splicing, wherein the blocking oligonucleotide and/or small molecule blocks a member of said second set of splice elements of said second intronic nucleotide sequence, thereby producing said functional RNA encoded by said first nucleotide sequence.

43. The method of claim 42, wherein the blocking oligonucleotide and/or small molecule is introduced into a cell comprising the isolated nucleic acid.

44. The method of claim 40, wherein the blocking oligonucleotide does not activate RNase H.

45. The method of claim 40, wherein the blocking oligonucleotide comprises a modified internucleotide bridging phosphate residue selected from the group consisting of methyl phosphorothioates, phosphoromorpholidates, phosphoropiperazidates, phosphoramidates and any combination thereof.

46. The method of claim 40, wherein the blocking oligonucleotide comprises a nucleotide having a loweralkyl substituent at the 2′ position thereof.

47. The method of claim 40, wherein the blocking oligonucleotide is from about eight to about 50 nucleotides in length.

48. The method of claim 40, wherein the cell is in a subject.

49. The method of claim 48, wherein the subject is a human.


SEQUENCE LISTING (385 sequences)

SEQ ID NO: 1 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtaatagcaatatttctgcatataaatatttagtccaagctaggcccttttgctaatcatgttcatacctcttatcttcctcccacag

SEQ ID NO: 2 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttagtccaagctaggcccttttgctaatcatgttcatacctcttatcttcctcccacag

SEQ ID NO: 3 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtaagtgcaatatttctgcatataaatatttagtccaagctaggcccttttgctaatcatgttcatacctcttatcttcctcccacag

SEQ ID NO: 4 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtaagtgcaatatttctgcatataaatatttagtccaagctaggcccttttgctaatcatgttcatacctcttatcttcctcccacag

SEQ ID NO: 5 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgccaataggtaagtgcaatatttctgcatataaatatttagtccaagctaggcccttttgctaatcatgttcatacctcttatcttcctcccacag

SEQ ID NO: 6 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtgagtgcaatatttctgcatataaatatttagtccaagctaggcccttttgctaatcatgttcatacctcttatcttcctcccacag

SEQ ID NO: 7 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtgagtgcaatatttctgcatataaatatttagtccaagctaggcccttttgctaatcatgttcatacctcttatcttcctcccacag

SEQ ID NO: 8 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaagggcaatatttctgcatataaatatttagtccaagctaggcccttttgctaatcatgttcatacctcttatcttcctcccacag

SEQ ID NO: 9 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtaagggcaatatttctgcatataaatatttagtccaagctaggcccttttgctaatcatgttcatacctcttatcttcctcccacag

SEQ ID NO: 10 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaagagcaatatttctgcatataaatatttagtccaagctaggcccttttgctaatcatgttcatacctcttatcttcctcccacag

SEQ ID NO: 11 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtaagagcaatatttctgcatataaatatttagtccaagctaggcccttttgctaatcatgttcatacctcttatcttcctcccacag

SEQ ID NO: 12 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaattgcaatatttctgcatataaatatttagtccaagctaggcccttttgctaatcatgttcatacctcttatcttcctcccacag

SEQ ID NO: 13 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtaattgcaatatttctgcatataaatatttagtccaagctaggcccttttgctaatcatgttcatacctcttatcttcctcccacag

SEQ ID NO: 14 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtgagtgcaatatttctgcatataaatatttagtccaagctaggcccttttgctaatcatgttcatacctcttatcttcctcccacag

SEQ ID NO: 15 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtaagggcaatatttctgcatataaatatttagtccaagctaggcccttttgctaatcatgttcatacctcttatcttcctcccacag

SEQ ID NO: 16 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgccaataggtgagtgcaatatttctgcatataaatatttagtccaagctaggcccttttgctaatcatgttcatacctcttatcttcctcccacag

SEQ ID NO: 17 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgccaataggtaagggcaatatttctgcatataaatatttagtccaagctaggcccttttgctaatcatgttcatacctcttatcttcctcccacag

SEQ ID NO: 18 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtaagagcaatatttctgcatataaatatttagtccaagctaggcccttttgctaatcatgttcatacctcttatcttcctcccacag

SEQ ID NO: 19 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtaattgcaatatttctgcatataaatatttagtccaagctaggcccttttgctaatcatgttcatacctcttatcttcctcccacag

SEQ ID NO: 20 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgccaataggtaagagcaatatttctgcatataaatatttagtccaagctaggcccttttgctaatcatgttcatacctcttatcttcctcccacag

SEQ ID NO: 21 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgccaataggtaattgcaatatttctgcatataaatatttagtccaagctaggcccttttgctaatcatgttcatacctcttatcttcctcccacag

SEQ ID NO: 22 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnnnnnngtnnnnncaatatttctgcatataaatatttagtccaagctaggcccttttgctaatcatgttcatacctcttatcttcctcccacag

SEQ ID NO: 23 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnnnnnngtnnnnncaatatttctgcatataaatatttagtccaagctaggcccttttgctaatcatgttcatacctcttatcttcctcccacag

SEQ ID NO: 24 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcngngnnnaggtaatancaatatttctgcatataaatatttagtccaagctaggcccttttgctaatcatgttcatacctcttatcttcctcccacag

SEQ ID NO: 25 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcngngnnnaggtaatancaatatttctgcatataaatatttagtccaagctaggcccttttgctaatcatgttcatacctcttatcttcctcccacag

SEQ ID NO: 26 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcngngnnnaggtaagtncaatatttctgcatataaatatttagtccaagctaggcccttttgctaatcatgttcatacctcttatcttcctcccacag

SEQ ID NO: 27 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcngngnnnaggtaagtncaatatttctgcatataaatatttagtccaagctaggcccttttgctaatcatgttcatacctcttatcttcctcccacag

SEQ ID NO: 28 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnnnaaggtaatagcaatatttctgcatataaatatttagtccaagctaggcccttttgctaatcatgttcatacctcttatcttcctcccacag

SEQ ID NO: 29 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnnnaaggtaatagcaatatttctgcatataaatatttagtccaagctaggcccttttgctaatcatgttcatacctcttatcttcctcccacag

SEQ ID NO: 30 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcngnnnnnaggtaatagcaatatttctgcatataaatatttagtccaagctaggcccttttgctaatcatgttcatacctcttatcttcctcccacag

SEQ ID NO: 31 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcngnnnnnaggtaatagcaatatttctgcatataaatatttagtccaagctaggcccttttgctaatcatgttcatacctcttatcttcctcccacag

SEQ ID NO: 32 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnngnnnnaggtaatagcaatatttctgcatataaatatttagtccaagctaggcccttttgctaatcatgttcatacctcttatcttcctcccacag

SEQ ID NO: 33 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnngnnnnaggtaatagcaatatttctgcatataaatatttagtccaagctaggcccttttgctaatcatgttcatacctcttatcttcctcccacag

SEQ ID NO: 34 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnngnnnaggtaatagcaatatttctgcatataaatatttagtccaagctaggcccttttgctaatcatgttcatacctcttatcttcctcccacag

SEQ ID NO: 35 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnngnnnaggtaatagcaatatttctgcatataaatatttagtccaagctaggcccttttgctaatcatgttcatacctcttatcttcctcccacag

SEQ ID NO: 36 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnntnnaggtaatagcaatatttctgcatataaatatttagtccaagctaggcccttttgctaatcatgttcatacctcttatcttcctcccacag

SEQ ID NO: 37 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnntnnaggtaatagcaatatttctgcatataaatatttagtccaagctaggcccttttgctaatcatgttcatacctcttatcttcctcccacag

SEQ ID NO: 38 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnggnnnnnggtaatagcaatatttctgcatataaatatttagtccaagctaggcccttttgctaatcatgttcatacctcttatcttcctcccacag

SEQ ID NO: 39 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnggnnnnnggtaatagcaatatttctgcatataaatatttagtccaagctaggcccttttgctaatcatgttcatacctcttatcttcctcccacag

SEQ ID NO: 40 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnggnnnnggtaatagcaatatttctgcatataaatatttagtccaagctaggcccttttgctaatcatgttcatacctcttatcttcctcccacag

SEQ ID NO: 41 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnggnnnnggtaatagcaatatttctgcatataaatatttagtccaagctaggcccttttgctaatcatgttcatacctcttatcttcctcccacag

SEQ ID NO: 42 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnngtnnnggtaatagcaatatttctgcatataaatatttagtccaagctaggcccttttgctaatcatgttcatacctcttatcttcctcccacag

SEQ ID NO: 43 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnngtnnnggtaatagcaatatttctgcatataaatatttagtccaagctaggcccttttgctaatcatgttcatacctcttatcttcctcccacag

SEQ ID NO: 44 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnttnnggtaatagcaatatttctgcatataaatatttagtccaagctaggcccttttgctaatcatgttcatacctcttatcttcctcccacag

SEQ ID NO: 45 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnttnnggtaatagcaatatttctgcatataaatatttagtccaagctaggcccttttgctaatcatgttcatacctcttatcttcctcccacag

SEQ ID NO: 46 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnntanggtaatagcaatatttctgcatataaatatttagtccaagctaggcccttttgctaatcatgttcatacctcttatcttcctcccacag

SEQ ID NO: 47 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnntanggtaatagcaatatttctgcatataaatatttagtccaagctaggcccttttgctaatcatgttcatacctcttatcttcctcccacag

SEQ ID NO: 48 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnnnaaggtaagtgcaatatttctgcatataaatatttagtccaagctaggcccttttgctaatcatgttcatacctcttatcttcctcccacag

SEQ ID NO: 49 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnnnaaggtaagtgcaatatttctgcatataaatatttagtccaagctaggcccttttgctaatcatgttcatacctcttatcttcctcccacag

SEQ ID NO: 50 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcngnnnnnaggtaagtgcaatatttctgcatataaatatttagtccaagctaggcccttttgctaatcatgttcatacctcttatcttcctcccacag

SEQ ID NO: 51 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcngnnnnnaggtaagtgcaatatttctgcatataaatatttagtccaagctaggcccttttgctaatcatgttcatacctcttatcttcctcccacag

SEQ ID NO: 52 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnngnnnnaggtaagtgcaatatttctgcatataaatatttagtccaagctaggcccttttgctaatcatgttcatacctcttatcttcctcccacag

SEQ ID NO: 53 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnngnnnnaggtaagtgcaatatttctgcatataaatatttagtccaagctaggcccttttgctaatcatgttcatacctcttatcttcctcccacag

SEQ ID NO: 54 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnngnnnaggtaagtgcaatatttctgcatataaatatttagtccaagctaggcccttttgctaatcatgttcatacctcttatcttcctcccacag

SEQ ID NO: 55 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnngnnnaggtaagtgcaatatttctgcatataaatatttagtccaagctaggcccttttgctaatcatgttcatacctcttatcttcctcccacag

SEQ ID NO: 56 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnntnnaggtaagtgcaatatttctgcatataaatatttagtccaagctaggcccttttgctaatcatgttcatacctcttatcttcctcccacag

SEQ ID NO: 57 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnntnnaggtaagtgcaatatttctgcatataaatatttagtccaagctaggcccttttgctaatcatgttcatacctcttatcttcctcccacag

SEQ ID NO: 58 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnggnnnnnggtaagtgcaatatttctgcatataaatatttagtccaagctaggcccttttgctaatcatgttcatacctcttatcttcctcccacag

SEQ ID NO: 59 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnggnnnnnggtaagtgcaatatttctgcatataaatatttagtccaagctaggcccttttgctaatcatgttcatacctcttatcttcctcccacag

SEQ ID NO: 60 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnggnnnnggtaagtgcaatatttctgcatataaatatttagtccaagctaggcccttttgctaatcatgttcatacctcttatcttcctcccacag

SEQ ID NO: 61 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnggnnnnggtaagtgcaatatttctgcatataaatatttagtccaagctaggcccttttgctaatcatgttcatacctcttatcttcctcccacag

SEQ ID NO: 62 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnngtnnnggtaagtgcaatatttctgcatataaatatttagtccaagctaggcccttttgctaatcatgttcatacctcttatcttcctcccacag

SEQ ID NO: 63 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnngtnnnggtaagtgcaatatttctgcatataaatatttagtccaagctaggcccttttgctaatcatgttcatacctcttatcttcctcccacag

SEQ ID NO: 64 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnttnnggtaagtgcaatatttctgcatataaatatttagtccaagctaggcccttttgctaatcatgttcatacctcttatcttcctcccacag

SEQ ID NO: 65 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnttnnggtaagtgcaatatttctgcatataaatatttagtccaagctaggcccttttgctaatcatgttcatacctcttatcttcctcccacag

SEQ ID NO: 66 - DNA, 248 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcnnnnntanggtaatgtgcaatatttctgcatataaatatttagtccaagctaggcccttttgctaatcatgttcatacctcttatcttcctcccacag

SEQ ID NO: 67 - DNA, 248 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcnnnnntanggtaatgtgcaatatttctgcatataaatatttagtccaagctaggcccttttgctaatcatgttcatacctcttatcttcctcccacag

SEQ ID NO: 68 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcagcgaataggtaataccaatatttctgcatataaatatttagtccaagctaggcccttttgctaatcatgttcatacctcttatcttcctcccacag

SEQ ID NO: 69 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcagcgaataggtaataccaatatttctgcatataaatatttagtccaagctaggcccttttgctaatcatgttcatacctcttatcttcctcccacag

SEQ ID NO: 70 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttccgagggcaggtaataacaatatttctgcatataaatatttagtccaagctaggcccttttgctaatcatgttcatacctcttatcttcctcccacag

SEQ ID NO: 71 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttccgagggcaggtaataacaatatttctgcatataaatatttagtccaagctaggcccttttgctaatcatgttcatacctcttatcttcctcccacag

SEQ ID NO: 72 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcggtgccgaggtaatatcaatatttctgcatataaatatttagtccaagctaggcccttttgctaatcatgttcatacctcttatcttcctcccacag

SEQ ID NO: 73 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcggtgccgaggtaatatcaatatttctgcatataaatatttagtccaagctaggcccttttgctaatcatgttcatacctcttatcttcctcccacag

SEQ ID NO: 74 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcggtgactaggtaataccaatatttctgcatataaatatttagtccaagctaggcccttttgctaatcatgttcatacctcttatcttcctcccacag

SEQ ID NO: 75 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcggtgactaggtaataccaatatttctgcatataaatatttagtccaagctaggcccttttgctaatcatgttcatacctcttatcttcctcccacag

SEQ ID NO: 76 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttccgagggcaggtaatatcaatatttctgcatataaatatttagtccaagctaggcccttttgctaatcatgttcatacctcttatcttcctcccacag

SEQ ID NO: 77 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttccgagggcaggtaatatcaatatttctgcatataaatatttagtccaagctaggcccttttgctaatcatgttcatacctcttatcttcctcccacag

SEQ ID NO: 78 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcagcgcagaggtaataacaatatttctgcatataaatatttagtccaagctaggcccttttgctaatcatgttcatacctcttatcttcctcccacag

SEQ ID NO: 79 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcagcgcagaggtaataacaatatttctgcatataaatatttagtccaagctaggcccttttgctaatcatgttcatacctcttatcttcctcccacag

SEQ ID NO: 80 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcagcgaataggtaagtccaatatttctgcatataaatatttagtccaagctaggcccttttgctaatcatgttcatacctcttatcttcctcccacag

SEQ ID NO: 81 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcagcgaataggtaagtccaatatttctgcatataaatatttagtccaagctaggcccttttgctaatcatgttcatacctcttatcttcctcccacag

SEQ ID NO: 82 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttccgagggcaggtaagtacaatatttctgcatataaatatttagtccaagctaggcccttttgctaatcatgttcatacctcttatcttcctcccacag

SEQ ID NO: 83 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttccgagggcaggtaagtacaatatttctgcatataaatatttagtccaagctaggcccttttgctaatcatgttcatacctcttatcttcctcccacag

SEQ ID NO: 84 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcggtgccgaggtaagttcaatatttctgcatataaatatttagtccaagctaggcccttttgctaatcatgttcatacctcttatcttcctcccacag

SEQ ID NO: 85 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcggtgccgaggtaagttcaatatttctgcatataaatatttagtccaagctaggcccttttgctaatcatgttcatacctcttatcttcctcccacag

SEQ ID NO: 86 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcggtgactaggtaagtccaatatttctgcatataaatatttagtccaagctaggcccttttgctaatcatgttcatacctcttatcttcctcccacag

SEQ ID NO: 87 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcggtgactaggtaagtccaatatttctgcatataaatatttagtccaagctaggcccttttgctaatcatgttcatacctcttatcttcctcccacag

SEQ ID NO: 88 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttccgagggcaggtaagttcaatatttctgcatataaatatttagtccaagctaggcccttttgctaatcatgttcatacctcttatcttcctcccacag

SEQ ID NO: 89 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttccgagggcaggtaagttcaatatttctgcatataaatatttagtccaagctaggcccttttgctaatcatgttcatacctcttatcttcctcccacag

SEQ ID NO: 90 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttcagcgcagaggtaagtacaatatttctgcatataaatatttagtccaagctaggcccttttgctaatcatgttcatacctcttatcttcctcccacag

SEQ ID NO: 91 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttcagcgcagaggtaagtacaatatttctgcatataaatatttagtccaagctaggcccttttgctaatcatgttcatacctcttatcttcctcccacag

SEQ ID NO: 92 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttagtccaagctaggcccttttgctaatcatgttcatacctcttatcttcctcccacag

SEQ ID NO: 93 - DNA, 107 bp, Artificial
  Mutant intron sequence
tctaaatttcagttgacttgtcatcttgatttctggagaccacaaggtaatgaaaaataattacaagagtcttccatctgttgcagtattaaaatggtgagtaagtc

SEQ ID NO: 94 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtaatagagtc

SEQ ID NO: 95 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtaagtgagtc

SEQ ID NO: 96 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtgagtgagtc

SEQ ID NO: 97 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtaagggagtc

SEQ ID NO: 98 - DNA, 107 bp, Artificial; identical to SEQ ID NO: 95
  Mutant intron sequence

SEQ ID NO: 99 - DNA, 107 bp, Artificial
  Mutant intron sequence
tctaaatttcagttgacttgtcatcttgatttctggagaccacaaggtaatgaaaaataattacaagagtcttccatctgttgcagttgccaataggtaagtgagtc

SEQ ID NO: 100 - DNA, 107 bp, Artificial
  Mutant intron sequence
tctaaatttcagttgacttgtcatcttgatttctggagaccacaaggtaatgaaaaataattacaagagtcttccatctgttgcagttgccaataggtaagagagtc

SEQ ID NO: 101 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtaagagagtc

SEQ ID NO: 102 - DNA, 107 bp, Artificial
  Mutant intron sequence
tctaaatttcagttgacttgtcatcttgatttctggagaccacaaggtaatgaaaaataattacaagagtcttccatctgttgcagttgccaataggtaattgagtc

SEQ ID NO: 103 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtaattgagtc

SEQ ID NO: 104 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtgagagagtc

SEQ ID NO: 105 - DNA, 107 bp, Artificial; identical to SEQ ID NO: 101
  Mutant intron sequence

SEQ ID NO: 106 - DNA, 107 bp, Artificial
  Mutant intron sequence
tctaaatttcagttgacttgtcatcttgatttctggagaccacaaggtaatgaaaaataattacaagagtcttccatctgttgcagttgggttaaggtgattgagtc

SEQ ID NO: 107 - DNA, 107 bp, Artificial; identical to SEQ ID NO: 103
  Mutant intron sequence

SEQ ID NO: 108 - DNA, 107 bp, Artificial
  Mutant intron sequence
tctaaatttcagttgacttgtcatcttgatttctggagaccacaaggtaatgaaaaataattacaagagtcttccatctgttgcagtnnnnnnnnngtnnnnnagtc

SEQ ID NO: 109 - DNA, 107 bp, Artificial
  Mutant intron sequence
tctaaatttcagttgacttgtcatcttgatttctggagaccacaaggtaatgaaaaataattacaagagtcttccatctgttgcagtngngnnnaggtaatanagtc

SEQ ID NO: 110 - DNA, 107 bp, Artificial
  Mutant intron sequence
tctaaatttcagttgacttgtcatcttgatttctggagaccacaaggtaatgaaaaataattacaagagtcttccatctgttgcagtngngnnnaggtaagtnagtc

SEQ ID NO: 111 - DNA, 107 bp, Artificial
  Mutant intron sequence
tctaaatttcagttgacttgtcatcttgatttctggagaccacaaggtaatgaaaaataattacaagagtcttccatctgttgcagtnnnnnnaaggtaatagagtc

SEQ ID NO: 112 - DNA, 107 bp, Artificial
  Mutant intron sequence
tctaaatttcagttgacttgtcatcttgatttctggagaccacaaggtaatgaaaaataattacaagagtcttccatctgttgcagtngnnnnnaggtaatagagtc

SEQ ID NO: 113 - DNA, 107 bp, Artificial
  Mutant intron sequence
tctaaatttcagttgacttgtcatcttgatttctggagaccacaaggtaatgaaaaataattacaagagtcttccatctgttgcagtnngnnnnaggtaatagagtc

SEQ ID NO: 114 - DNA, 107 bp, Artificial
  Mutant intron sequence
tctaaatttcagttgacttgtcatcttgatttctggagaccacaaggtaatgaaaaataattacaagagtcttccatctgttgcagtnnngnnnaggtaatagagtc

SEQ ID NO: 115 - DNA, 107 bp, Artificial
  Mutant intron sequence
tctaaatttcagttgacttgtcatcttgatttctggagaccacaaggtaatgaaaaataattacaagagtcttccatctgttgcagtnnnntnnaggtaatagagtc

SEQ ID NO: 116 - DNA, 107 bp, Artificial
  Mutant intron sequence
tctaaatttcagttgacttgtcatcttgatttctggagaccacaaggtaatgaaaaataattacaagagtcttccatctgttgcagtnggnnnnnggtaatagagtc

SEQ ID NO: 117 - DNA, 106 bp, Artificial
  Mutant intron sequence
tctaaatttcagttgacttgtcatcttgatttctggagaccacaaggtaatgaaaaataattacaagagtcttccatctgttgcagtnnggnnnggtaatagagtc

SEQ ID NO: 118 - DNA, 107 bp, Artificial
  Mutant intron sequence
tctaaatttcagttgacttgtcatcttgatttctggagaccacaaggtaatgaaaaataattacaagagtcttccatctgttgcagtnnngtnnnggtaatagagtc

SEQ ID NO: 119 - DNA, 107 bp, Artificial
  Mutant intron sequence
tctaaatttcagttgacttgtcatcttgatttctggagaccacaaggtaatgaaaaataattacaagagtcttccatctgttgcagtnnnnttnnggtaatagagtc

SEQ ID NO: 120 - DNA, 107 bp, Artificial
  Mutant intron sequence
tctaaatttcagttgacttgtcatcttgatttctggagaccacaaggtaatgaaaaataattacaagagtcttccatctgttgcagtnnnnntanggtaatagagtc

SEQ ID NO: 121 - DNA, 107 bp, Artificial
  Mutant intron sequence
tctaaatttcagttgacttgtcatcttgatttctggagaccacaaggtaatgaaaaataattacaagagtcttccatctgttgcagtnnnnnnaaggtaagtgagtc

SEQ ID NO: 122 - DNA, 107 bp, Artificial
  Mutant intron sequence
tctaaatttcagttgacttgtcatcttgatttctggagaccacaaggtaatgaaaaataattacaagagtcttccatctgttgcagtngnnnnnaggtaagtgagtc

SEQ ID NO: 123 - DNA, 107 bp, Artificial
  Mutant intron sequence
tctaaatttcagttgacttgtcatcttgatttctggagaccacaaggtaatgaaaaataattacaagagtcttccatctgttgcagtnngnnnnaggtaagtgagtc

SEQ ID NO: 124 - DNA, 107 bp, Artificial
  Mutant intron sequence
tctaaatttcagttgacttgtcatcttgatttctggagaccacaaggtaatgaaaaataattacaagagtcttccatctgttgcagtnnngnnnaggtaagtgagtc

SEQ ID NO: 125 - DNA, 107 bp, Artificial
  Mutant intron sequence
tctaaatttcagttgacttgtcatcttgatttctggagaccacaaggtaatgaaaaataattacaagagtcttccatctgttgcagtnnnntnnaggtaagtgagtc

SEQ ID NO: 126 - DNA, 107 bp, Artificial
  Mutant intron sequence
tctaaatttcagttgacttgtcatcttgatttctggagaccacaaggtaatgaaaaataattacaagagtcttccatctgttgcagtnggnnnnnggtaagtgagtc

SEQ ID NO: 127 - DNA, 107 bp, Artificial
  Mutant intron sequence
tctaaatttcagttgacttgtcatcttgatttctggagaccacaaggtaatgaaaaataattacaagagtcttccatctgttgcagtnnggnnnnggtaagtgagtc

SEQ ID NO: 128 - DNA, 107 bp, Artificial
  Mutant intron sequence
tctaaatttcagttgacttgtcatcttgatttctggagaccacaaggtaatgaaaaataattacaagagtcttccatctgttgcagtnnngtnnnggtaagtgagtc

SEQ ID NO: 129 - DNA, 107 bp, Artificial
  Mutant intron sequence
tctaaatttcagttgacttgtcatcttgatttctggagaccacaaggtaatgaaaaataattacaagagtcttccatctgttgcagtnnnnttnnggtaagtgagtc

SEQ ID NO: 130 - DNA, 107 bp, Artificial
  Mutant intron sequence
tctaaatttcagttgacttgtcatcttgatttctggagaccacaaggtaatgaaaaataattacaagagtcttccatctgttgcagtnnnnntanggtaagtgagtc

SEQ ID NO: 131 - DNA, 107 bp, Artificial
  Mutant intron sequence
tctaaatttcagttgacttgtcatcttgatttctggagaccacaaggtaatgaaaaataattacaagagtcttccatctgttgcagtagcgaataggtaatacagtc

SEQ ID NO: 132 - DNA, 107 bp, Artificial
  Mutant intron sequence
tctaaatttcagttgacttgtcatcttgatttctggagaccacaaggtaatgaaaaataattacaagagtcttccatctgttgcagtcgagggcaggtaataaagtc

SEQ ID NO: 133 - DNA, 107 bp, Artificial
  Mutant intron sequence
tctaaatttcagttgacttgtcatcttgatttctggagaccacaaggtaatgaaaaataattacaagagtcttccatctgttgcagtggtgccgaggtaatatagtc

SEQ ID NO: 134 - DNA, 107 bp, Artificial
  Mutant intron sequence
tctaaatttcagttgacttgtcatcttgatttctggagaccacaaggtaatgaaaaataattacaagagtcttccatctgttgcagtggtgactaggtaatacagtc

SEQ ID NO: 135 - DNA, 107 bp, Artificial
  Mutant intron sequence
tctaaatttcagttgacttgtcatcttgatttctggagaccacaaggtaatgaaaaataattacaagagtcttccatctgttgcagtcgagggcaggtaatatagtc

SEQ ID NO: 136 - DNA, 107 bp, Artificial
  Mutant intron sequence
tctaaatttcagttgacttgtcatcttgatttctggagaccacaaggtaatgaaaaataattacaagagtcttccatctgttgcagtagcgcacacctaataaagtc

SEQ ID NO: 137 - DNA, 107 bp, Artificial
  Mutant intron sequence
tctaaatttcagttgacttgtcatcttgatttctggagaccacaaggtaatgaaaaataattacaagagtcttccatctgttgcagtagcgaataggtaagtcagtc

SEQ ID NO: 138 - DNA, 107 bp, Artificial
  Mutant intron sequence
tctaaatttcagttgacttgtcatcttgatttctggagaccacaaggtaatgaaaaataattacaagagtcttccatctgttgcagtcgagggcaggtaagtaagtc

SEQ ID NO: 139 - DNA, 107 bp, Artificial
  Mutant intron sequence
tctaaatttcagttgacttgtcatcttgatttctggagaccacaaggtaatgaaaaataattacaagagtcttccatctgttgcagtggtgccgaggtaagttagtc

SEQ ID NO: 140 - DNA, 107 bp, Artificial
  Mutant intron sequence
tctaaatttcagttgacttgtcatcttgatttctggagaccacaaggtaatgaaaaataattacaagagtcttccatctgttgcagtggtgactaggtaagtcagtc

SEQ ID NO: 141 - DNA, 107 bp, Artificial
  Mutant intron sequence
tctaaatttcagttgacttgtcatcttgatttctggagaccacaaggtaatgaaaaataattacaagagtcttccatctgttgcagtcgagggcaggtaagttagtc

SEQ ID NO: 142 - DNA, 107 bp, Artificial
  Mutant intron sequence
tctaaatttcagttgacttgtcatcttgatttctggagaccacaaggtaatgaaaaataattacaagagtcttccatctgttgcagtagcgcagaggtaagtaagtc

SEQ ID NO: 143 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtgagagcaatatttctgcatataaatatttagtccaagctaggcccttttgctaatcatgttcatacctcttatcttcctcccacag

SEQ ID NO: 144 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtgagagcaatatttctgcatataaatatttagtccaagctaggcccttttgctaatcatgttcatacctcttatcttcctcccacag

SEQ ID NO: 145 - DNA, 247 bp, Artificial
  Mutant intron sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtgattgcaatatttctgcatataaatatttagtccaagctaggcccttttgctaatcatgttcatacctcttatcttcctcccacag

SEQ ID NO: 146 - DNA, 247 bp, Artificial
  Mutant intron sequence
gtgagtctatgggacccttgatgttcttttaatatacttttttgtttatcttatttctaatactttccctcttctctttctttcagggcaataatgatacaatgtatcatgcctctttgcaccattctaaagaataacagtgataatttctgggttaaggtgattgcaatatttctgcatataaatatttagtccaagctaggcccttttgctaatcatgttcatacctcttatcttcctcccacag

SEQ ID NO: 147 - DNA, 247 bp, Artificial; identical to SEQ ID NO: 10
  Mutant intron sequence

SEQ ID NO: 148 - DNA, 247 bp, Artificial; identical to SEQ ID NO: 11
  Mutant intron sequence

SEQ ID NO: 149 - DNA, 247 bp, Artificial; identical to SEQ ID NO: 12
  Mutant intron sequence

SEQ ID NO: 150 - DNA, 247 bp, Artificial; identical to SEQ ID NO: 13
  Mutant intron sequence

SEQ ID NO: 151 - DNA, 850 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgccaataggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 152 - DNA, 850 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgccaataggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 153 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtgagtgcaatatttctgcatataaatatttagtccaagctaggcccttttgctaatcatgttcatacctcttatcttcctcccacag

SEQ ID NO: 154 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtgagtgcaatatttctgcatataaatatttagtccaagctaggcccttttgctaatcatgttcatacctcttatcttcctcccacag

SEQ ID NO: 155 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagggcaatatttctgcatataaatatttagtccaagctaggcccttttgctaatcatgttcatacctcttatcttcctcccacag

SEQ ID NO: 156 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaagggcaatatttctgcatataaatatttagtccaagctaggcccttttgctaatcatgttcatacctcttatcttcctcccacag

SEQ ID NO: 157 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtgagagcaatatttctgcatataaatatttagtccaagctaggcccttttgctaatcatgttcatacctcttatcttcctcccacag

SEQ ID NO: 158 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtgagagcaatatttctgcatataaatatttagtccaagctaggcccttttgctaatcatgttcatacctcttatcttcctcccacag

SEQ ID NO: 159 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtgattgcaatatttctgcatataaatatttagtccaagctaggcccttttgctaatcatgttcatacctcttatcttcctcccacag

SEQ ID NO: 160 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtgattgcaatatttctgcatataaatatttagtccaagctaggcccttttgctaatcatgttcatacctcttatcttcctcccacag

SEQ ID NO: 161 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagagcaatatttctgcatataaatatttagtccaagctaggcccttttgctaatcatgttcatacctcttatcttcctcccacag

SEQ ID NO: 162 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaagagcaatatttctgcatataaatatttagtccaagctaggcccttttgctaatcatgttcatacctcttatcttcctcccacag

SEQ ID NO: 163 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaattgcaatatttctgcatataaatatttagtccaagctaggcccttttgctaatcatgttcatacctcttatcttcctcccacag

SEQ ID NO: 164 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaattgcaatatttctgcatataaatatttagtccaagctaggcccttttgctaatcatgttcatacctcttatcttcctcccacag

SEQ ID NO: 165 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgccaataggtgagtgcaatatttctgcatataaatatttagtccaagctaggcccttttgctaatcatgttcatacctcttatcttcctcccacag

SEQ ID NO: 166 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgccaataggtaagggcaatatttctgcatataaatatttagtccaagctaggcccttttgctaatcatgttcatacctcttatcttcctcccacag

SEQ ID NO: 167 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgccaataggtgagtgcaatatttctgcatataaatatttagtccaagctaggcccttttgctaatcatgttcatacctcttatcttcctcccacag

SEQ ID NO: 168 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgccaataggtaagggcaatatttctgcatataaatatttagtccaagctaggcccttttgctaatcatgttcatacctcttatcttcctcccacag

SEQ ID NO: 169 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgccaataggtaagagcaatatttctgcatataaatatttagtccaagctaggcccttttgctaatcatgttcatacctcttatcttcctcccacag

SEQ ID NO: 170 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgccaataggtaattgcaatatttctgcatataaatatttagtccaagctaggcccttttgctaatcatgttcatacctcttatcttcctcccacag

SEQ ID NO: 171 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgccaataggtaagagcaatatttctgcatataaatatttagtccaagctaggcccttttgctaatcatgttcatacctcttatcttcctcccacag

SEQ ID NO: 172 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgccaataggtaattgcaatatttctgcatataaatatttagtccaagctaggcccttttgctaatcatgttcatacctcttatcttcctcccacag

SEQ ID NO: 173 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnnnnnngtnnnnncaatatttctgcatataaatatttagtccaagctaggcccttttgctaatcatgttcatacctcttatcttcctcccacag

SEQ ID NO: 174 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnnnnnngtnnnnncaatatttctgcatataaatatttagtccaagctaggcccttttgctaatcatgttcatacctcttatcttcctcccacag

SEQ ID NO: 175 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcngngnnnaggtaatancaatatttctgcatataaatatttagtccaagctaggcccttttgctaatcatgttcatacctcttatcttcctcccacag

SEQ ID NO: 176 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcngngnnnaggtaatancaatatttctgcatataaatatttagtccaagctaggcccttttgctaatcatgttcatacctcttatcttcctcccacag

SEQ ID NO: 177 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcngngnnnaggtaagtncaatatttctgcatataaatatttagtccaagctaggcccttttgctaatcatgttcatacctcttatcttcctcccacag

SEQ ID NO: 178 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcngngnnnaggtaagtncaatatttctgcatataaatatttagtccaagctaggcccttttgctaatcatgttcatacctcttatcttcctcccacag

SEQ ID NO: 179 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnnnaaggtaatagcaatatttctgcatataaatatttagtccaagctaggcccttttgctaatcatgttcatacctcttatcttcctcccacag

SEQ ID NO: 180 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnnnaaggtaatagcaatatttctgcatataaatatttagtccaagctaggcccttttgctaatcatgttcatacctcttatcttcctcccacag

SEQ ID NO: 181 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcngnnnnnaggtaatagcaatatttctgcatataaatatttagtccaagctaggcccttttgctaatcatgttcatacctcttatcttcctcccacag

SEQ ID NO: 182 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcngnnnnnaggtaatagcaatatttctgcatataaatatttagtccaagctaggcccttttgctaatcatgttcatacctcttatcttcctcccacag

SEQ ID NO: 183 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnngnnnnaggtaatagcaatatttctgcatataaatatttagtccaagctaggcccttttgctaatcatgttcatacctcttatcttcctcccacag

SEQ ID NO: 184 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnngnnnnaggtaatagcaatatttctgcatataaatatttagtccaagctaggcccttttgctaatcatgttcatacctcttatcttcctcccacag

SEQ ID NO: 185 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnngnnnaggtaatagcaatatttctgcatataaatatttagtccaagctaggcccttttgctaatcatgttcatacctcttatcttcctcccacag

SEQ ID NO: 186 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnngnnnaggtaatagcaatatttctgcatataaatatttagtccaagctaggcccttttgctaatcatgttcatacctcttatcttcctcccacag

SEQ ID NO: 187 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnntnnaggtaatagcaatatttctgcatataaatatttagtccaagctaggcccttttgctaatcatgttcatacctcttatcttcctcccacag

SEQ ID NO: 188 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnntnnaggtaatagcaatatttctgcatataaatatttagtccaagctaggcccttttgctaatcatgttcatacctcttatcttcctcccacag

SEQ ID NO: 189 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnggnnnnnggtaatagcaatatttctgcatataaatatttagtccaagctaggcccttttgctaatcatgttcatacctcttatcttcctcccacag

SEQ ID NO: 190 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnggnnnnnggtaatagcaatatttctgcatataaatatttagtccaagctaggcccttttgctaatcatgttcatacctcttatcttcctcccacag

SEQ ID NO: 191 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnggnnnnggtaatagcaatatttctgcatataaatatttagtccaagctaggcccttttgctaatcatgttcatacctcttatcttcctcccacag

SEQ ID NO: 192 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnggnnnnggtaatagcaatatttctgcatataaatatttagtccaagctaggcccttttgctaatcatgttcatacctcttatcttcctcccacag

SEQ ID NO: 193 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnngtnnnggtaatagcaatatttctgcatataaatatttagtccaagctaggcccttttgctaatcatgttcatacctcttatcttcctcccacag

SEQ ID NO: 194 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnngtnnnggtaatagcaatatttctgcatataaatatttagtccaagctaggcccttttgctaatcatgttcatacctcttatcttcctcccacag

SEQ ID NO: 195 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnttnnggtaatagcaatatttctgcatataaatatttagtccaagctaggcccttttgctaatcatgttcatacctcttatcttcctcccacag

SEQ ID NO: 196 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnttnnggtaatagcaatatttctgcatataaatatttagtccaagctaggcccttttgctaatcatgttcatacctcttatcttcctcccacag

SEQ ID NO: 197 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnntanggtaatagcaatatttctgcatataaatatttagtccaagctaggcccttttgctaatcatgttcatacctcttatcttcctcccacag

SEQ ID NO: 198 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnntanggtaatagcaatatttctgcatataaatatttagtccaagctaggcccttttgctaatcatgttcatacctcttatcttcctcccacag

SEQ ID NO: 199 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnnnaaggtaagtgcaatatttctgcatataaatatttagtccaagctaggcccttttgctaatcatgttcatacctcttatcttcctcccacag

SEQ ID NO: 200 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnnnaaggtaagtgcaatatttctgcatataaatatttagtccaagctaggcccttttgctaatcatgttcatacctcttatcttcctcccacag

SEQ ID NO: 201 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcngnnnnnaggtaagtgcaatatttctgcatataaatatttagtccaagctaggcccttttgctaatcatgttcatacctcttatcttcctcccacag

SEQ ID NO: 202 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcngnnnnnaggtaagtgcaatatttctgcatataaatatttagtccaagctaggcccttttgctaatcatgttcatacctcttatcttcctcccacag

SEQ ID NO: 203 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnngnnnnaggtaagtgcaatatttctgcatataaatatttagtccaagctaggcccttttgctaatcatgttcatacctcttatcttcctcccacag

SEQ ID NO: 204 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnngnnnnaggtaagtgcaatatttctgcatataaatatttagtccaagctaggcccttttgctaatcatgttcatacctcttatcttcctcccacag

SEQ ID NO: 205 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnngnnnaggtaagtgcaatatttctgcatataaatatttagtccaagctaggcccttttgctaatcatgttcatacctcttatcttcctcccacag

SEQ ID NO: 206 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnngnnnaggtaagtgcaatatttctgcatataaatatttagtccaagctaggcccttttgctaatcatgttcatacctcttatcttcctcccacag

SEQ ID NO: 207 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnntnnaggtaagtgcaatatttctgcatataaatatttagtccaagctaggcccttttgctaatcatgttcatacctcttatcttcctcccacag

SEQ ID NO: 208 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnntnnaggtaagtgcaatatttctgcatataaatatttagtccaagctaggcccttttgctaatcatgttcatacctcttatcttcctcccacag

SEQ ID NO: 209 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnggnnnnnggtaagtgcaatatttctgcatataaatatttagtccaagctaggcccttttgctaatcatgttcatacctcttatcttcctcccacag

SEQ ID NO: 210 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnggnnnnnggtaagtgcaatatttctgcatataaatatttagtccaagctaggcccttttgctaatcatgttcatacctcttatcttcctcccacag

SEQ ID NO: 211 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnggnnnnggtaagtgcaatatttctgcatataaatatttagtccaagctaggcccttttgctaatcatgttcatacctcttatcttcctcccacag

SEQ ID NO: 212 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnggnnnnggtaagtgcaatatttctgcatataaatatttagtccaagctaggcccttttgctaatcatgttcatacctcttatcttcctcccacag

SEQ ID NO: 213 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnngtnnnggtaagtgcaatatttctgcatataaatatttagtccaagctaggcccttttgctaatcatgttcatacctcttatcttcctcccacag

SEQ ID NO: 214 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnngtnnnggtaagtgcaatatttctgcatataaatatttagtccaagctaggcccttttgctaatcatgttcatacctcttatcttcctcccacag

SEQ ID NO: 215 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnttnnggtaagtgcaatatttctgcatataaatatttagtccaagctaggcccttttgctaatcatgttcatacctcttatcttcctcccacag

SEQ ID NO: 216 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnttnnggtaagtgcaatatttctgcatataaatatttagtccaagctaggcccttttgctaatcatgttcatacctcttatcttcctcccacag

SEQ ID NO: 217 - DNA, 741 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcnnnnntanggtaatgtgcaatatttctgcatataaatatttagtccaagctaggcccttttgctaatcatgttcatacctcttatcttcctcccacag

SEQ ID NO: 218 - DNA, 741 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcnnnnntanggtaatgtgcaatatttctgcatataaatatttagtccaagctaggcccttttgctaatcatgttcatacctcttatcttcctcccacag

SEQ ID NO: 219 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcagcgaataggtaataccaatatttctgcatataaatatttagtccaagctaggcccttttgctaatcatgttcatacctcttatcttcctcccacag

SEQ ID NO: 220 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcagcgaataggtaataccaatatttctgcatataaatatttagtccaagctaggcccttttgctaatcatgttcatacctcttatcttcctcccacag

SEQ ID NO: 221 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttccgagggcaggtaataacaatatttctgcatataaatatttagtccaagctaggcccttttgctaatcatgttcatacctcttatcttcctcccacag

SEQ ID NO: 222 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttccgagggcaggtaataacaatatttctgcatataaatatttagtccaagctaggcccttttgctaatcatgttcatacctcttatcttcctcccacag

SEQ ID NO: 223 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcggtgccgaggtaatatcaatatttctgcatataaatatttagtccaagctaggcccttttgctaatcatgttcatacctcttatcttcctcccacag

SEQ ID NO: 224 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcggtgccgaggtaatatcaatatttctgcatataaatatttagtccaagctaggcccttttgctaatcatgttcatacctcttatcttcctcccacag

SEQ ID NO: 225 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcggtgactaggtaataccaatatttctgcatataaatatttagtccaagctaggcccttttgctaatcatgttcatacctcttatcttcctcccacag

SEQ ID NO: 226 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcggtgactaggtaataccaatatttctgcatataaatatttagtccaagctaggcccttttgctaatcatgttcatacctcttatcttcctcccacag

SEQ ID NO: 227 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttccgagggcaggtaatatcaatatttctgcatataaatatttagtccaagctaggcccttttgctaatcatgttcatacctcttatcttcctcccacag

SEQ ID NO: 228 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttccgagggcaggtaatatcaatatttctgcatataaatatttagtccaagctaggcccttttgctaatcatgttcatacctcttatcttcctcccacag

SEQ ID NO: 229 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcagcgcagaggtaataacaatatttctgcatataaatatttagtccaagctaggcccttttgctaatcatgttcatacctcttatcttcctcccacag

SEQ ID NO: 230 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcagcgcagaggtaataacaatatttctgcatataaatatttagtccaagctaggcccttttgctaatcatgttcatacctcttatcttcctcccacag

SEQ ID NO: 231 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcagcgaataggtaagtccaatatttctgcatataaatatttagtccaagctaggcccttttgctaatcatgttcatacctcttatcttcctcccacag

SEQ ID NO: 232 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcagcgaataggtaagtccaatatttctgcatataaatatttagtccaagctaggcccttttgctaatcatgttcatacctcttatcttcctcccacag

SEQ ID NO: 233 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttccgagggcaggtaagtacaatatttctgcatataaatatttagtccaagctaggcccttttgctaatcatgttcatacctcttatcttcctcccacag

SEQ ID NO: 234 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttccgagggcaggtaagtacaatatttctgcatataaatatttagtccaagctaggcccttttgctaatcatgttcatacctcttatcttcctcccacag

SEQ ID NO: 235 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcggtgccgaggtaagttcaatatttctgcatataaatatttagtccaagctaggcccttttgctaatcatgttcatacctcttatcttcctcccacag

SEQ ID NO: 236 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcggtgccgaggtaagttcaatatttctgcatataaatatttagtccaagctaggcccttttgctaatcatgttcatacctcttatcttcctcccacag

SEQ ID NO: 237 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcggtgactaggtaagtccaatatttctgcatataaatatttagtccaagctaggcccttttgctaatcatgttcatacctcttatcttcctcccacag

SEQ ID NO: 238 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcggtgactaggtaagtccaatatttctgcatataaatatttagtccaagctaggcccttttgctaatcatgttcatacctcttatcttcctcccacag

SEQ ID NO: 239 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttccgagggcaggtaagttcaatatttctgcatataaatatttagtccaagctaggcccttttgctaatcatgttcatacctcttatcttcctcccacag

SEQ ID NO: 240 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttccgagggcaggtaagttcaatatttctgcatataaatatttagtccaagctaggcccttttgctaatcatgttcatacctcttatcttcctcccacag

SEQ ID NO: 241 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttcagcgcagaggtaagtacaatatttctgcatataaatatttagtccaagctaggcccttttgctaatcatgttcatacctcttatcttcctcccacag

SEQ ID NO: 242 - DNA, 740 bp, Artificial
  Mutant intron sequence
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttcagcgcagaggtaagtacaatatttctgcatataaatatttagtccaagctaggcccttttgctaatcatgttcatacctcttatcttcctcccacag

SEQ ID NO: 243 - DNA, 7713 bp, Artificial
  Plasmid TRCBA-int-luc mut (654 C-T)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 244 - DNA, 7713 bp, Artificial
  Plasmid TRCBA-int-luc (wt)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 245 - DNA, 7713 bp, Artificial
  Plasmid TRCBA-int-luc (654 C-T, 657 TA-GT)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 246 - DNA, 5860 bp, Artificial
  Plasmid GL3-int-Luc mut (654 C-T)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 247 - DNA, 5860 bp, Artificial
  Plasmid GL3-int-Luc (wt)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 248 - DNA, 5860 bp, Artificial
  Plasmid GL3-int-Luc (654 C-T, 657 TA-GT)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 249 - DNA, 6683 bp, Artificial
  Plasmid GL3-2int-fron-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 250 - DNA, 7547 bp, Artificial
  Plasmid GL3-3int-2fron-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 251 - DNA, 5860 bp, Artificial
  Plasmid GL3-int-luc A (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 252 - DNA, 5860 bp, Artificial
  Plasmid GL3-int-Luc B
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 253 - DNA, 5860 bp, Artificial
  Plasmid GL3-int-Luc C
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 254 - DNA, 5833 bp, Artificial
  Plasmid GL3-int-fron (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 255 - DNA, 6710 bp, Artificial
  Plasmid GL3-2int-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 256 - DNA, 6710 bp, Artificial
  Plasmid GL3-2int-Sph-C
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 257 - DNA, 5660 bp, Artificial
  Plasmid GL3-sint200-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 258 - DNA, 5660 bp, Artificial
  Plasmid GL3-sint200-sph (657 GT)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 259 - DNA, 5436 bp, Artificial
  Plasmid GL3-sint425-sph
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 260 - DNA, 7449 bp, Artificial
  Plasmid TRCBA with antitrypsin and mutant beta-globin intron (654C-T)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttggggattttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtaaccccccccccccctgcaggggccctcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 261 - DNA, 850 bp, Artificial
  Mutant beta globin intron (654C-T)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 262 - DNA, 850 bp, Homo sapiens
  misc_feature (1)..(850): Wild-type beta-globin intron
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 263 - DNA, 850 bp, Artificial
  Double mutant beta globin intron (654C-T 657TA-GT)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 264 - DNA, 2503 bp, Artificial
  Luciferase with mutant beta-globin intron (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 265 - DNA, 2503 bp, Artificial
  Luciferase with wild-type beta-globin intron
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 266 - DNA, 2503 bp, Artificial
  Luciferase with double mutant beta-globin intron (654C-T 657TA-GT)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 267 - DNA, 3355 bp, Artificial
  Luciferase with mutant beta-globin intron (654C-T) with mutant beta-globin intron (654C-T) upstream to translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 268 - DNA, 4219 bp, Artificial
  Luciferaase with mutant beta-globin intron (654C-T) and two mutant beta-globin introns (654C-T) upstream to translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 269 - DNA, 2503 bp, Artificial
  Luciferase with mutant beta-globin intron (654C-T) alternative location A
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 270 - DNA, 2503 bp, Artificial
  Luciferase with mutant beta-globin intron (654C-T) alternative location B
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 271 - DNA, 2503 bp, Artificial
  Luciferase with mutant beta-globin intron (654C-T) alternative location C
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 272 - DNA, 2505 bp, Artificial
  Luciferase with mutant beta-globin intron upstream of translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 273 - DNA, 3353 bp, Artificial
  Luciferase with two mutant beta-globin introns (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 274 - DNA, 3353 bp, Artificial
  Luciferase with two mutant beta-globin introns (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 275 - DNA, 2303 bp, Artificial
  Luciferase with mutant beta-globin intron (654C-T and 200 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 276 - DNA, 2303 bp, Artificial
  Luciferase with double mutant beta-globin intron (654C-T 657TA-GT and 200 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 277 - DNA, 2079 bp, Artificial
  Luciferase with mutant beta-globin intron (654C-T and 425 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 278 - DNA, 2107 bp, Artificial
  Antitrypsin with mutant beta-globin intron (654C-T)
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataa

SEQ ID NO: 279 - DNA, 18 bp, Artificial
  Synthetic oligonucleotide
gctattaccttaacccag

SEQ ID NO: 280 - DNA, 18 bp, Artificial
  Synthetic oligonucleotide
gcacttaccttaacccag

SEQ ID NO: 281 - DNA, 18 bp, Artificial
  Synthetic oligonucleotide
caagggtcccatagtctc

SEQ ID NO: 282 - DNA, 18 bp, Artificial
  Synthetic oligonucleotide
gaaagagatgagggaaag

SEQ ID NO: 283 - DNA, 18 bp, Artificial
  Synthetic oligonucleotide
gaaagagaagagggaaag

SEQ ID NO: 284 - DNA, 18 bp, Artificial
  Synthetic oligonucleotide
cctcttacctcagttaca

SEQ ID NO: 285 - DNA, 18 bp, Artificial
  Synthetic oligonucleotide
ctgtgggagtaagataag

SEQ ID NO: 286 - DNA, 18 bp, Artificial
  Synthetic oligonucleotide
gctcttaccttaacccag

SEQ ID NO: 287 - DNA, 18 bp, Artificial
  Synthetic oligonucleotide
gcaattaccttaacccag

SEQ ID NO: 288 - DNA, 18 bp, Artificial
  Synthetic oligonucleotide
caagggtcccatagactc

SEQ ID NO: 289 - DNA, 18 bp, Artificial
  Synthetic oligonucleotide
gaaagagattagggaaag

SEQ ID NO: 290 - DNA, 18 bp, Artificial
  Synthetic oligonucleotide
ctgtgggaggaagataag

SEQ ID NO: 291 - DNA, 18 bp, Artificial
  Synthetic oligonucleotide
cctcttacatcagttaca

SEQ ID NO: 292 - DNA, 850 bp, Artificial
  IVS2-654 intron with 564CT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 293 - DNA, 850 bp, Artificial
  IVS2-654 intron with 657G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 294 - DNA, 850 bp, Artificial
  IVS2-654 intron with 658T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaattgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 295 - DNA, 850 bp, Artificial; identical to SEQ ID NO: 263
  IVS2-654 intron with 657GT mutation

SEQ ID NO: 296 - DNA, 650 bp, Artificial
  IVS2-654 intron with 200 bp deletion
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 297 - DNA, 426 bp, Artificial
  IVS2-654 intron with 425 bp deletion
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 298 - DNA, 196 bp, Artificial
  IVS2-654 intron 197 bp
gtgagtctatgggacccttgatgttcttttaatatacttttttgtttatcttatttctaatactttccctcttctctttctttcaggtgattgactgactgggttaaggtaatagcgccgttgaaaacctcagccgtatagtccaagctaggcccttttgctaatcatgttcatacctcttatcttcctcccacag

SEQ ID NO: 299 - DNA, 247 bp, Artificial; identical to SEQ ID NO: 92
  IVS-654 intron 247 bp

SEQ ID NO: 300 - DNA, 850 bp, Artificial
  IVS2-654 intron with 6A mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 301 - DNA, 850 bp, Artificial
  IVS2-654 intron with 564C mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 302 - DNA, 850 bp, Artificial
  IVS2-654 intron with 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 303 - DNA, 850 bp, Artificial
  Mutant beta globin intron (705T-G)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 304 - DNA, 850 bp, Artificial
  IVS2-705 intron with 564 CT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 305 - DNA, 850 bp, Artificial
  IVS2-705 intron with 657G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 306 - DNA, 850 bp, Artificial
  IVS2-705 intron with 658T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaattgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 307 - DNA, 850 bp, Artificial
  IVS2-705 intron with 657GT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 308 - DNA, 650 bp, Artificial
  IVS2-705 intron with 200 bp deletion
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 309 - DNA, 426 bp, Artificial
  IVS2-705 intron with 425 bp deletion
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 310 - DNA, 850 bp, Artificial
  IVS2-705 intron with 6A mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 311 - DNA, 850 bp, Artificial
  IVS2-705 intron with 564C mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 312 - DNA, 850 bp, Artificial
  IVS2-705 intron with 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 313 - DNA, 14667 bp, Homo sapiens
  misc_feature (1)..(14667): CFTR gene exon 19
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 314 - DNA, 14667 bp, Homo sapiens
  misc_feature (1)..(14667): CFTR exon 19 containing 3849 + 10 kb C-to-T mutation
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggtgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 315 - DNA, 3733 bp, Mus musculus
  misc_feature (1)..(3733): wild-type Mus musculus dystrophin intron 22, exon 23 and intron 23 sequences
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 316 - DNA, 3733 bp, Mus musculus
  misc_feature (1)..(3733): mdx Mus musculus dystrophin intron 22, exon 23 and intron 23 sequences
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaataaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 317 - DNA, 18 bp, Artificial
  Synthetic oligonucleotide
gtcttactcgccatttta

SEQ ID NO: 318 - DNA, 18 bp, Artificial
  Synthetic oligonucleotide
gtcttactcaccatttta

SEQ ID NO: 319 - DNA, 25 bp, Artificial
  Synthetic oligonucleotide
aacctcggcttacctgaaattttcg

SEQ ID NO: 320 - DNA, 1653 bp, Hotaria parvula
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 321 - DNA, 17578 bp, Homo sapiens
  Intron (1)..(13645): intron 9
gtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccag

SEQ ID NO: 322 - DNA, 18 bp, Artificial
  Synthetic oligonucleotide
tatctgcacctttggtag

SEQ ID NO: 323 - DNA, 21 bp, Artificial
  Synthetic oligonucleotide
tgaaggtactcacactgccgc

SEQ ID NO: 324 - DNA, 16 bp, Artificial
  Mutant intron sequence
nnnnnnnnngtnnnnn

SEQ ID NO: 325 - DNA, 16 bp, Artificial
  Mutant intron sequence
ngngnnnaggtaatan

SEQ ID NO: 326 - DNA, 16 bp, Artificial
  Mutant intron sequence
ngngnnnaggtaagtn

SEQ ID NO: 327 - DNA, 16 bp, Artificial
  Mutant intron sequence
nnnnnnaaggtaatag

SEQ ID NO: 328 - DNA, 16 bp, Artificial
  Mutant intron sequence
ngnnnnnaggtaatag

SEQ ID NO: 329 - DNA, 16 bp, Artificial
  Mutant intron sequence
nngnnnnaggtaatag

SEQ ID NO: 330 - DNA, 16 bp, Artificial
  Mutant intron sequence
nnngnnnaggtaatag

SEQ ID NO: 331 - DNA, 16 bp, Artificial
  Mutant intron sequence
nnnntnnaggtaatag

SEQ ID NO: 332 - DNA, 16 bp, Artificial
  Mutant intron sequence
nggnnnnnggtaatag

SEQ ID NO: 333 - DNA, 16 bp, Artificial
  Mutant intron sequence
nnggnnnnggtaatag

SEQ ID NO: 334 - DNA, 16 bp, Artificial
  Mutant intron sequence
nnngtnnnggtaatag

SEQ ID NO: 335 - DNA, 16 bp, Artificial
  Mutant intron sequence
nnnnttnnggtaatag

SEQ ID NO: 336 - DNA, 16 bp, Artificial
  Mutant intron sequence
nnnnntanggtaatag

SEQ ID NO: 337 - DNA, 16 bp, Artificial
  Mutant intron sequence
nnnnnnaaggtaagtg

SEQ ID NO: 338 - DNA, 16 bp, Artificial
  Mutant intron sequence
ngnnnnnaggtaagtg

SEQ ID NO: 339 - DNA, 16 bp, Artificial
  Mutant intron sequence
nngnnnnaggtaagtg

SEQ ID NO: 340 - DNA, 16 bp, Artificial
  Mutant intron sequence
nnngnnnaggtaagtg

SEQ ID NO: 341 - DNA, 16 bp, Artificial
  Mutant intron sequence
nnnntnnaggtaagtg

SEQ ID NO: 342 - DNA, 16 bp, Artificial
  Mutant intron sequence
nggnnnnnggtaattg

SEQ ID NO: 343 - DNA, 16 bp, Artificial
  Mutant intron sequence
nnggnnnnggtaagtg

SEQ ID NO: 344 - DNA, 16 bp, Artificial
  Mutant intron sequence
nnngtnnnggtaagtg

SEQ ID NO: 345 - DNA, 16 bp, Artificial
  Mutant intron sequence
nnnnttnnggtaagtg

SEQ ID NO: 346 - DNA, 17 bp, Artificial
  Mutant intron sequence
nnnnntanggtaatgtg

SEQ ID NO: 347 - DNA, 16 bp, Artificial
  Mutant intron sequence
agcgaataggtaatac

SEQ ID NO: 348 - DNA, 16 bp, Artificial
  Mutant intron sequence
cgagggcaggtaataa

SEQ ID NO: 349 - DNA, 16 bp, Artificial
  Mutant intron sequence
ggtgccgaggtaatat

SEQ ID NO: 350 - DNA, 16 bp, Artificial
  Mutant intron sequence
ggtgactaggtaatac

SEQ ID NO: 351 - DNA, 16 bp, Artificial
  Mutant intron sequence
cgagggcaggtaatat

SEQ ID NO: 352 - DNA, 16 bp, Artificial
  Mutant intron sequence
agcgcagaggtaataa

SEQ ID NO: 353 - DNA, 16 bp, Artificial
  Mutant intron sequence
agcgaataggtaagtc

SEQ ID NO: 354 - DNA, 16 bp, Artificial
  Mutant intron sequence
cgagggcaggtaagta

SEQ ID NO: 355 - DNA, 16 bp, Artificial
  Mutant intron sequence
ggtgccgaggtaagtt

SEQ ID NO: 356 - DNA, 16 bp, Artificial
  Mutant intron sequence
ggtgactaggtaagtc

SEQ ID NO: 357 - DNA, 16 bp, Artificial
  Mutant intron sequence
cgagggcaggtaagtt

SEQ ID NO: 358 - DNA, 16 bp, Artificial
  Mutant intron sequence
agcgcagaggtaagta

SEQ ID NO: 359 - DNA, 16 bp, Artificial
  Anti-sense oligonucleotide
gctattaccttaaccc

SEQ ID NO: 360 - DNA, 16 bp, Artificial
  Anti-sense oligonucleotide
gcaaattcctattccc

SEQ ID NO: 361 - DNA, 16 bp, Artificial
  Anti-sense oligonucleotide
gcacttacctattggc

SEQ ID NO: 362 - DNA, 23 bp, Artificial
  RT-PCT primer
cgtaaacggccacaagttcagcg

SEQ ID NO: 363 - DNA, 24 bp, Artificial
  RT-PCR primer
gtggtgcagatgaacttcagggtc

SEQ ID NO: 364 - DNA, 22 bp, Artificial
  RT-PCR primer
gcctaccgtggtgttcgtttcc

SEQ ID NO: 365 - DNA, 29 bp, Artificial
  RT-PCR primer
gtacatcgactgaaatccctggtaatccg

SEQ ID NO: 366 - DNA, 40 bp, Artificial
  RT-PCR primer
atctacctcccggttttaatgaatacgattttgtgccaga

SEQ ID NO: 367 - DNA, 37 bp, Artificial
  RT-PCR primer
cttcaaatctatacattaagacgactcgaaatccaca

SEQ ID NO: 368 - DNA, 29 bp, Artificial
  RT-PCR primer
ctccttcttcgccaaaagcactctgattg

SEQ ID NO: 369 - DNA, 23 bp, Artificial
  RT-PCR primer
ggacctctcacacacagttcgcc

SEQ ID NO: 370 - DNA, 23 bp, Artificial
  RT-PCR primer
ggcgaactgtgtgtgagaggtcc

SEQ ID NO: 371 - DNA, 27 bp, Artificial
  RT-PCR primer
cggtacttcgtccacaaacacaactcc

SEQ ID NO: 372 - DNA, 33 bp, Artificial
  RT-PCR primer
gctattccagaagtagtgaggaggcttttttgg

SEQ ID NO: 373 - DNA, 35 bp, Artificial
  RT-PCR primer
caccggcataaagaattgaagagagttttcactgc

SEQ ID NO: 374 - DNA, 32 bp, Artificial
  RT-PCR primer
ccgtgaaggtgcctatgatgaagcgtttagtc

SEQ ID NO: 375 - DNA, 30 bp, Artificial
  RT-PCR primer
cccctcatcaggcaggaagaagatggcggt

SEQ ID NO: 376 - DNA, 18 bp, Artificial
  S0 intron sequence
ctgggttaaggtaatagc

SEQ ID NO: 377 - DNA, 18 bp, Artificial
  Mutated intron sequence (S1)
ctgccaataggtaagtgc

SEQ ID NO: 378 - DNA, 16 bp, Artificial
  Anti-sense oligonucleotide target sequence
tgggttaaggtaatag

SEQ ID NO: 379 - DNA, 16 bp, Artificial
  Anti-sense oligonucleotide target sequence
agcgaataggtaatac

SEQ ID NO: 380 - DNA, 16 bp, Artificial
  Anti-sesnse oligonucleotide target sequence
cgagggcaggtaataa

SEQ ID NO: 381 - DNA, 16 bp, Artificial
  Anti-sense oligonucleotide target sequence
ggtgccgaggtaatat

SEQ ID NO: 382 - DNA, 16 bp, Artificial
  Anti-sense oligonucleotide target sequence
tgggttaaggtaatag

SEQ ID NO: 383 - DNA, 16 bp, Artificial
  Anti-sense oligonucleotide target sequence
ggtgactaggtaatac

SEQ ID NO: 384 - DNA, 16 bp, Artificial
  Anti-sense oligonucleotide target sequence
cgagggcaggtaatat

SEQ ID NO: 385 - DNA, 16 bp, Artificial
  Anti-sense oligonucleotide target sequence
agcgcagaggtaataa